(19) United States
(12) Patent Application Publication
(10) Pub. No.: US 2012/0094995 A1
(43) Pub. Date: Apr. 19, 2012

(54) 2-AZA-BICYCLO[2.2.1]HEPTANE COMPOUNDS AND USES THEREOF

(75) Inventors: Jeffrey Scott Albert, Montreal (CA); Donald Andisik, Wilmington, DE (US); Cristobal Alhambra, Montreal (CA); Todd Andrew Brugel, Wilmington, DE (US); Glen E Ernst, Wilmington, DE (US); William Frietze, Wilmington, DE (US); Lindsay Hinkley, Wilmington, DE (US); Jeffrey Gilbert Varnes, Boston, MA (US); Xia Wang, Wilmington, DE (US); Hui Xiong, Boston, MA (US)

(73) Assignee: ASTRAZENECA AB, Sodertalje (SE)

(21) Appl. No.: 13/146,461

(22) PCT Filed: Jan. 27, 2010
(86) PCT No.: PCT/SE2010/050072

§ 371 Date: Jan. 4, 2012

Related U.S. Application Data
(60) Provisional application No. 61/148,024, filed on Jan. 28, 2009.

(51) Int. Cl. A61K 31/407; A61P 25/18; A61P 25/22; A61P 25/00; A61K 31/506; A61P 25/04; A61K 31/4439; A61K 31/4155; A61K 31/422; A61K 31/5377; C07D 487/08; A61P 25/24

(52) U.S. Cl. 514/233.2; 548/452; 514/413; 546/276.7; 514/339; 548/468; 548/364.7; 514/406; 548/231; 514/376; 544/143; 544/335; 514/256

(57) ABSTRACT

This invention relates to 2-aza-bicyclo[2.2.1]heptane compounds (and salts thereof), the process for making such a compound and pharmaceutical compositions comprising such a compound. The invention also relates to the use of the compounds for modulating the glycine transporter 1 (GlyT1) and for the treatment of psychosis, cognitive disorders, bipolar disorders, depression disorders, anxiety disorders, post-traumatic stress disorders and pain.

### CROSS-REFERENCE TO RELATED PATENT APPLICATION

This patent claims the benefit of priority to U.S. Provisional Patent Application No. 61/148,024 (filed Jan. 28, 2009). The entire text of the above patent application is incorporated by reference into this patent.

### FIELD OF INVENTION

This invention relates to 2-aza-bicyclo[2.2.1]heptane compounds. This invention also relates to pharmaceutical compositions comprising such a compound, uses of such a compound (including, for example, treatment methods and medicament preparations), and processes for making such a compound.

### BACKGROUND

Since the discovery of the unique behavioral effects of PCP, a number of studies have been performed to evaluate the degree of similarity between the symptoms and neurocognitive deficits induced by NMDA antagonists and those observed endogenously in schizophrenia. Studies were conducted first using PCP itself, until the drug was withdrawn from the market in the late 1960s. In those studies, PCP was found to induce not only symptoms, but also neuropsychological deficits that closely resemble those of schizophrenia. More recent studies with ketamine strongly support and extend the initial observations. Such studies led to the hypothesis that the psychotic and cognitive effects experienced by both disease sufferers and people treated with NMDA antagonists resulted from reduced NMDA receptor mediated neurotransmission. This has been termed the NMDA hypofunction hypothesis for schizophrenia. According to the hypothesis, novel treatments for schizophrenia and other psychotic diseases may result from increased NMDA activation in the central nervous system. In principle, this could be achieved by treatment with direct NMDA agonists; however, such compounds are known to cause neurotoxicity. Glycine is a requisite co-agonist for NMDA receptor, and increases in its concentration may result in increased NMDA activation. The concentration of glycine is regulated by the action of the glycine transporter. Treatment with compounds that modulate the glycine transporter may increase the synaptic glycine level and thus result in NMDAr potentiation and improvement in disease symptomology.

Many people around the world continue to suffer from various psychoses and other cognitive disorders despite existing treatments. Accordingly, there is a need for new compounds and/or compositions, such as those that modulate the glycine transporter and methods of treatment of such diseases, disorders, or conditions employing such compounds or compositions.

### SUMMARY OF INVENTION

This invention relates to, inter alia, 2-aza-bicyclo[2.2.1]heptane compounds; treatment methods using the 2-aza-bicyclo[2.2.1]heptane compounds (e.g., method for treating psychosis and other cognitive disorders and as pharmacological tools); uses of the 2-aza-bicyclo[2.2.1]heptane compounds to make medicaments; compositions comprising the 22-aza-bicyclo[2.2.1]heptane compounds (e.g., pharmaceutical compositions); methods for manufacturing the 2-aza-bicyclo[2.2.1]heptane compounds; and intermediates used in such manufacturing methods.

Briefly, this invention is directed, in part, to the compound of Formula (I) or a salt thereof. Formula (I) corresponds to:

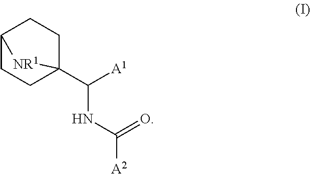

### Here:

In some embodiments, A¹ is phenyl optionally substituted with 1, 2, or 3 R⁵ groups. Alternatively, A¹ is 5- or 6-membered heteroaryl optionally substituted with 1, 2, or 3 R⁷ groups.

In some embodiments, A² is phenyl substituted with 1, 2, or 3 R² groups. Alternatively, A² is heteroaryl optionally substituted with 1, 2, or 3 R⁶ groups.

Each R is independently selected from C₁-C₆-alkyl, C₃-C₈-cycloalkyl-C₁-C₆-alkyl, and NR³R⁴.

R¹ is selected from H, C₁-C₆-alkyl, C₁-C₄-alkoxy-C₁-C₄-alkyl, amino-C₁-C₆-alkyl, cyano-C₁-C₆-alkyl, aminocarbonyl-C₁-C₆-alkyl, hydroxy-C₁-C₆-alkyl, halo-C₃-C₆-alkyl, aminocarbonyloxy-C₁-C₄-alkyl, amino-C₁-C₆-alkylcarbonyl, C₁-C₄-alkylcarbonylamino-C₁-C₄-alkyl, C₁-C₄-alkoxycarbonyl-C₁-C₄-alkyl, C₃-C₆ cycloalkyl, 3-6 membered heterocycloalkyl, 5-6 membered heteroaryl, C₃-C₈-cycloalkyl-C₁-C₄-alkyl, aryl-C₁-C₄-alkyl, heterocycloalkyl-C₁-C₄-alkyl, heteroaryl-C₁-C₄-alkyl, and C₃-C₈-alkenyl. The C₃-C₈-cycloalkyl-C₁-C₄-alkyl, aryl-C₁-C₄-alkyl, heterocycloalkyl-C₁-C₄-alkyl, and heteroaryl-C₁-C₄-alkyl, in turn, are optionally substituted with one or more substituents independently selected from halogen and C₁-C₄-alkyl. The heterocycloalkyl-C₁-C₄-alkyl also is optionally substituted with an oxo. And the amino of the amino-C₁-C₆-alkyl, aminocarbonyl-C₁-C₆-alkyl, aminocarbonyloxy-C₁-C₄-alkyl, and amino-C₁-C₆-alkylcarbonyl is optionally substituted with one or two independently selected C₁-C₄-alkyl.

Each R² is independently selected from halogen, —CN, C₂-C₆ alkenyl, C₂-C₆ alkynyl, C₃-C₆ cycloalkyl, heterocyclyl, —SOR, —SO₂R, —NH₂, —SR, C₁-C₆-alkoxy, C₁-C₆-alkyl, —CF₃, and —OCF₃. The C₁-C₆-alkyl, C₁-C₆-alkoxy, and C₃-C₆ cycloalkyl, in turn, is optionally substituted with one or more halogens. In addition, the heterocyclyl is optionally substituted with 1, 2, or 3 R⁶ groups.

Each R⁵ is independently selected from C₁-C₆-alkyl, C₃-C₈-cycloalkyl, C₁-C₆-alkoxy, —CF₃, —OCF₃, —CN, halogen, —SO₂R, —SOR, —SR, C₁-C₄-alkylcarbonylamino, hydroxy, C₁-C₄-alkoxycarbonyl, amino, aminocarbonyl, and heterocyclyl. The C₁-C₆-alkyl, C₃-C₈-cycloalkyl, and C₁-C₆-alkoxy, in turn, is optionally substituted with one or more halogens. The aminocarbonyl is optionally substituted with up to two independently selected C₁-C₄-alkyl. In addition, the heterocyclyl is optionally substituted by C₁-C₄-alkyl or halogen.

Each R⁶ is independently selected from C₁-C₆-alkyl, C₁-C₆-alkoxy, halogen, —SO₂R, —SOR, —SR, phenyl, —CF₃, —OCF₃, —CN, and heterocyclyl. The heterocyclyl, in turn, is optionally substituted by C₁-C₄-alkyl.

Each R⁷ is independently selected from C₁-C₆-alkyl, C₁-C₄-alkoxy, —CF₃, —OCF₃, —CN, —SO₂R, —SOR, —SR, phenyl, heterocyclyl, and C₁-C₄-alkoxy. The C₁-C₆-alkyl, C₃-C₈-cycloalkyl, and C₁-C₄-alkoxy, in turn, is optionally substituted with one or more halogens. In addition, the heterocyclyl is optionally substituted by C₁-C₄-alkyl or halogen.

Each R³ and R⁴ are independently selected from H and C₁-C₆-alkyl.

This invention excludes any single optical isomer, racemic mixture, or other mixture of optical isomers corresponding to a structure selected from the following (or a salt thereof):

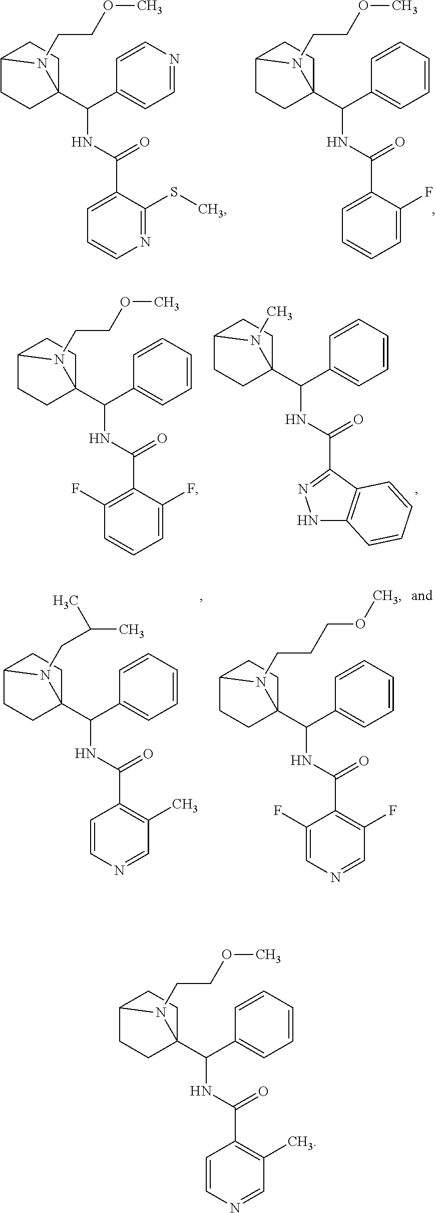

This invention also is directed, in part, to a pharmaceutical composition. The composition comprises a compound of Formula (I) or a pharmaceutically acceptable salt thereof. The composition also comprises a pharmaceutically acceptable carrier or diluent.

This invention also is directed, in part, to a compound of Formula (I) or a pharmaceutically acceptable salt thereof for use in treating a condition (typically a disorder).

This invention also is directed, in part, to a method of using a compound of Formula (I) or a pharmaceutically acceptable salt thereof to treat a condition.

This invention also is directed, in part, to a method of treating a condition in a patient in need of such treatment. The method comprises administering a compound of Formula (I) or a pharmaceutically acceptable salt thereof to the patient.

This invention also is directed, in part, to a use of a compound of Formula (I) or a pharmaceutically acceptable salt thereof in the manufacture of a medicament (e.g., a pharmaceutical composition) for treating a condition.

Further benefits of Applicants' invention will be apparent to one skilled in the art from reading this specification.

### DETAILED DESCRIPTION

This description of illustrative embodiments is intended only to acquaint others skilled in the art with Applicants' invention, its principles, and its practical application so that others skilled in the art may readily adapt and apply the invention in its numerous forms, as they may be best suited to the requirements of a particular use. This description and its specific examples, while indicating embodiments of this invention, are intended for purposes of illustration only. This invention, therefore, is not limited to the illustrative embodiments described in this specification, and may be variously modified. In addition, it is to be appreciated that various features of the invention that are, for clarity reasons, described in the context of separate embodiments, also may be combined to form a single embodiment. Conversely, various features of the invention that are, for brevity reasons, described in the context of a single embodiment, also may be combined to form sub-combinations thereof

As noted above, this invention is directed, in part, to the compound of Formula (I) or a salt thereof. Formula (I) corresponds to:

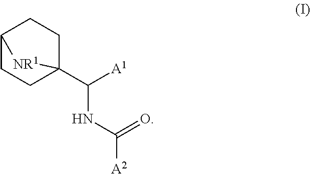

The substituents of Formula (I) are defined as follows:

In some embodiments, A¹ is phenyl (i.e., unsubstituted phenyl). In these embodiments, the compound corresponds to Formula (II):

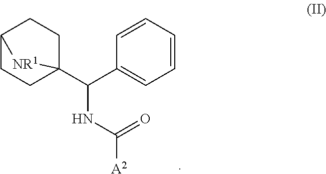

In some embodiments, A¹ is phenyl substituted with 1, 2, or 3 R⁵ groups. In some such embodiments, A¹ is phenyl substituted with 1 R⁵ group. In other embodiments, A¹ is phenyl substituted with 2 R⁵ groups. And in other embodiments, A¹ is phenyl substituted with 3 R⁵ groups.

In some embodiments, A¹ is a 5- or 6-membered heteroaryl (i.e., unsubstituted 5- or 6-membered heteroaryl). In some embodiments, the heteroaryl is 5-membered. In other embodiments, the heteroaryl is 6-membered. In some such embodiments, for example, the heteroaryl is pyridinyl. In other embodiments, the heteroaryl is pyrimidinyl.

In some embodiments, A¹ is a 5- or 6-membered heteroaryl substituted with 1, 2, or 3 R⁷ groups. In some such embodiments, A¹ is 5- or 6-membered heteroaryl substituted with 1 R⁷ group. In other embodiments, A¹ is 5- or 6-membered heteroaryl substituted with 2 R⁷ groups. And in other embodiments, A¹ is 5- or 6-membered heteroaryl substituted with 3 R⁷ groups. In some embodiments, the heteroaryl that is substituted is 5-membered. In some such embodiments, for example, the heteroaryl that is substituted is furanyl. In other embodiments, the heteroaryl that is substituted is pyrazolyl. In some embodiments, the heteroaryl that is substituted is 6-membered. In some such embodiments, for example, the heteroaryl that is substituted is pyridinyl.

In some embodiments, A² is phenyl substituted with 1, 2, or 3 R² groups. In some such embodiments, A² is a phenyl substituted with 1 R² group. In other embodiments, A² is a phenyl substituted with 2 R² groups. And in other embodiments, A² is a phenyl substituted with 3 R² groups.

In some embodiments, A² is a heteroaryl (i.e., unsubstituted heteroaryl). In some embodiments, the heteroaryl is 5-membered. In some embodiments, the heteroaryl is 6-membered. In some embodiments, the heteroaryl is 9-membered. In some such embodiments, for example, A² is indazolyl.

In some embodiments, A² is heteroaryl substituted with 1, 2, or 3 R⁶ groups. In some such embodiments, A² is a heteroaryl substituted with 1 R⁶ group. In other embodiments, A² is a heteroaryl substituted with 2 R⁶ groups. And in other embodiments, A² is a heteroaryl substituted with 3 R⁶ groups. In some embodiments, the heteroaryl that is substituted is 5-membered. In some embodiments, the heteroaryl that is substituted is 6-membered. In some such embodiments, for example, the heteroaryl is pyridinyl. In some such embodiments, for example, the heteroaryl is pyrimidinyl. In some embodiments, the heteroaryl that is substituted is 9-membered.

In the above embodiments, each R is independently selected from C₁-C₆-alkyl, C₃-C₈-cycloalkyl-C₁-C₆-alkyl, and NR³R⁴.

In some such embodiments, R is C₁-C₆-alkyl. In some such embodiments, R is methyl. In other embodiments, R is ethyl. And, in other embodiments, R is propyl.

In some such embodiments, R is C₃-C₈-cycloalkyl-C₁-C₆-alkyl.

In some such embodiments, R is NR³R⁴.

R¹ is selected from H, C₁-C₆-alkyl, C₁-C₄-alkoxy-C₁-C₄-alkyl, amino-C₁-C₆-alkyl, cyano-C₁-C₆-alkyl, aminocarbonyl-C₁-C₆-alkyl, hydroxy-C₁-C₆-alkyl, halo-C₃-C₆-alkyl, aminocarbonyloxy-C₁-C₄-alkyl, amino-C₁-C₆-alkylcarbonyl, C₁-C₄-alkylcarbonylamino-C₁-C₄-alkyl, C₁-C₄-alkoxycarbonyl-C₁-C₄-alkyl, C₃-C₆ cycloalkyl, 3-6 membered heterocycloalkyl, 5-6 membered heteroaryl, C₃-C₈-cycloalkyl-C₁-C₄-alkyl, aryl-C₁-C₄-alkyl, heterocycloalkyl-C₁-C₄-alkyl, heteroaryl-C₁-C₄-alkyl, and C₃-C₈-alkenyl. The C₃-C₈-cycloalkyl-C₁-C₄-alkyl, aryl-C₁-C₄-alkyl, heterocycloalkyl-C₁-C₄-alkyl, and heteroaryl-C₁-C₄-alkyl, in turn, are optionally substituted with one or more substituents independently selected from halogen and C₁-C₄-alkyl. In addition, the heterocycloalkyl-C₁-C₄-alkyl is optionally substituted with an oxo. And the amino of the amino-C₁-C₆-alkyl, aminocarbonyl-C₁-C₆-alkyl, aminocarbonyloxy-C₁-C₄-alkyl, and amino-C₁-C₆-alkylcarbonyl is optionally substituted with one or two independently selected C₁-C₄-alkyl.

In some embodiments, R¹ is C₁-C₄-alkoxy-C₁-C₄-alkyl. In some such embodiments, for example, R¹ is methoxyethyl. In other embodiments, R¹ is methoxypropyl.

In some embodiments, R¹ is hydroxy-C₁-C₆-alkyl. In some such embodiments, for example, R¹ is 2-hydroxyethyl.

In some embodiments, R¹ is cyano-C₁-C₆-alkyl. In some such embodiments, for example, R¹ is cyanomethyl.

In some embodiments, R¹ is amino-C₁-C₆-alkyl. In some such embodiments, for example, R¹ is 2-aminoethyl. In other embodiments, for example, R¹ is 2-aminopropyl

In some embodiments, R¹ is C₁-C₄-alkylcarbonylamino-C₁-C₄-alkyl. In some such embodiments, for example, R¹ is methylcarbonylaminoethyl.

In some embodiments, R¹ is aminocarbonyl-C₁-C₆-alkyl, wherein the amino is optionally substituted with one or two independently selected C₁-C₄-alkyl. In some such embodiments, for example, R¹ is dimethylaminocarbonylmethyl. In other embodiments, for example, R¹ is aminocarbonylmethyl.

In some embodiments, R¹ is amino-C₁-C₆-alkylcarbonyl, wherein the amino is optionally substituted with one or two independently selected C₁-C₄-alkyl. In some such embodiments, for example, R¹ is dimethylaminomethylcarbonyl. In other embodiments, R¹ is aminomethylcarbonyl.

In some embodiments, R¹ is aminocarbonyloxy-C₁-C₄-alkyl, wherein the amino is optionally substituted with one or two independently selected C₁-C₄-alkyl. In some such embodiments, for example, R¹ is dimethylaminocarbonyloxyethyl.

In some embodiments, R¹ is C₁-C₄-alkoxycarbonyl-C₁-C₄-alkyl. In some such embodiments, for example, R¹ is ethoxycarbonylmethyl.

In some embodiments, R¹ is selected from H, C₁-C₆-alkyl, C₃-C₆-cycloalkyl, 3-6 membered heterocycloalkyl, C₃-C₈-cycloalkyl-C₁-C₄-alkyl, aryl-C₁-C₄-alkyl, heterocycloalkyl-C₁-C₄-alkyl, heteroaryl-C₁-C₄-alkyl, and C₃-C₈-alkenyl. The C₃-C₈-cycloalkyl-C₁-C₄-alkyl, aryl-C₁-C₄-alkyl, heterocycloalkyl-C₁-C₄-alkyl, heteroaryl-C₁-C₄-alkyl, in turn, are optionally substituted with one or more independently selected halogen.

In some embodiments, R¹ is C₃-C₆ cycloalkyl. In some such embodiments, R¹ is cyclopropyl. In other embodiments, R¹ is cyclobutyl.

In some embodiments, R¹ is C₃-C₈-cycloalkyl-C₁-C₄-alkyl. In some embodiments, for example, R¹ is cyclopropylmethyl.

In some embodiments, R¹ is C₃-C₈-cycloalkyl-C₁-C₄-alkyl substituted with one or more independently selected halogen.

In some embodiments, R¹ is aryl-C₁-C₄-alkyl. In some embodiments, for example, R¹ is phenylmethyl.

In some embodiments, R¹ is heterocyclyl-C₁-C₄-alkyl. In some such embodiments, for example, R¹ is pyrrolidinylmethyl. In other embodiments, R¹ is pyrrolidinylethyl. In other embodiments, R¹ is tetrahydrofuranylmethyl. In other embodiments, R¹ is morpholinylethyl.

In some embodiments, R¹ is heterocycloalkyl-C₁-C₄-alkyl is optionally substituted with an oxo. In some embodiments, for example, R¹ is 2-oxo-oxazolidinyl.

In some embodiments, R¹ is heteroaryl-C₁-C₄-alkyl. In some such embodiments, for example, R¹ is pyridinylmethyl.

In some embodiments, R¹ is heteroaryl-C₁-C₄-alkyl substituted with one or more substituents independently selected from halogen and C₁-C₄-alkyl. In some such embodiments, for example, R¹ is methylpyrazolylmethyl.

In some embodiments, R¹ is selected from aryl-C₁-C₄-alkyl, heterocyclyl-C₁-C₄-alkyl, and heteroaryl-C₁-C₄-alkyl. The aryl-C₁-C₄-alkyl, heterocyclyl-C₁-C₄-alkyl, and heteroaryl-C₁-C₄-alkyl, in turn, are substituted with one or more independently selected halogen.

In some embodiments, R¹ is selected from H, C₁-C₆-alkyl, C₃-C₆-cycloalkyl, 3-6 membered heterocycloalkyl, C₃-C₈-cycloalkyl-C₁-C₄-alkyl, aryl-C₁-C₄-alkyl, heterocycloalkyl-C₁-C₄-alkyl, heteroaryl-C₁-C₄-alkyl, and C₃-C₈-alkenyl.

In some embodiments, R¹ is hydrogen.

In some embodiments, R¹ is C₁-C₆-alkyl. In some such embodiments, for example, R¹ is methyl. In other embodiments, R¹ is ethyl. In other embodiments, R¹ is propyl. In still other embodiments, R¹ is butyl. And in still yet other embodiments, R¹ is pentyl.

In some embodiments, R¹ is halo-C₃-C₆-alkyl. In some such embodiments, for example, R¹ is 3,3,3-trifluoropropyl.

In some embodiments, R¹ is C₃-C₈-alkenyl.

In some embodiments, R¹ is heterocycloalkyl. In some such embodiments, for example, the heterocycloalkyl is a 3- to 6-membered ring.

In some embodiments, R¹ is heteroaryl. In some such embodiments, for example, the heteroaryl is a 5-membered ring. In other embodiments, the heteroaryl is a 6-membered ring.

Each R² is independently selected from halogen, —CN, C₂-C₆ alkenyl, C₂-C₆ alkynyl, C₃-C₆ cycloalkyl, heterocyclyl, —SOR, —SO₂R, —NH₂, —SR, C₁-C₆-alkoxy, C₁-C₆-alkyl, —CF₃, and —OCF₃. The C₁-C₆-alkyl, C₁-C₆-alkoxy, and C₃-C₆ cycloalkyl, in turn, are optionally substituted with one or more halogens. And the heterocyclyl is optionally substituted with 1, 2, or 3 R⁶ groups.

In some embodiments, at least one R² group is C₁-C₆-alkyl. In some such embodiments, for example, at least one R² group is methyl. In other embodiments, at least one R² group is ethyl.

In some embodiments, at least two R² groups are independently selected C₁-C₆-alkyl. In some such embodiments, for example, at least two R² groups are methyl.

In some embodiments, at least one R² group is C₁-C₆-alkyl optionally substituted with one or more independently selected halogen. In some such embodiments, for example, at least one R² group is trifluoromethyl.

In some embodiments, at least one R² group is C₁-C₆-alkoxy. In some such embodiments, for example, at least one R² group is methoxy.

In some embodiments, at least two R² groups are independently selected C₁-C₆-alkoxy. In some such embodiments, for example, at least two R² groups are methoxy.

In some embodiments, at least one R² group is halogen. In some such embodiments, for example, at least one R² group is fluoro. In other embodiments, for example, at least one R² group is chloro. In other embodiments, for example, at least one R² group is bromo.

In some embodiments, at least two R² groups are independently selected halogen. In some such embodiments, for example, at least two R² groups are chloro.

In other embodiments, at least two R² groups are present, and the R² groups are not all identical. For example, in some embodiments, one R² group is methyl and one R² group is trifluoromethyl. In other embodiments, one R² group is chloro and one R² group is methyl. In other embodiments, one R² group is chloro and one R² group is fluoro. In other embodiments, one R² group is chloro and one R² group is trifluoromethyl. In other embodiments, one R² group is fluoro and one R² group is trifluoromethyl. In other embodiments, one R² group is chloro and one R² group is methyl. In other embodiments, one R² group is fluoro and one R² group is methyl. In other embodiments, one R² group is fluoro and one R² group is amino. And in other embodiments, one R² group is fluoro and two R² groups are methyl.

Each R³ and R⁴ are independently selected from H and C₁-C₆-alkyl. In some embodiments, each of R³ and R⁴ are H. In other embodiments, each R³ and R⁴ are independently selected C₁-C₆-alkyl. And, in other embodiments, R³ is H, and R⁴ is C₁-C₆-alkyl.

Each R⁵ is independently selected from C₁-C₆-alkyl, C₃-C₈-cycloalkyl, C₁-C₆-alkoxy, —CF₃, —OCF₃, —CN, halogen, —SO₂R, —SOR, —SR, C₁-C₄-alkylcarbonylamino, hydroxy, C₁-C₄-alkoxycarbonyl, amino, aminocarbonyl, and heterocyclyl. The C₁-C₆-alkyl, C₃-C₈-cycloalkyl, and C₁-C₆-alkoxy, in turn, are optionally substituted with one or more halogens. The aminocarbonyl is optionally substituted with up to two independently selected C₁-C₄-alkyl. And the heterocyclyl is optionally substituted by C₁-C₄-alkyl or halogen.

In some embodiments, each R⁵ is independently selected from C₁-C₆-alkyl, C₃-C₈-cycloalkyl, C₁-C₆-alkoxy, —CF₃, —OCF₃, —CN, halogen, —SO₂R, —SOR, —SR, and heterocyclyl. The C₁-C₆-alkyl, C₃-C₈-cycloalkyl, and C₁-C₆-alkoxy, in turn, are optionally substituted with one or more halogens. And the heterocyclyl is optionally substituted by C₁-C₄-alkyl or halogen.

In some embodiments, at least one R⁵ group is halogen. In some such embodiments, for example, at least one R⁵ is bromo. In other embodiments, at least one R⁵ is fluoro. In other embodiments, at least one R⁵ is chloro.

In some embodiments, at least one R⁵ group is cyano (i.e., —CN).

In some embodiments, at least one R⁵ group is hydroxy (i.e., —OH).

In some embodiments, at least one R⁵ group is amino (i.e., —NH₂).

In some embodiments, at least one R⁵ group is C₁-C₆-alkyl. In some such embodiments, for example, at least one R⁵ group is methyl. In other embodiments, at least one R⁵ group is butyl.

In some embodiments, at least one R⁵ group is C₁-C₆-alkoxy. In some such embodiments, for example, at least one R⁵ group is propoxy.

In some embodiments, at least one R⁵ group is heterocyclyl. In some such embodiments, for example, at least one R⁵ group is heterocycloalkyl, such as, for example, morpholinyl.

In some embodiments, at least one R⁵ group is C₁-C₄-alkoxycarbonyl. In some such embodiments, for example, at least one R⁵ group is propoxycarbonyl.

In some embodiments, at least one R⁵ group is aminocarbonyl optionally substituted with up to two independently selected C₁-C₄-alkyl. In some such embodiments, for example, at least one R⁵ group is di-(methyl)aminocarbonyl.

In some embodiments, at least one R⁵ group is C₁-C₄-alkylcarbonylamino. In some such embodiments, for example, at least one R⁵ group is methylcarbonylamino.

Each R⁶ is independently selected from C₁-C₆-alkyl, C₁-C₆-alkoxy, halogen, —SO₂R, —SOR, —SR, phenyl, —CF₃, —OCF₃, —CN, and heterocyclyl. The heterocyclyl, in turn, is optionally substituted by C₁-C₄-alkyl.

In some embodiments, at least one R⁶ group is C₁-C₆-alkyl. In some such embodiments, for example, at least one R⁶ group is methyl.

In some embodiments, at least two R⁶ groups are independently selected C₁-C₆-alkyl. In some such embodiments, for example, at least two R⁶ groups are methyl.

In some embodiments, at least one R⁶ group is —CF₃.

In some embodiments, at least one R⁶ group is halogen. In some such embodiments, for example, at least one R⁶ group is chloro. In other embodiments, at least one R⁶ group is bromo.

In some embodiments, at least two R⁶ groups are independently selected halogen. In some such embodiments, for example, at least two R⁶ groups are chloro. In some such embodiments, for example, at least two R⁶ groups are fluoro.

In some embodiments, at least one R⁶ is —SR. In some such embodiments, for example, at least one R⁶ is methylsulfanyl (or “methylthio” or —SCH₃).

In other embodiments, at least two R⁶ groups are present, and the R⁶ groups are not all identical. For example, in some embodiments, one R⁶ group is fluoro and one R⁶ group is —CF₃.

Each R⁷ is independently selected from C₁-C₆-alkyl, C₁-C₄-alkoxy, —CF₃, —OCF₃, —CN, —SO₂R, —SOR, —SR, phenyl, heterocyclyl, and C₁-C₄-alkoxy. The C₁-C₆-alkyl, C₃-C₈-cycloalkyl, and C₁-C₄-alkoxy, in turn, are optionally substituted with one or more halogens. And the heterocyclyl is optionally substituted by C₁-C₄-alkyl or halogen;

In some embodiments, at least one R⁷ group is C₁-C₆-alkyl. In some such embodiments, at least one R⁷ group is methyl.

In some embodiments, A¹ is phenyl; and A² is phenyl substituted with 1, 2, or 3 R² groups.

In some embodiments, A¹ is phenyl (i.e., the compound corresponds in structure to Formula (II)), and A² is heteroaryl.

In some embodiments, A¹ is phenyl substituted with 1, 2, or 3 R⁵ groups; and A² is phenyl substituted with 1, 2, or 3 R² groups.

In some embodiments, A¹ is phenyl substituted with 1, 2, or 3 R⁵ groups; and A² is a heteroaryl.

In some embodiments, A¹ is phenyl substituted with 1, 2, or 3 R⁵ groups; and A² is a heteroaryl substituted with 1, 2, or 3 R⁶ groups.

In some embodiments, A¹ is a 5- or 6-membered heteroaryl; and A² is phenyl substituted with 1, 2, or 3 R² groups.

In some embodiments, A¹ is a 5- or 6-membered heteroaryl, and A² is a heteroaryl.

In some embodiments, A¹ is a 5- or 6-membered heteroaryl; and A² is a heteroaryl substituted with 1, 2, or 3 R⁶ groups.

In some embodiments, A¹ is a 5- or 6-membered heteroaryl substituted with 1, 2, or 3 R⁷ groups; and A² is phenyl substituted with 1, 2, or 3 R² groups.

In some embodiments, A¹ is a 5- or 6-membered heteroaryl substituted with 1, 2, or 3 R⁷ groups; and A² is a heteroaryl.

In some embodiments, A¹ is a 5- or 6-membered heteroaryl substituted with 1, 2, or 3 R⁷ groups; and A² is a heteroaryl substituted with 1, 2, or 3 R⁶ groups.

In some embodiments, the compound or salt is a compound or salt described in Table 1 below.

In some embodiments, the compound or salt is a compound corresponding in to the non-salt structure shown in Table 1 below or a pharmaceutically acceptable salt thereof

In some embodiments, the compound or salt is a compound shown in Table 2 below or a pharmaceutically acceptable salt thereof.

In some embodiments, the compound or salt is a compound shown in Table 3 below or a pharmaceutically acceptable salt thereof.

In some embodiments, the compound or salt is a single optical isomer, a racemic mixture, or any other mixture of optical isomers corresponding to a structure below or a pharmaceutically acceptable salt of such an isomer, racemic mixture, or other mixture of optical isomers:

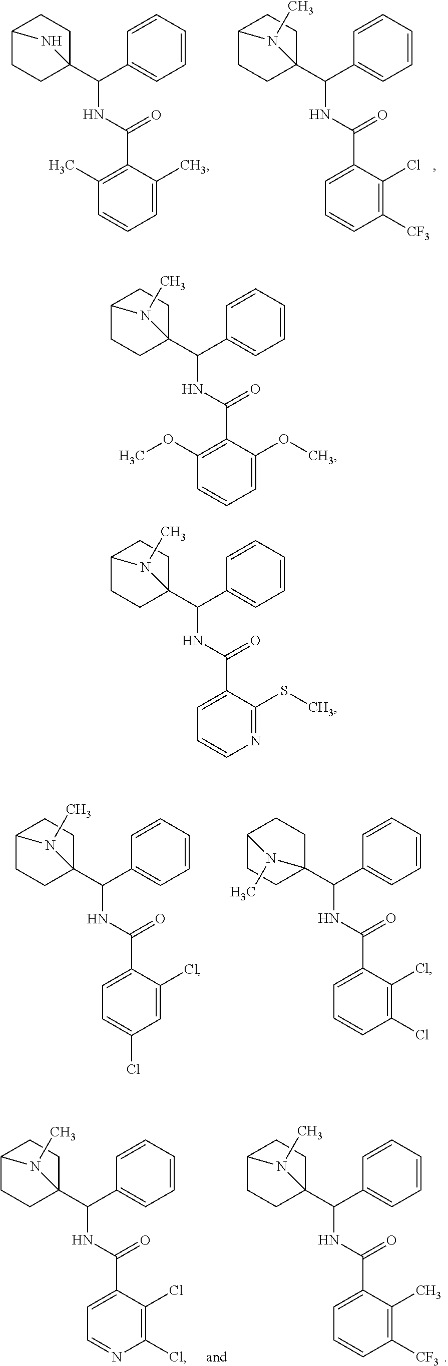

In some embodiments, the compound or salt is a single optical isomer, a racemic mixture, or any other mixture of optical isomers corresponding to a structure below or a pharmaceutically acceptable salt of such an isomer, racemic mixture, or other mixture of optical isomers:

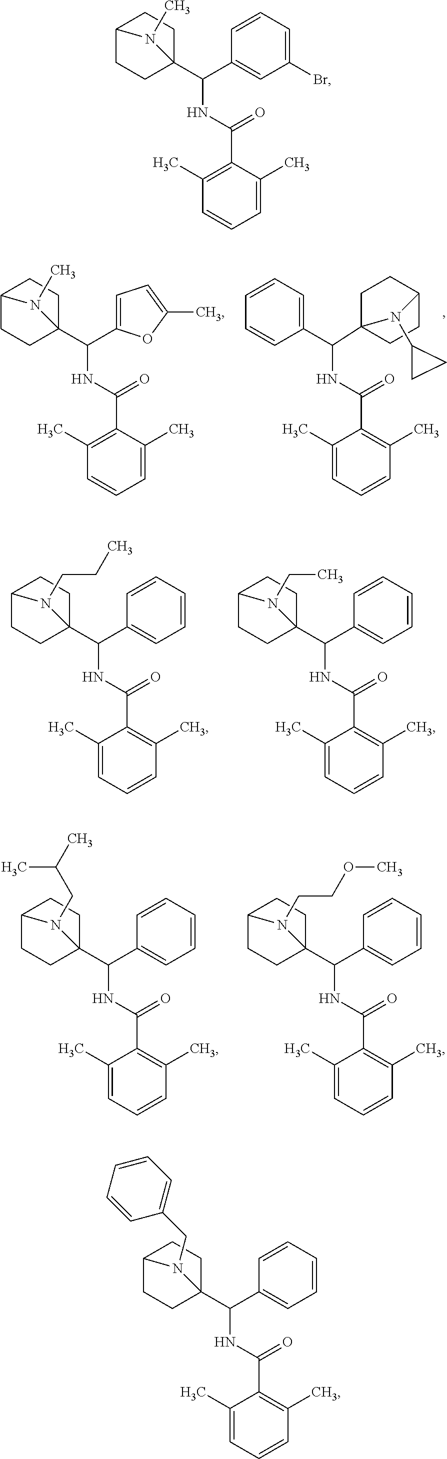

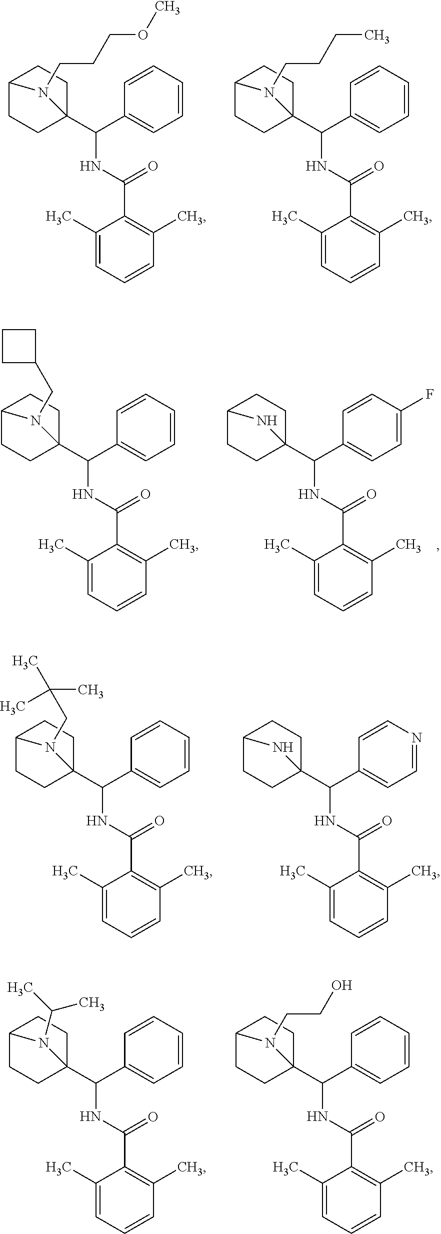

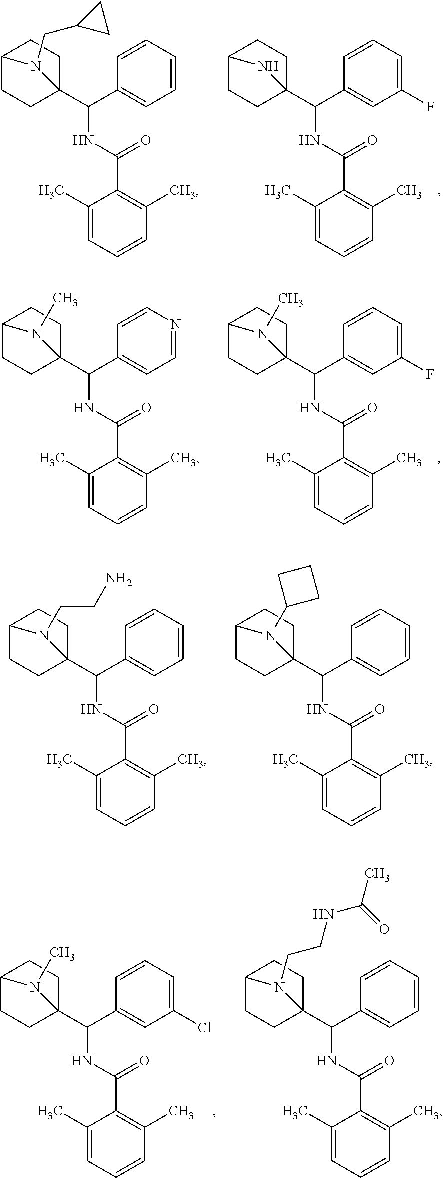

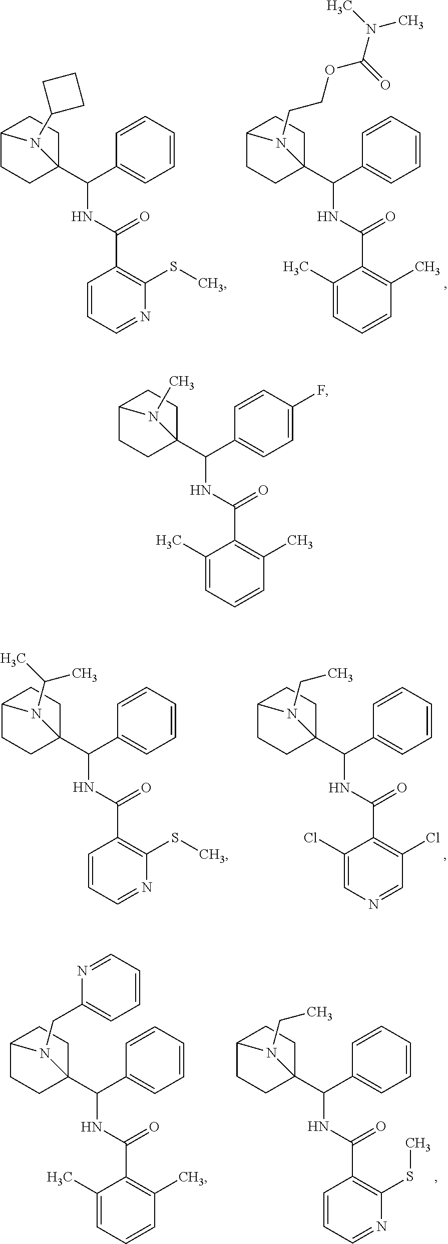

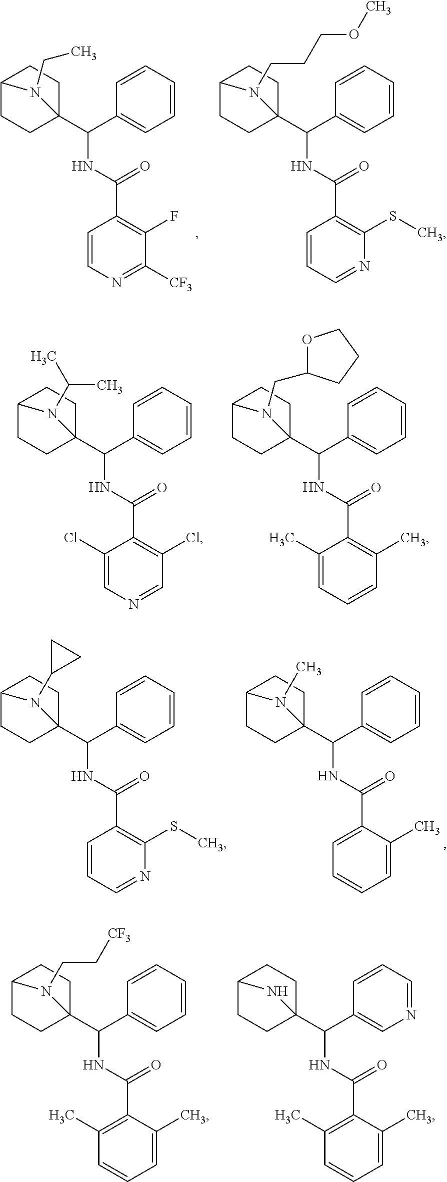

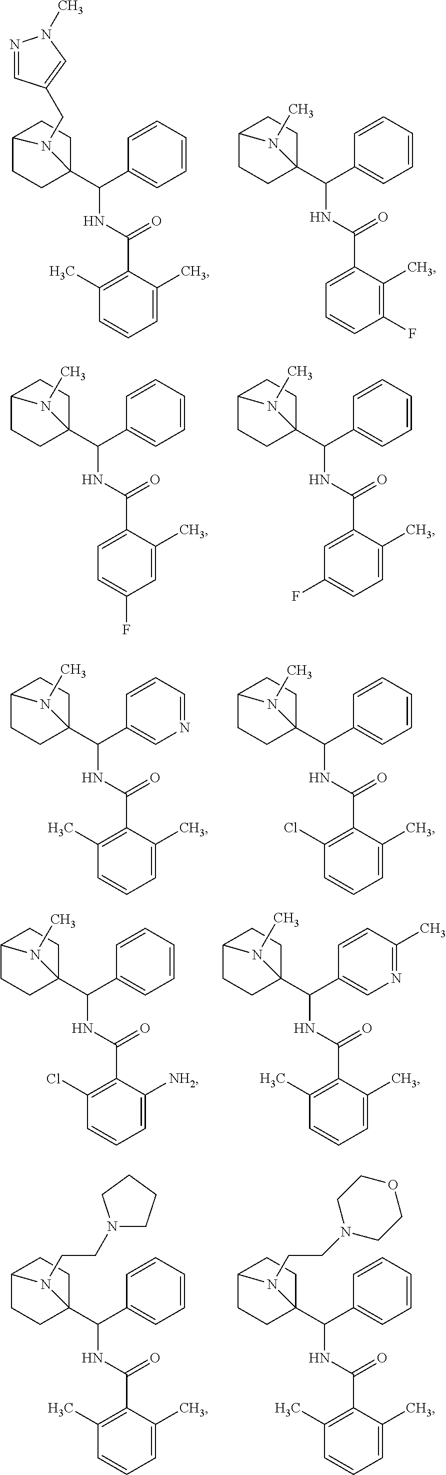

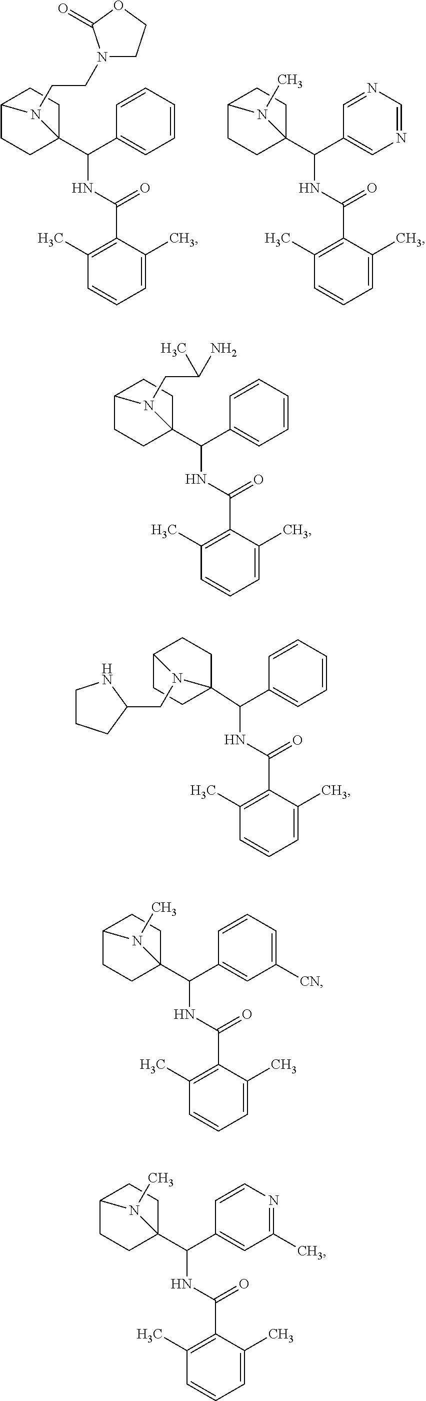

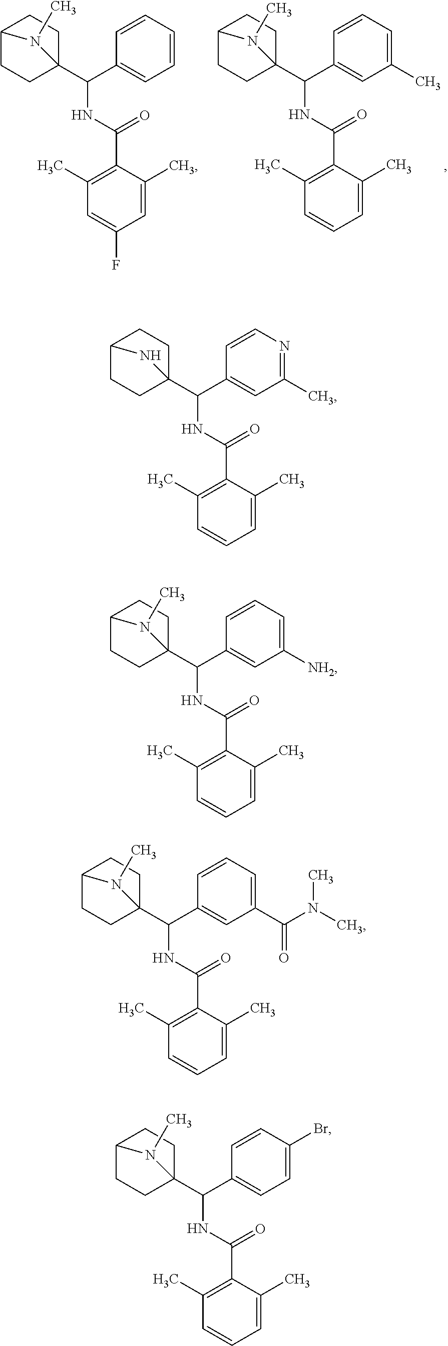

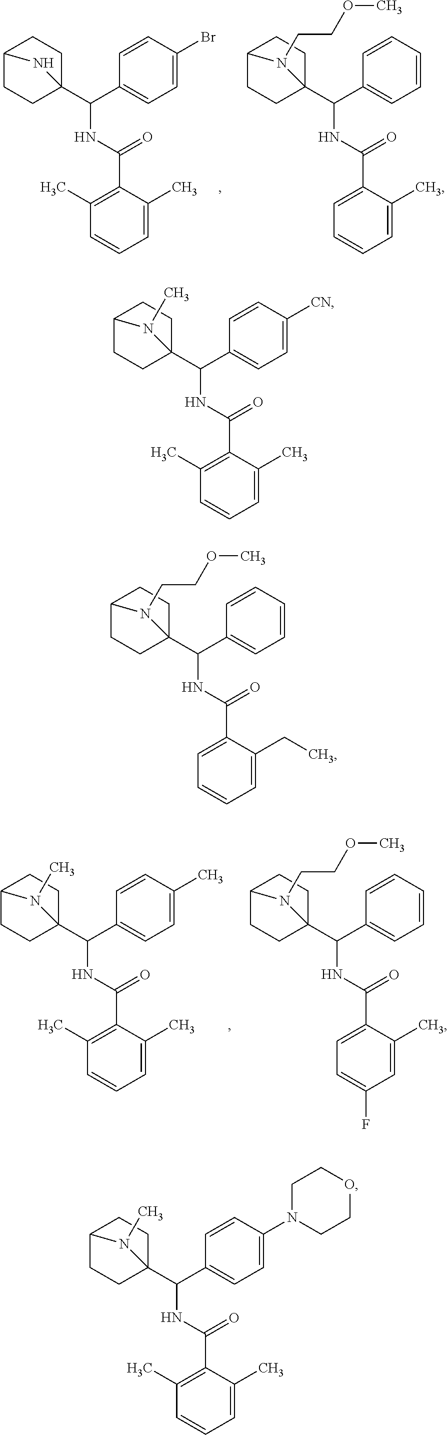

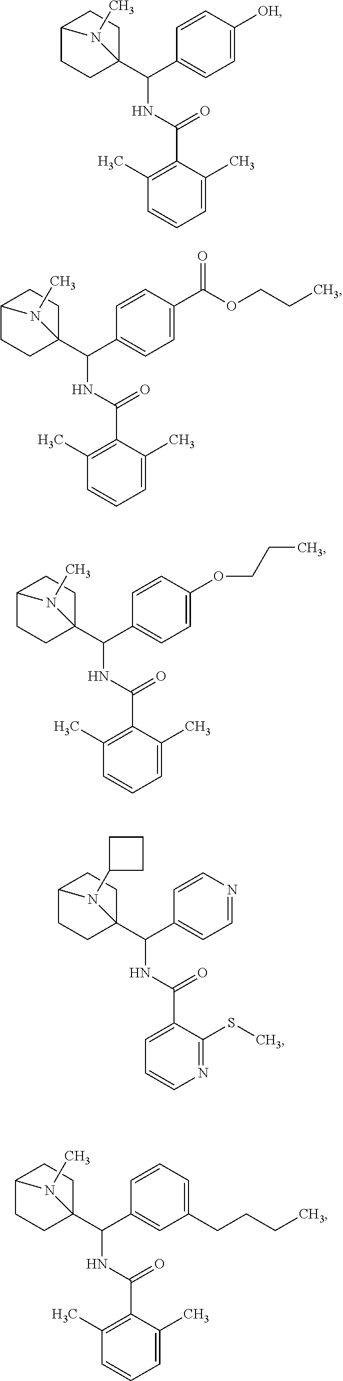

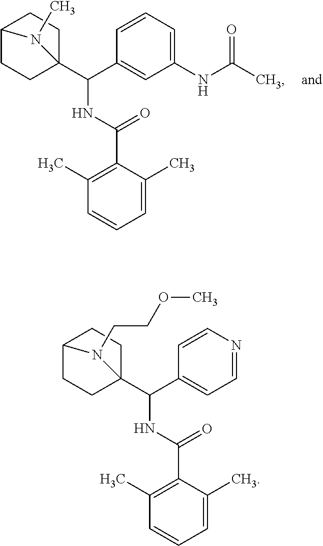

In some embodiments, the compound or salt is a single optical isomer, a racemic mixture, or any other mixture of optical isomers corresponding to a structure below or a pharmaceutically acceptable salt of such an isomer, racemic mixture, or other mixture of optical isomers:

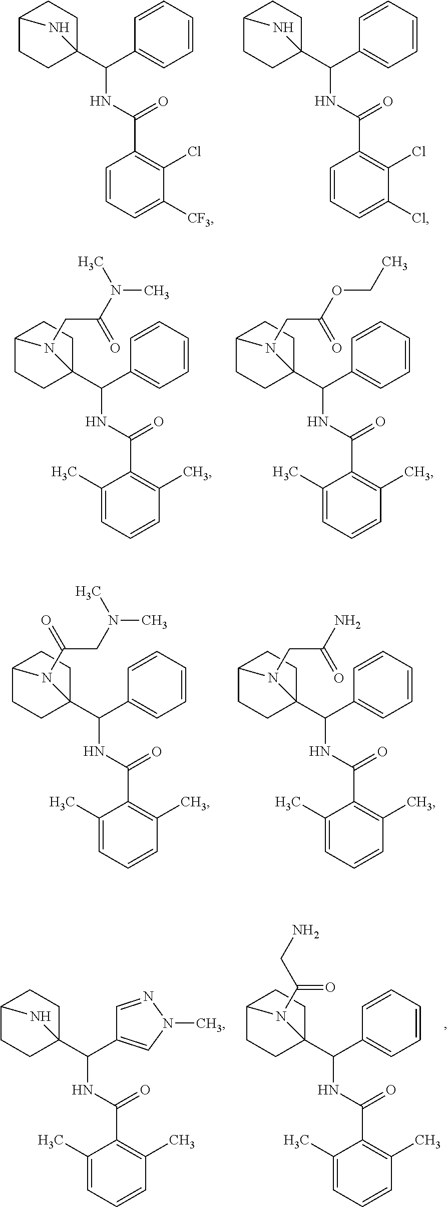

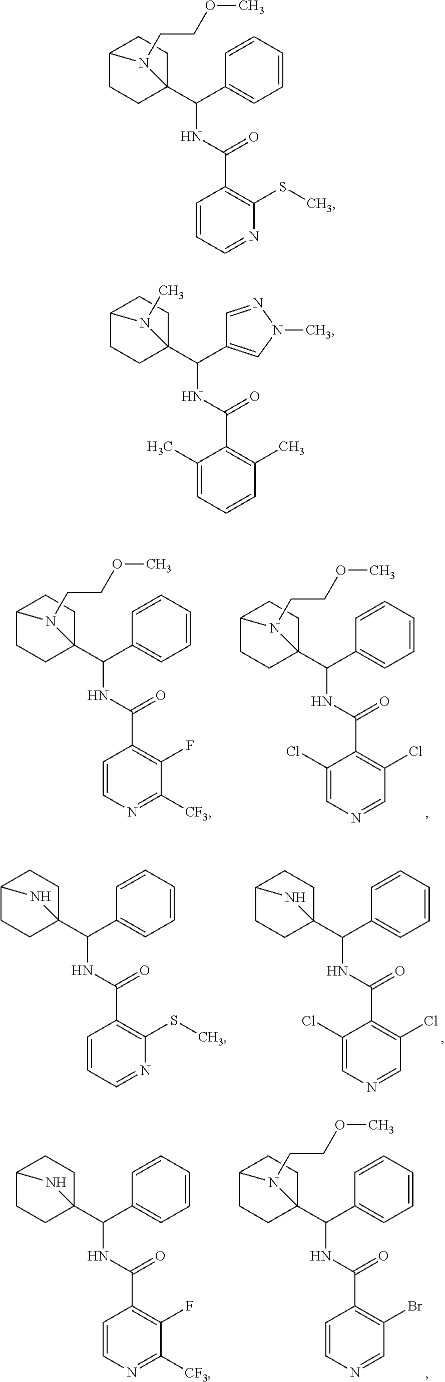

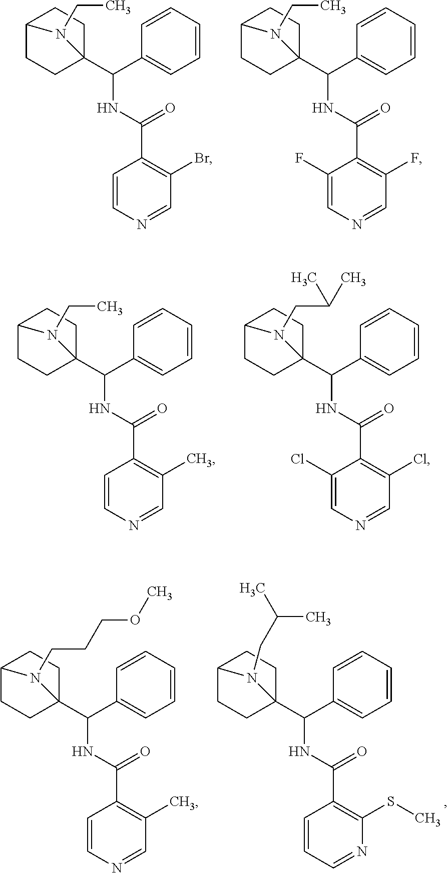

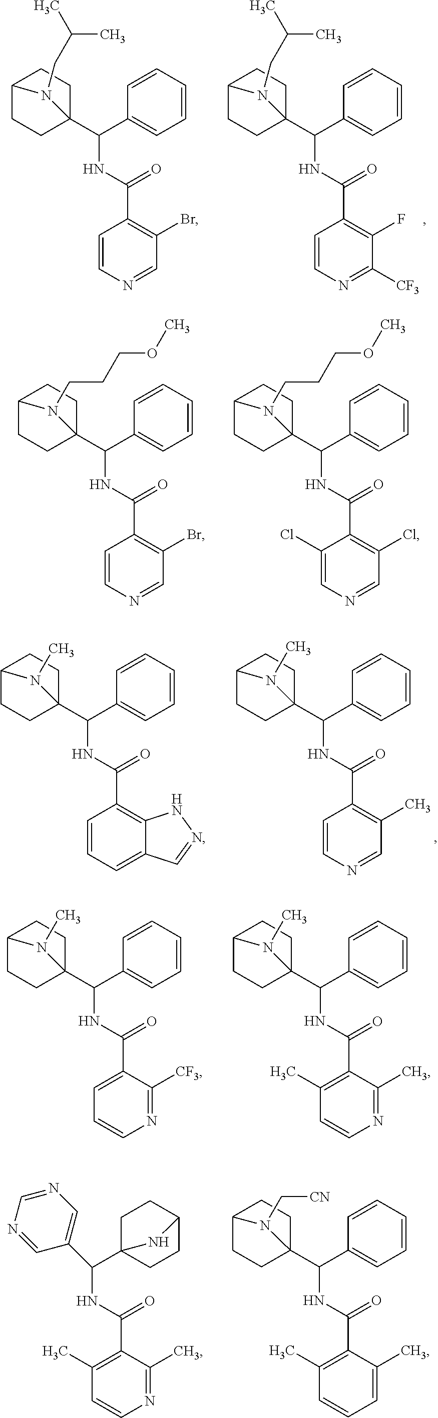

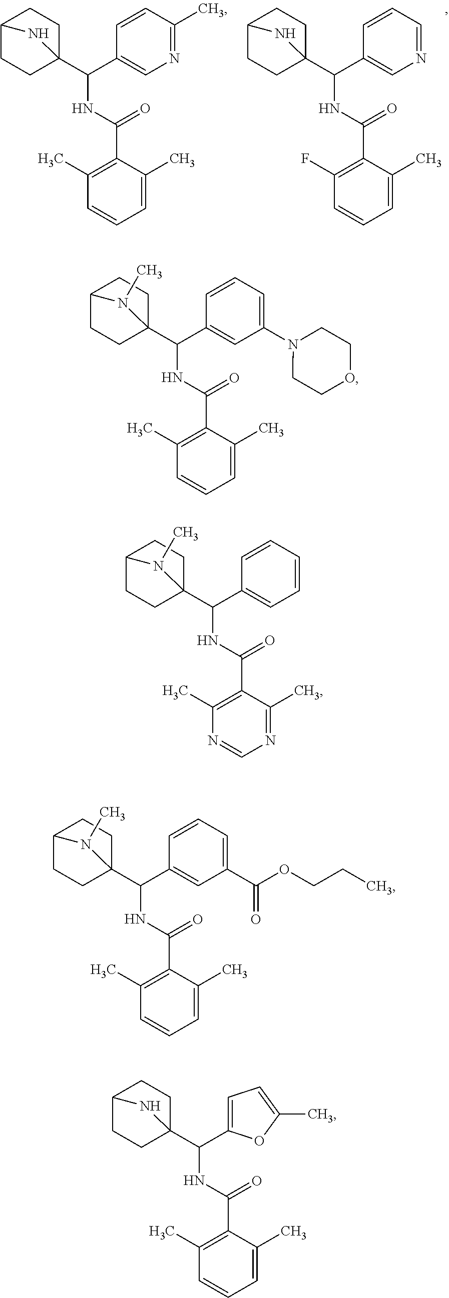

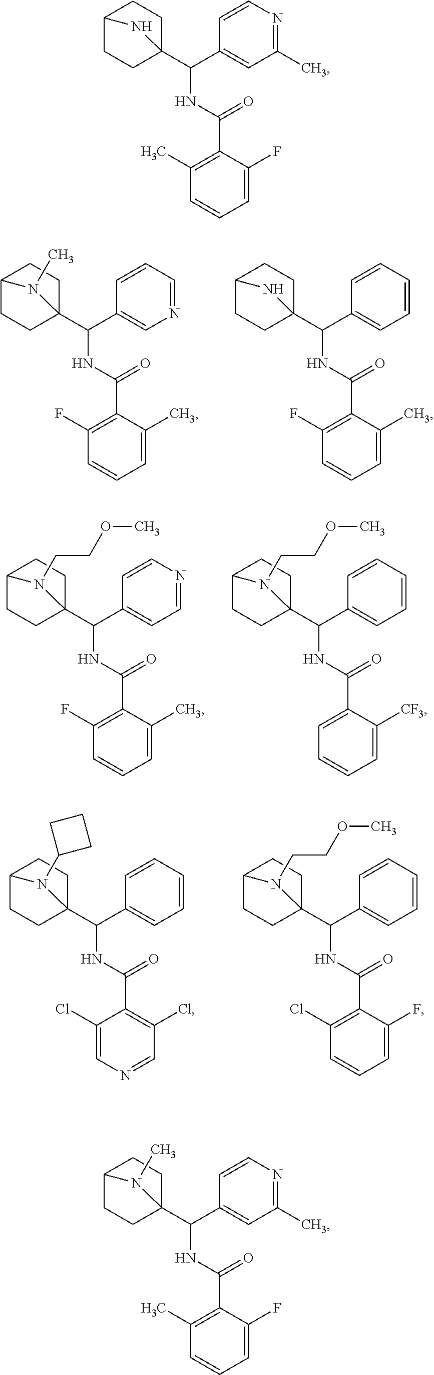

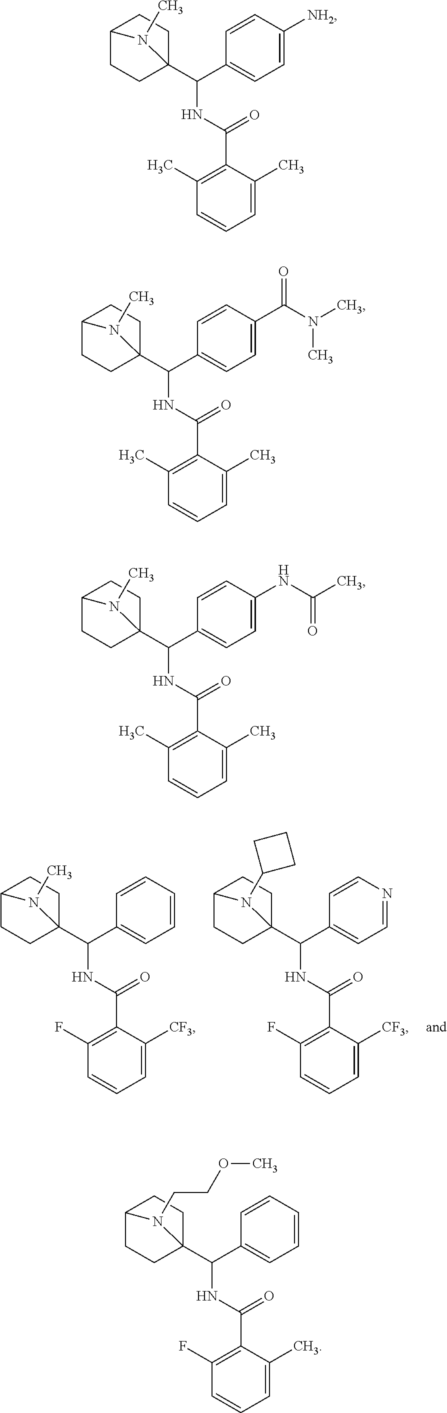

This invention excludes any single optical isomer, racemic mixture, or other mixture of optical isomers corresponding to a structure selected from the following (or a salt thereof):

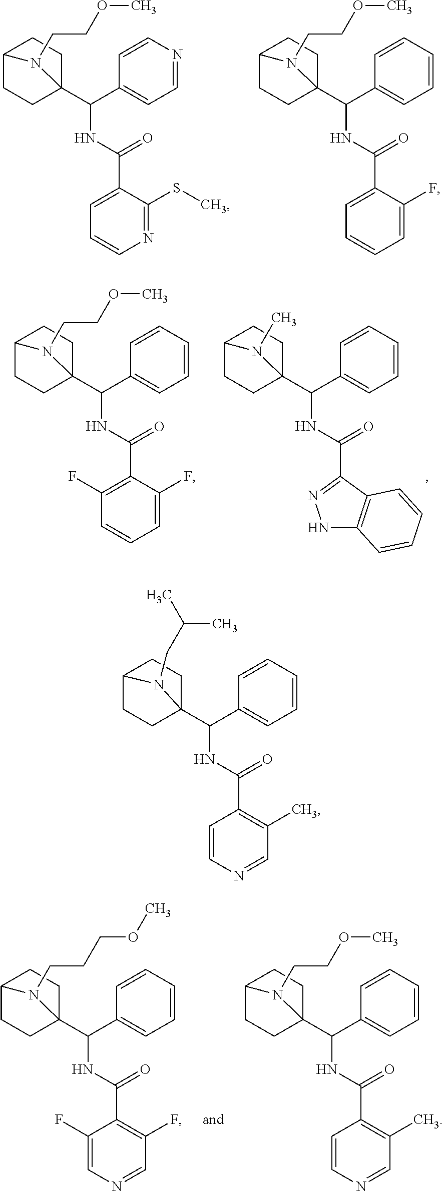

In some embodiments, the compound comprises a single optical isomer, racemic mixture, or other mixture of optical isomers corresponding to the following structure:

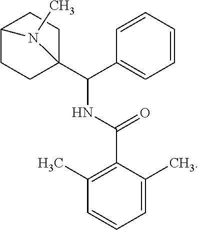

In some embodiments, the compound comprises a single optical isomer, racemic mixture, or other mixture of optical isomers corresponding to the following structure:

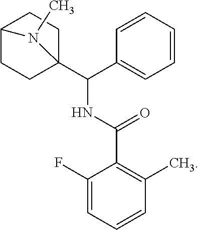

All the compounds of this invention include at least one chiral carbon, i.e., the carbon linking the 2-aza-bicyclo[2.2.1]heptane group with A¹ and the amino:

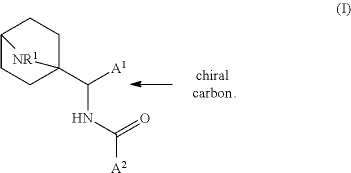

Formula (I) is intended to encompass any single chiral isomer corresponding to Formula (I), as well as any mixture of chiral isomers (e.g., the racemate) corresponding to Formula (I). Thus, Formula (I) encompasses a single chiral isomer corresponding to Formula (IA):

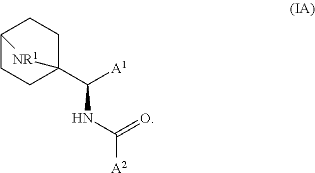

Formula (I) also encompasses a single chiral isomer corresponding to Formula (IB):

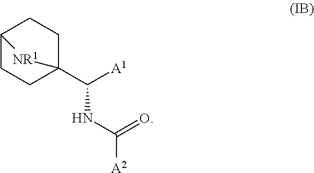

Formula (I) also encompasses a racemic mixture of the above chiral isomers (i.e., a mixture of the two isomers wherein the ratio of the two isomers is approximately 50:50). And Formula (I) encompasses any other mixture of the above two chiral isomers wherein the ratio of the two isomers is other than approximately 50:50.

In some embodiments, a single chiral isomer corresponding to Formula (I) (or a salt thereof) is obtained by isolating it from a mixture of isomers (or a salt thereof) using, for example, chiral chromatographic separation. In other embodiments, a single chiral isomer of Formula (I) (or a salt thereof) is obtained through direct synthesis from, for example, a chiral starting material. In some embodiments, the ratio of one chiral isomer to its mirror chiral isomer (in, for example, a pharmaceutical composition) is greater than about 9:1. In some such embodiments, the ratio is at least about 95:5. In other such embodiments, the ratio is at least about 98:2. In still yet other such embodiments, the ratio is at least about 99:1. And in still yet other such embodiments, one chiral isomer is present without any detectable amount of its mirror chiral isomer.

When a structure shows the chirality of a carbon, it depicts the direction of one of the chiral carbon's substituents with a dark wedge or hashed wedge, like those shown in the above two Formulas (IA) and (IB), respectively. Unless otherwise indicated, the carbon substituent pointing in the opposite direction is hydrogen. This notation is consistent with conventional organic chemistry nomenclature rules. Thus, for example, Formula (IA) can alternatively be depicted as follows in Formula (IA-1):

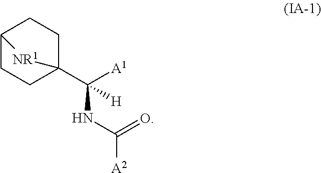

Similarly, Formula (IB) can alternatively be depicted as follows in Formula (IB-1):

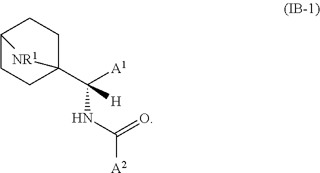

Contemplated salts of the compounds of this invention include both acid addition salts. A salt may be advantageous due to one or more of its chemical or physical properties, such as stability in differing temperatures and humidities, or a desirable solubility in water, oil, or other solvent. In some instances, a salt may be used to aid in the isolation or purification of the compound. In some embodiments (particularly where the salt is intended for administration to an animal, or is a reagent for use in making a compound or salt intended for administration to an animal), the salt is pharmaceutically acceptable.

In general, an acid addition salt can be prepared using various inorganic or organic acids. Such salts can typically be formed by, for example, mixing the compound with an acid (e.g., a stoichiometric amount of acid) using various methods known in the art. This mixing may occur in water, an organic solvent (e.g., ether, ethyl acetate, ethanol, isopropanol, or acetonitrile), or an aqueous/organic mixture. Examples of inorganic acids that typically may be used to form acid addition salts include hydrochloric, hydrobromic, hydroiodic, nitric, carbonic, sulfuric, and phosphoric acid. Examples of organic acids include, for example, aliphatic, cycloaliphatic, aromatic, araliphatic, heterocyclic, carboxylic, and sulfonic classes of organic acids. Specific examples of organic salts include cholate, sorbate, laurate, acetate, trifluoroacetate, formate, propionate, succinate, glycolate, gluconate, digluconate, lactate, malate, tartaric acid (and derivatives thereof, e.g., dibenzoyltartrate), citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate (and derivatives thereof), embonate(pamoate), ethanesulfonate, benzenesulfonate, pantothenate, 2-hydroxyethanesulfonate, sulfanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. In some embodiments, the salt is selected from acetate, benzenesulfonate, benzoate, bicarbonate, bisulfate, bitartrate, borate, bromide, calcium, camsylate, carbonate, chloride, clavulanate, citrate, dihydrochloride, edentate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, myethylsulfate, mutate, napsylate, nitrate, N-methylglucamine ammonium salt, oleate, oxalate, pamoate(embonate), palmitate, pantothenate, phosphate/diphosphate, polygalacturonate, salicylate, stearate, subacetate, succinate, sulfate, sulfonate, tannate, tartrate, teoclate, tosylate, triethiodide, and valerate. In some embodiments, the salt comprises a citric acid salt or a formic acid salt.

The compounds of Formula (I) and salts thereof are intended to encompass any tautomer that may form. A “tautomer” is any other structural isomer that exists in equilibrium resulting from the migration of a hydrogen atom, e.g., amide-imidic acid tautomerism.

It is contemplated that an amine of a compound of Formula (I) or a salt thereof may form an N-oxide. Such an N-oxide is intended to be encompassed by the compounds of Formula (I) and salts thereof. An N-oxide can generally be formed by treating an amine with an oxidizing agent, such as hydrogen peroxide or a per-acid (e.g., a peroxycarboxylic acid). See, e.g., Advanced Organic Chemistry, by Jerry March, 4^{th} Edition, Wiley Interscience. N-oxides also can be made by reacting the amine with m-CPBA, for example, in an inert solvent, such as dichloromethane. See L. W. Deady, Syn. Comm., 7, pp. 509-514 (1977).

It is contemplated that a compound of Formula (I) or salt thereof could form isolatable atropisomer in certain solvents at certain temperatures. The compounds of Formula I and salts thereof are intended to encompass any such atropisomers. Atropisomers can generally be isolated using, for example, chiral LC.

The compounds of Formula (I) and salts thereof are intended to encompass any isotopically-labeled (or “radio-labeled”) derivatives of a compound of Formula (I) or salt thereof. Such a derivative is a derivative of a compound of Formula (I) or salt thereof wherein one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium) ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. The radionuclide that is used will depend on the specific application of that radio-labeled derivative. For example, for in vitro receptor labeling and competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

The compounds of Formula (I) and salts thereof are intended to cover all solid-state forms of the compounds of Formula (I) and salts thereof. The compounds of Formula (I) and salts thereof also are intended to encompass all solvated (e.g., hydrated) and unsolvated forms of the compounds of Formula (I) and salts thereof.

The compounds of Formula (I) and salts thereof also are intended to encompass coupling partners in which a compound of Formula (I) or a salt thereof is linked to a coupling partner by, for example, being chemically coupled to the compound or salt or physically associated with it. Examples of coupling partners include a label or reporter molecule, a supporting substrate, a carrier or transport molecule, an effector, a drug, an antibody, or an inhibitor. Coupling partners can be covalently linked to a compound of Formula (I) or salt thereof via an appropriate functional group on the compound, such as an amino group. Other derivatives include formulating a compound of Formula (I) or a salt thereof with liposomes.

This invention provides, in part, methods to treat various disorders in animals, particularly mammals. Mammals include, for example, humans. Mammals also include, for example, companion animals (e.g., dogs, cats, and horses), livestock animals (e.g., cattle and swine); lab animals (e.g., mice and rats); and wild, zoo, and circus animals (e.g., bears, lions, tigers, apes, and monkeys).

As shown below in the Examples, compounds and salts of this invention have been observed to modulate, and, in particular, act as antagonist against, the glycine transporter 1 (“GlyT1”). Accordingly, it is believed that the compounds and salts of this invention can be used to modulate the glycine transporter to treat various conditions mediated by (or otherwise associated with) the glycine transporter. In some embodiments, the compounds and salts of this invention exhibit one or more of the following characteristics: desirable potency, desirable efficacy, desirable stability on the shelf, desirable tolerability for a range of patients, and desirable safety.

In some embodiments, a compound of Formula (I) or a salt thereof is used to modulate (typically antagonize) GlyT1.

In some embodiments, a compound of Formula (I) or a pharmaceutically acceptable salt thereof is used to treat a condition (typically a disorder) associated with GlyT1 activity.

In some embodiments, a compound of Formula (I) or a pharmaceutically acceptable salt thereof is used to treat a psychosis in a patient in need of such treatment.

In some embodiments, a compound of Formula (I) or a pharmaceutically acceptable salt thereof is used to treat a cognitive disorder in a patient in need of such treatment.

In some embodiments a compound of Formula (I) or a pharmaceutically acceptable salt thereof is used to treat a psychotic disorder.

In some embodiments, for example, a compound of Formula (I) or a pharmaceutically acceptable salt thereof is used to treat schizophrenia.

In some embodiments a compound of Formula (I) or a pharmaceutically acceptable salt thereof is used to treat a schizoaffective disorder.

In some embodiments a compound of Formula (I) or a pharmaceutically acceptable salt thereof is used to treat a delusional disorder.

In some embodiments a compound of Formula (I) or a pharmaceutically acceptable salt thereof is used to treat a brief psychotic disorder.

In some embodiments a compound of Formula (I) or a pharmaceutically acceptable salt thereof is used to treat a shared psychotic disorder.

In some embodiments a compound of Formula (I) or a pharmaceutically acceptable salt thereof is used to treat a psychotic disorder due to a general medical condition.

In some embodiments a compound of Formula (I) or a pharmaceutically acceptable salt thereof is used to treat a mood disorder. Mood disorders include, for example, a) depressive disorders, including but not limited to major depressive disorders and dysthymic disorders; b) bipolar depression and/or bipolar mania including but not limited to bipolar i, including but not limited to those with manic, depressive or mixed episodes, and bipolar ii; c) cyclothymiac's disorders; and d) mood disorders due to a general medical condition.

In some embodiments, a compound of Formula (I) or a pharmaceutically acceptable salt thereof is used to treat a bipolar disorder.

In some embodiments, a compound of Formula (I) or a pharmaceutically acceptable salt thereof is used to treat a cognitive disorder selected from mania and manic depression disorders.

In some embodiments a compound of Formula (I) or a pharmaceutically acceptable salt thereof is used to treat an anxiety disorder. In some such embodiments, the anxiety disorder comprises a disorder selected from a panic disorder without agoraphobia, panic disorder with agoraphobia, agoraphobia without history of any panic disorder, specific phobia, social phobia, an obsessive-compulsive disorder, a stress related disorder, a post-traumatic stress disorder, an acute stress disorder, a generalized anxiety disorder, and a generalized anxiety disorder due to a general medical condition.

In some embodiments a compound of Formula (I) or a pharmaceutically acceptable salt thereof is used to treat a post-traumatic stress disorder.

In some embodiments a compound of Formula (I) or a pharmaceutically acceptable salt thereof is used to treat dementia.

In some embodiments a compound of Formula (I) or a pharmaceutically acceptable salt thereof is used to treat a sleep disorder.

In some embodiments a compound of Formula (I) or a pharmaceutically acceptable salt thereof is used to treat a disorder that is often first diagnosed in infancy, childhood, or adolescence. Such disorders generally include, for example, mental retardation, downs syndrome, learning disorders, motor skills disorders, communication disorders, pervasive developmental disorders, attention-deficit and disruptive behavior disorders, feeding and eating disorders of infancy or early childhood, tic disorders, and elimination disorders.

In some embodiments a compound of Formula (I) or a pharmaceutically acceptable salt thereof is used to treat a substance-related disorder. Such disorders include, for example, substance dependence; substance abuse; substance intoxication; substance withdrawal; alcohol-related disorders; amphetamines (or amphetamine-like)-related disorders; caffeine-related disorders; cannabis-related disorders; cocaine-related disorders; hallucinogen-related disorders; inhalant-related disorders; nicotine-related disorders; opioid-related disorders; phencyclidine (or phencyclidine-like)-related disorders; and sedative-, hypnotic- or anxiolytic-related disorders.

In some embodiments a compound of Formula (I) or a pharmaceutically acceptable salt thereof is used to treat an attention-deficit and disruptive behavior disorder.

In some embodiments a compound of Formula (I) or a pharmaceutically acceptable salt thereof is used to treat an eating disorder.

In some embodiments a compound of Formula (I) or a pharmaceutically acceptable salt thereof is used to treat a personality disorder. Such disorders include, for example, obsessive-compulsive personality disorders.

In some embodiments a compound of Formula (I) or a pharmaceutically acceptable salt thereof is used to treat an impulse-control disorder.

In some embodiments a compound of Formula (I) or a pharmaceutically acceptable salt thereof is used to treat a tic disorder. Such disorders include, for example, Tourette's disorder, chronic motor or vocal tic disorder; and transient tic disorder.

Many of the above conditions and disorder(s) are defined for example in the American Psychiatric Association: diagnostic and statistical manual of mental disorders, fourth edition, text revision, Washington, D.C., American Psychiatric Association, 2000.

It is contemplated that a compound or salt of this invention may be used to treat pain. Such pain may be, for example, chronic pain, neuropathic pain, acute pain, back pain, cancer pain, pain caused by rheumatoid arthritis, migraine, or visceral pain.

It is contemplated that a compound of Formula I or a pharmaceutically acceptable salt thereof may be administered orally, buccally, vaginally, rectally, via inhalation, via insufflation, intranasally, sublingually, topically, or parenterally (e.g., intramuscularly, subcutaneously, intraperitoneally, intrathoracially, intravenously, epidurally, intrathecally, intracerebroventricularly, or by injection into the joints).

In some embodiments, a compound or salt of this invention is administered orally.

In some embodiments, a compound or salt of this invention is administered intravenously.

In some embodiments, a compound or salt of this invention is administered intramuscularly.

In some embodiments, a compound or salt of this invention is used to make a medicament (i.e., a pharmaceutical composition). In general, the pharmaceutical composition comprises a therapeutically effective amount of the compound or salt. Pharmaceutical compositions comprising a compound or salt of this invention can vary widely. Although it is contemplated that a compound or salt of this invention could be administered by itself (i.e., without any other active or inactive ingredient), the pharmaceutical composition normally will instead comprise one or more additional active ingredients and/or inert ingredients. The inert ingredients present in the pharmaceutical compositions of this invention are sometimes collectively referred to as “carriers and diluents.” Methods for making pharmaceutical compositions and the use of carriers and diluents are well known in the art. See, e.g., for example, Remington's Pharmaceutical Sciences, Mack Publishing Company, Easton, Pa., 15th Edition, 1975.

Pharmaceutical compositions comprising a compound of Formula I or pharmaceutically acceptable salt thereof can vary widely. For example, it is contemplated that the compositions may be formulated for a variety of suitable routes and means of administration, including oral, rectal, nasal, topical, buccal, sublingual, vaginal, inhalation, insufflation, or parenteral administration. It is contemplated that such compositions may, for example, be in the form of solids, aqueous or oily solutions, suspensions, emulsions, creams, ointments, mists, gels, nasal sprays, suppositories, finely divided powders, and aerosols or nebulisers for inhalation. In some embodiments, the composition comprises a solid or liquid dosage form that may be administered orally.

Solid form compositions may include, for example, powders, tablets, dispersible granules, capsules, cachets, and suppositories. A solid carrier may comprise one or more substances. Such substances are generally inert. A carrier also may act as, for example, a diluent, flavoring agent, solubilizer, lubricant, preservative, stabilizer, suspending agent, binder, or disintegrating agent. It also may act as, for example, an encapsulating material. Examples of often suitable carriers include pharmaceutical grade mannitol, lactose, magnesium carbonate, magnesium stearate, talc, lactose, sugar (e.g., glucose and sucrose), pectin, dextrin, starch, tragacanth, cellulose, cellulose derivatives (e.g., methyl cellulose and sodium carboxymethyl cellulose), sodium saccharin, low-melting wax, and cocoa butter.

In powders, the carrier is typically a finely divided solid, which is in a mixture with the finely divided active component. In tablets, the active component is typically mixed with the carrier having the desirable binding properties in suitable proportions and compacted into the desired shape and size.

For preparing suppository compositions, a low-melting wax (e.g., a mixture of fatty acid glycerides and cocoa butter) is typically first melted, followed by dispersing the active ingredient therein by, for example, stirring. The molten homogeneous mixture is then poured into convenient-sized molds and allowed to cool and solidify. Examples of non-irritating excipients that may be present in suppository compositions include, for example, cocoa butter, glycerinated gelatin, hydrogenated vegetable oils, mixtures of polyethylene glycols of various molecular weights, and fatty acid esters of polyethylene glycol.

Liquid compositions can be prepared by, for example, dissolving or dispersing the compound or a salt of this invention in a carrier, such as, for example, water, water/propylene glycol solutions, saline aqueous dextrose, glycerol, or ethanol. In some embodiments, aqueous solutions for oral administration can be prepared by dissolving a compound or salt of this invention in water with a solubilizer (e.g., a polyethylene glycol). Colorants, flavoring agents, stabilizers, and thickening agents, for example, also may be added. In some embodiments, aqueous suspensions for oral use can be made by dispersing the compound or salt of this invention in a finely divided form in water, together with a viscous material, such as, for example, one or more natural synthetic gums, resins, methyl cellulose, sodium carboxymethyl cellulose, or other suspending agents. If desired, the liquid composition also may contain other non-toxic auxiliary inert ingredients, such as, for example, wetting or emulsifying agents, pH buffering agents and the like, for example, sodium acetate, sorbitan monolaurate, triethanolamine sodium acetate, sorbitan monolaurate, triethanolamine oleate, etc. Such compositions also may contain other ingredients, such as, for example, one or more pharmaceutical adjuvants.

In some embodiments, the pharmaceutical composition comprises from about 0.05% to about 99% (by weight) of a compound or salt of this invention. In some such embodiments, for example, the pharmaceutical composition comprises from about 0.10% to about 50% (by weight) of a compound or salt of this invention.

When a compound or salt of this invention is administered as a sole therapy for treating a condition (typically a disorder or disease), a “therapeutically effective amount” is an amount sufficient to reduce or completely alleviate symptoms or other detrimental effects of the condition; cure the condition; reverse, completely stop, or slow the progress of the condition; reduce the risk of the condition getting worse; or delay or reduce the risk of onset of the condition.

The optimum dosage and frequency of administration will depend on the particular condition being treated and its severity; the species of the patient; the age, size and weight, diet, and general physical condition of the particular patient; brain/body weight ratio; other medication the patient may be taking; the route of administration; the formulation; and various other factors known to physicians (in the context of human patients), veterinarians (in the context of non-human patients), and others skilled in the art.

It is contemplated that in some embodiments, the optimum amount of a compound or salt of this invention is greater than about 10 pg/kg of body weight per day. In some embodiments, the optimum amount of a compound or salt of this invention is at least about 0.1 mg/kg of body weight per day. In some embodiments, the optimum amount is no greater than about 20 mg/kg of body weight per day. In some embodiments, the optimum amount is from about 0.1 mg/kg to about 20 mg/kg of body weight per day.

It is contemplated that the pharmaceutical compositions can be in one or more unit dosage forms. Accordingly, the composition may be divided into unit doses containing appropriate quantities of the active component. The unit dosage form can be, for example, a capsule, cachet, or tablet itself, or it can be the appropriate number of any of these in packaged forms. The unit dosage form alternatively can be a packaged preparation in which the package contains discrete quantities of the composition, such as, for example, packeted tablets, capsules, or powders in vials or ampoules. Unit dosage forms may be prepared by, for example, various methods well known in the art of pharmacy.

It is contemplated that a dosage can be given once daily or in divided doses, such as, for example, from 2 to 4 times per day.

It is contemplated that a compound of Formula (I) or a salt thereof may be administered concurrently, simultaneously, sequentially, or separately with one or more other pharmaceutically active compounds. It is contemplated that, in some such embodiments, the other pharmaceutically active compound(s) may be one or more other compounds of Formula (I) and/or pharmaceutically acceptable salts thereof. It also is contemplated that, in some embodiments, the other pharmaceutically active compound(s) may be selected from one or more of the following: antidepressants; antipsychotics; anxiolytics; anticonvulsants; Alzheimer's therapies; Parkinson's therapies; agents for treating extrapyramidal symptoms; migraine therapies; stroke therapies; neuropathic pain therapies; nociceptive pain therapies; insomnia therapies; mood stabilizers; agents for treating ADHD; agents used to treat substance abuse disorders, dependence, and withdrawal; a cognitive enhancing agent; a memory enhancing agent; an anti-inflammatory agent; and a selective serotonin reuptake inhibitor (or “serotonin-specific reuptake inhibitor” or SSRI”). It is also contemplated that a compound of Formula (I) or salt thereof may be administered as part of a combination therapy with radiotherapy. In addition, it is contemplated that a compound of Formula (I) or salt thereof may be administered as a combination therapy with chemotherapy. In some such embodiments, the chemotherapy includes one or more of the following categories of anti-tumor agents: antiproliferative/antineoplastic drugs, cytostatic agents, anti-invasion agents, inhibitors of growth factor function, antiangiogenic agents, vascular damaging agents, endothelin receptor antagonists, antisense therapies, gene therapy approaches, and immunotherapy approaches. It also is contemplated that a compound of Formula (I) or salt thereof may be useful as an analgesic agent for use during general anesthesia or monitored anesthesia care. Combinations of agents with different properties are often used to achieve a balance of effects needed to maintain the anesthetic state (e.g., amnesia, analgesia, muscle relaxation, and sedation). Such a combination may include, for example, one or more inhaled anesthetics, hypnotics, anxiolytics, neuromuscular blockers, and/or opioids.

In some embodiments in which a combination therapy is used, the amount of a compound of Formula (I) or a salt thereof and the amount of the other pharmaceutically active agent(s) are, when combined, therapeutically effective to treat a targeted disorder in the animal patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; reduce the risk of the disorder getting worse; or delay or reduce the risk of onset of the disorder. Typically, such amounts may be determined by one skilled in the art by, for example, starting with the dosage range described in this patent for a compound of Formula (I) or a salt thereof and an approved or otherwise published dosage range(s) of the other pharmaceutically active compound(s).

When used in a combination therapy, it is contemplated that a compound of Formula (I) or a salt thereof and the other active ingredients may be administered in a single composition, completely separate compositions, or a combination thereof. It also is contemplated that the active ingredients may be administered concurrently, simultaneously, sequentially, or separately. The particular composition(s) and dosing frequency(ies) of the combination therapy will depend on a variety of factors, including, for example, the route of administration, the condition being treated, the species of the patient, any potential interactions between the active ingredients when combined into a single composition, any interactions between the active ingredients when they are administered to the animal patient, and various other factors known to physicians (in the context of human patients), veterinarians (in the context of non-human patients), and others skilled in the art.

This invention also is directed, in part, to a kit comprising a compound of Formula (I) or a salt thereof. In some embodiments, the kit further comprises one or more additional components, such as, for example: (a) an apparatus for administering the compound of Formula (I) or salt thereof (b) instructions for administering the compound of Formula (I) or salt thereof (c) a carrier, diluent, or excipient (e.g., a re-suspending agent); and (d) an additional active ingredient, which may be in the same and/or different dosage forms as the compound of Formula (I) or salt thereof. In some embodiments (particularly when the kit is intended for use in administering the compound of Formula I or salt thereof to an animal patient), the salt is a pharmaceutically acceptable salt.

### EXAMPLES

The following examples are merely illustrative of embodiments of the invention, and not limiting to the remainder of this disclosure in any way.

### A. [3H] Glycine Uptake Assay

### Reagents

Preparation of recombinant human GlyT1b-CHO cells (hGlyT1b-CHO). The human GlyT1b CDS (GC002087, NM_{—}006934) was cloned downstream of a CMV promoter in a bicistronic expression vector containing a hygromycin B resistance gene. CHO-K1 cells (ATCC) were transfected with the recombinant vector containing GlyT1b using Lipofectamine 2000 (Invitrogen) and cultured in Ham's/F12 media supplemented with 10% fetal bovine serum, 2 mM L-glutamine at 37° C., 5% CO₂, 90% humidity. Twenty-four hours after transfection, cells were diluted and switched to media containing 0.5 mg/ml hygromycin B. Antibiotic resistant cells were obtained after 21 days of culture in the presence of hygromycin B. Clonal stable cell lines were isolated by FACS single cell deposition into 96-well plates. Clonal cell lines were assessed for GlyT1b expression by measuring uptake of ³H-glycine and the clone showing the highest uptake was selected for the development of the glycine uptake assay.

Cell culture:

Cells used were Recombinant hGlyT1b/CHO. These cells were cultured in cell culture medium (Ham's/F12 (Modified) (Mediatech, 10-080-CM), containing 10% FBS, 2 mM L-glutamine (Invitrogen 25030-149) and 0.5 mg/mL hygromycin B (Invitrogen, 10687-010)) in 175 cm² flasks until near confluence before use in the assay.

Cell suspension:

Cell medium in a cell culture flask containing near confluent cells was removed and 5 mL of cell stripper was added to submerge all cells on the surface of the culture flask. Cell stripper was removed immediately and the flask incubated in a 37° C. incubator for ˜5 min. Cells were shaken loose and suspended in 5 mL of PBS. After splitting cells to initiate a new flask(s), the cells remaining were collected by centrifugation, counted, and resuspended in assay buffer to a density of ˜2 million/mL. The cell suspension was kept at room temperature before use. The assays buffer was 10 mM HEPES, pH 7.4, containing 150 mM NaCl, 5 mM KCl, 1.5 mM CaCl₂, 1.5 mM MgCl₂, 0.45 mg/mL L-alanine (added fresh), and 1.8 mg/mL D-glucose (added fresh).

SPA and isotope mixture:

WGA PTV beads were suspended in assay buffer (2 mg/ml) containing 60 nM [³H]Glycine (PerkinElmer (NET-004, [2-³H]Glycine, 53.3 Ci/mmol, 1 mCi/mL)) and 20 μM unlabeled glycine and the suspension was kept at room temperature before assay.

Assay of glycine uptake:

To the wells of an OptiPlate, 2 μl DMSO containing a test compound was spotted. This was followed by addition of 98 μl of cell suspension (˜1 million/ml final). After incubating cells with compound for ˜15 min, 100 μl of the SPA (200 μg/well final) and isotope mixture (30 nM isotope with 10 μM cold glycine, final) was added to initiate the glycine uptake. At 2 h, the plate was read on a TopCount to quantify SPA counts.

### B. HPLC Analysis

The IC chiral supercritical fluid chromatography (SFC) column was obtained from Chiral Technologies, West Chester, Pa.

Mass Spectroscopy Method MS-1
Instrumentation: Waters Acquity SQD
Ionization mode: Electrospray
Column: Acquity UPLC BEH C18 2.1×50 mm×1.7 um
Mobile phase A: Water:Acetonitrile:Formic acid (98:2:0.1 v/v)
Mobile Phase B: Water:Acetonitrile:Formic acid (2:98:0.05 v/v)
Gradient: Time (% B): 0(5); 0.9(95); 1.2(95); 1.3(5); 1.4(5)

Mass Spectroscopy Method MS-2
Instrumentation: Waters ZMD fronted with an Agilent 1100 LC
Ionization mode: APCI
Column: Zorbax SB-C8 2.1×50 mm×5 um
Column temp: Ambient
Mobile phase A: Water:Acetonitrile:Formic acid (98:2:0.1 v/v)
Mobile Phase B: Water:Acetonitrile:Formic acid (2:98:0.05 v/v)
Flow Rate: 1.4 ml/min (split)
Gradient: Time (% B): 0(5); 3(90); 4(90); 4.5(5); 5(5)

Mass Spectroscopy Method MS-3
Instrumentation: Waters Acquity SQD
Ionization mode: Electrospray
Column: Acquity UPLC BEH C 18 2.1×50 mm×1.7 um
Column temp: 55° C.
Mobile phase A: Water:Methanol:Formic acid (98:2:0.1 v/v)
Mobile Phase B: Water:Methanol:Formic acid (2:98:0.05 v/v)
Flow rate: 0.9 ml/min (split)
Gradient: Time (% B): 0(5); 0.9(95); 1.5(95); 1.6(5); 1.9(5)

Mass Spectroscopy Method MS4
Instrumentation: Waters ZMD fronted with an Agilent 1100 LC
Ionization mode: APCI
Column: Zorbax SB-C8 2.1×50 mm×5 um
Column temp: Ambient
Mobile phase A: Water:Methanol:Formic acid (98:2:0.1 v/v)
Mobile Phase B: Water:Methanol:Formic acid (2:98:0.05 v/v)
Flow Rate: 1 ml/min (split)
Gradient: Time (% B): 0(5); 2.5(95); 4(95); 4.2(5); 5(5)
   C. Illustrative Compounds of this Invention and their [³H]Glycine Uptake Assay Results

The examples below illustrate a variety of different compounds of this invention. The examples also provide a variety of generic schemes for preparing compounds of this invention, as well as specific examples illustrating those schemes. It is expected that one skilled in the art of organic synthesis, after reading these examples alone or in combination with the general knowledge in the art, can adapt and apply the methods to make any compound encompassed by this invention. The general knowledge in the art includes, for example:
i) Conventional procedures for using protective groups and examples of suitable protective groups, which are described in, for example, Protective Groups in Organic Synthesis, T. W. Green, P. G. M. Wuts, Wiley-Interscience, New York (1999).
ii) References discussing various organic synthesis reactions, include textbooks of organic chemistry, such as, for example, Advanced Organic Chemistry, March 4th ed, McGraw Hill (1992); and Organic Synthesis, Smith, McGraw Hill, (1994). They also include, for example, R. C. Larock, Comprehensive Organic Transformations, 2nd ed., Wiley-VCH: New York (1999); F. A. Carey; R. J. Sundberg, Advanced Organic Chemistry, 2nd ed., Plenum Press: New York (1984); L. S. Hegedus, Transition Metals in the Synthesis of Complex Organic Molecules, 2nd ed., University Science Books: Mill Valley, Calif. (1994); L. A. Paquette, Ed., The Encyclopedia of Reagents for Organic Synthesis, John Wiley: New York (1994); A. R. Katritzky, O. Meth-Cohn, C W. Rees, Eds., Comprehensive Organic Functional Group Transformations, Pergamon Press: Oxford, UK (1995); G. Wilkinson; F. G A. Stone; E. W. Abel, Eds., Comprehensive Organometallic Chemistry, Pergamon Press: Oxford, UK (1982); B. M. Trost; I. Fleming, Comprehensive Organic Synthesis, Pergamon Press: Oxford, UK (1991); A. R. Katritzky, C W. Rees Eds., Comprehensive Heterocyclic Chemistry, Pergamon Press: Oxford, UK (1984); A. R. Katritzky; C W. Rees, E. F. V. Scriven, Eds., Comprehensive Heterocyclic Chemistry II, Pergamon Press: Oxford, UK (1996); C. Hansen; P. G. Sammes; J. B. Taylor, Eds., Comprehensive Medicinal Chemistry: Pergamon Press: Oxford, UK (1990). In addition, recurring reviews of synthetic methodology and related topics include: Organic Reactions, John Wiley: New York; Organic Syntheses; John Wiley: New York; The Total Synthesis of Natural Products, John Wiley: New York; The Organic Chemistry of Drug Synthesis, John Wiley: New York; Annual Reports in Organic Synthesis, Academic Press: San Diego Calif.; and Methoden der Organischen Chemie (Houben-Weyl), Thieme: Stuttgart, Germany.
iii) References discussing heterocyclic chemistry include, for example, example, Heterocyclic Chemistry, J. A. Joule, K. Mills, G. F. Smith, 3rd ed., Cheapman and Hall, p. 189-225 (1995); and Heterocyclic Chemistry, T. L. Gilchrist, 2^{nd} ed. Longman Scientific and Technical, p. 248-282 (1992).
iv) Databases of synthetic transformations, including Chemical Abstracts, which may be searched using either CAS Online or SciFinder; and Handbuch der Organischen Chemie (Beilstein), which may be searched using SpotFire.

### Method 1. Stereoselective Synthesis of N—H Azabicyclo[2.2.1]heptanes

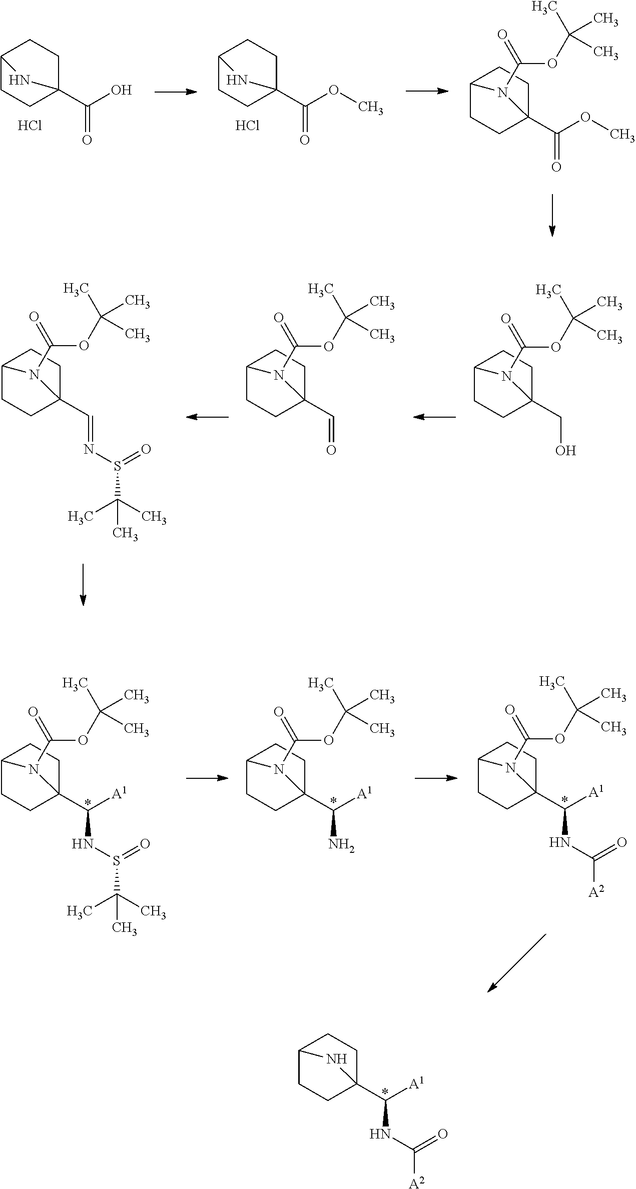

Method 1 depicts a generalized scheme suitable for stereoselective synthesis of N—H azabicyclo[2.2.1]heptanes. Those skilled in the art will readily recognize various reagents and intermediates or changes in moieties that could be used to make additional N—H azabicyclo[2.2.1]heptanes, either stereoselectively or in racemic form.

### Example 1

### Preparation of (R*)—N-(7-azabicyclo[2.2.1]heptan-1-yl(phenyl)methyl)-2,6-dimethylbenzamide

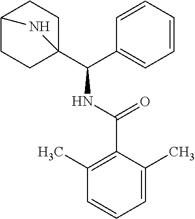

### Step A. Preparation of 7-tert-butyl 1-methyl 7-azabicyclo[2.2.1]heptane-1,7-dicarboxylate from (1s,4s)-7-azabicyclo[2.2.1]heptane-1-carboxylic acid hydrochloride

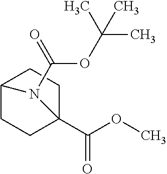

To methanol (80 mL) at 0° C. was added acetyl chloride (3.90 mL, 54.89 mmol) slowly. After 10 min, this solution was added to (1s,4s)-7-azabicyclo[2.2.1]heptane-1-carboxylic acid (3.25 g, 18.30 mmol; prepared according to the procedures of A. Avenoza et al. Tetrahedron 2001, 57, 545-548) to afford a beige mixture. The mixture was warmed to 60° C. and maintained at these conditions for 16 h. The mixture was concentrated to minimal volume, reconcentrated from methanol, and dried under vacuum to afford crude (1s,4s)-methyl 7-azabicyclo[2.2.1]heptane-1-carboxylate (3.46 g) as the hydrochloride salt and a light gray solid. To a mixture of crude methyl 7-azabicyclo[2.2.1]heptane-1-carboxylate hydrochloride (2.0 g, 10.44 mmol), triethylamine (7.27 mL, 52.18 mmol) and dichloromethane (50 mL) was added di-tert-butyl dicarbonate (2.91 mL, 12.52 mmol). The resulting white mixture was stirred at room temperature for 16 h and was then diluted with saturated aqueous sodium bicarbonate. The layers were separated and the aqueous layer was extracted with ethyl acetate (×3). The combined organic layers were dried over sodium sulfate, filtered and concentrated. The resulting residue was purified by flash column chromatography (SiO₂, 0-50% ethyl acetate in hexanes) to afford 7-tert-butyl 1-methyl 7-azabicyclo[2.2.1]heptane-1,7-dicarboxylate (2.050 g, 77%) as a clear colorless oil. 1H NMR (300 MHz, chloroform-d) δ ppm 1.41 (s, 9H), 1.43-1.53 (m, 2H), 1.68-1.80 (m, 2H), 1.85-2.00 (m, 2H), 2.11-2.26 (m, 2H), 3.79 (s, 3H), 4.33 (t, J=4.8 Hz, 1H). m/z (ES+), (M+Na)+=278.1.

### Step B. Preparation of tert-butyl 1-(hydroxymethyl)-7-azabicyclo[2.2.1]heptane-7-carboxylate from 7-tert-butyl 1-methyl 7-azabicyclo[2.2.1]heptane-1,7-dicarboxylate

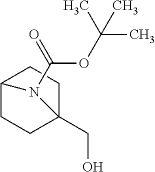

To a solution of 7-tert-butyl 1-methyl 7-azabicyclo[2.2.1]heptane-1,7-dicarboxylate (0.78 g, 3.04 mmol) in tetrahydrofuran (9.41 mL) at room temperature was added 1.0 M diisobutylaluminum hydride in toluene (6.38 mL, 6.38 mmol), resulting in an exotherm. After 30 min, the reaction was quenched with 1N aqueous hydrogen chloride and then basified with 50% aqueous sodium hydroxide. The resulting mixture was extracted with ethyl acetate (×3). The combined organic layers were dried over sodium sulfate, filtered, and concentrated. The resulting residue was purified by flash column chromatography (SiO₂, 5-10% ethyl acetate in dichloromethane, visualization with PMA) to afford semi-pure tert-butyl 1-(hydroxymethyl)-7-azabicyclo[2.2.1]heptane-7-carboxylate (0.665 g, 106%) as a clear colorless free-flowing oil. 1H NMR (300 MHz, chloroform-d) δ ppm 1.32-1.52 (m, 4H), 1.44-1.46 (m, 9H), 1.70-1.96 (m, 4H), 3.90 (d, J=7.2 Hz, 2H), 4.24 (t, J=4.5 Hz, 1H), 4.78 (br. s., 1H). m/z (ES+), (M-tBu+2H)+=172.0.

### Step C. Preparation of tert-butyl 1-formyl-7-azabicyclo[2.2.1]heptane-7-carboxylate from tert-butyl 1-(hydroxymethyl)-7-azabicyclo[2.2.1]heptane-7-carboxylate

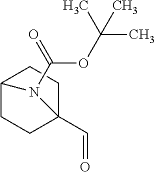

To a solution of DMSO (2.00 mL, 28.27 mmol) in dichloromethane (35 mL) at −78° C. was added dropwise oxalyl chloride (1.24 mL, 14.13 mmol). After stirring the resulting mixture vigorously for 15 min, to the now-clear solution was added tert-butyl 1-(hydroxymethyl)-7-azabicyclo[2.2.1]heptane-7-carboxylate (1.29 g, 5.65 mmol) as a solution in dichloromethane (10 mL) via syringe. The reaction became cloudy and opaque and was maintained at −78° C. for 30 min. Then, triethylamine (7.88 mL, 56.53 mmol) was added in one portion and the white mixture was maintained at −78° C. for another 10 min before being warmed to 0° C. After another 10 min, the reaction was quenched with saturated aqueous sodium bicarbonate, and the layers were separated. The aqueous layer was extracted with ethyl acetate (×2), and the combined organic layers were dried over sodium sulfate, filtered, and concentrated. The resulting residue was purified by flash column chromatography (SiO₂, 0-10% ethyl acetate in hexanes, visualization with PMA) to afford tert-butyl 1-formyl-7-azabicyclo[2.2.1]heptane-7-carboxylate (1.15 g, 90%) as a clear colorless free-flowing oil. 1H NMR (300 MHz, chloroform-d) δ ppm 1.43 (s, 9H), 1.46-1.70 (m, 4H), 1.83-2.09 (m, 4H), 4.23-4.37 (m, 1H), 9.92 (s, 1H). m/z (ES+), (M-tBu+2H)+=170.1.

### Step D. Preparation of (R)-tert-butyl 1-((tert-butylsulfinylimino)methyl)-7-azabicyclo[2.2.1]heptane-7-carboxylate from tert-butyl 1-formyl-7-azabicyclo[2.2.1]heptane-7-carboxylate

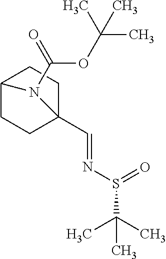

To a light yellow solution of tert-butyl 1-formyl-7-azabicyclo[2.2.1]heptane-7-carboxylate (1.15 g, 5.10 mmol) and tetraethoxytitanium (2.52 mL, 10.21 mmol) in tetrahydrofuran (10.24 mL) was added (R)-2-methylpropane-2-sulfinamide (0.650 g, 5.36 mmol). The resulting solution was stirred at room temperature for 16 h and then eight drops of saturated aqueous sodium bicarbonate were added. The resulting mixture was diluted with ethyl acetate (10 mL), stirred vigorously for 25 min and then filtered. The filtrate was concentrated and the resulting residue was purified by flash column chromatography (SiO₂, 0-40% ethyl acetate in hexanes) to afford (R)-tert-butyl 1-((tert-butylsulfinylimino)methyl)-7-azabicyclo[2.2.1]heptane-7-carboxylate (1.41 g, 84%) as a white semi-crystalline solid. 1H NMR (300 MHz, chloroform-d) δ ppm 1.20 (s, 9H), 1.41 (s, 9H), 1.44-1.65 (m, 3H), 1.70-1.83 (m, 1H), 1.85-2.13 (m, 4H), 4.33 (t, J=4.6 Hz, 1H), 8.51 (s, 1H). m/z (ES+), (M+H)+=329.2.

### Step E. Preparation of tert-butyl 1-((R*)—((R)-1,1-dimethylethylsulfinamido)(phenyl)methyl)-7-azabicyclo[2.2.1]-heptane-7-carboxylate and tert-butyl 1-((S*)—((R)-1,1-dimethylethylsulfinamido)(phenyl)methyl)-7-azabicyclo[2.2.1]-heptane-7-carboxylate from (R)-tert-butyl 1-((tert-butylsulfinylimino)methyl)-7-azabicyclo[2.2.1]heptane-7-carboxylate

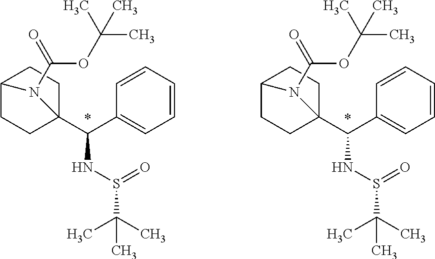

To a solution of (R)-tert-butyl 1-((tert-butylsulfinylimino)methyl)-7-azabicyclo[2.2.1]heptane-7-carboxylate (1.10 g, 3.35 mmol) in tetrahydrofuran (14.33 mL) at −78° C. was added dropwise 1.8 M phenyllithium in di-n-butyl ether (2.42 mL, 4.35 mmol), maintaining a reaction temperature below −70° C. After 10 min, the reaction was quenched with saturated aqueous sodium chloride. The mixture was then extracted with ethyl acetate (×3), and the combined organic layers were dried over sodium sulfate, filtered, and concentrated. The resulting residue was purified by flash column chromatography (SiO₂, 0-25% ethyl acetate in hexanes, then 25% isocratic ethyl acetate in hexanes, then 50% isocratic ethyl acetate in hexanes) to afford the faster eluting diastereomer of tert-butyl 1-(((R)-1,1-dimethylethylsulfinamido)(phenyl)methyl)-7-azabicyclo[2.2.1]heptane-7-carboxylate (1.25 g, 92%) as a clear colorless oil containing a small amount of ethyl acetate and the slower eluting diastereomer of tert-butyl 1-(((R)-1,1-dimethylethylsulfinamido)(phenyl)methyl)-7-azabicyclo[2.2.1]heptane-7-carboxylate (0.198 g, 15%) as a clear colorless residue containing a small amount of ethyl acetate. The faster eluting (major) diastereomer was arbitrarily assigned as the (R*,R) diastereomer, and the slower eluting (minor) diastereomer was arbitrarily assigned as the (S*,R) diastereomer. tert-butyl 1-((R*)—((R)-1,1-dimethylethylsulfinamido)(phenyl)methyl)-7-azabicyclo[2.2.1]-heptane-7-carboxylate: 1H NMR (300 MHz, chloroform-d) δ ppm 1.08-1.18 (m, 1H), 1.24 (s, 9H), 1.25-1.31 (m, 2H), 1.31-1.42 (m, 1H), 1.51 (s, 9H), 1.69-1.87 (m, 3H), 2.20-2.32 (m, 1H), 4.32 (t, J=4.8 Hz, 1H), 5.20-5.28 (m, 2H), 7.27 (d, J=1.9 Hz, 3H), 7.34-7.39 (m, 2H). m/z (ES+), (M+H)+=407.3; MS-1, HPLC tR=1.02 min. tert-butyl 1-((S*)—((R)-1,1-dimethylethylsulfinamido)(phenyl)methyl)-7-azabicyclo[2.2.1]-heptane-7-carboxylate: 1H NMR (300 MHz, chloroform-d) δ ppm 1.13-1.20 (m, 1H), 1.22 (s, 9H), 1.25-1.44 (m, 3H), 1.47 (s, 9H), 1.65-1.89 (m, 3H), 2.18-2.33 (m, 1H), 4.27 (t, J=4.8 Hz, 1H), 5.13-5.27 (m, 2H), 7.23-7.36 (m, 3H), 7.44-7.50 (m, 2H). m/z (ES+), (M+H)+=407.3; MS-1, HPLC tR=0.98 min.

### Step F. Preparation (R*)-tert-butyl 1-(amino(phenyl)methyl)-7-azabicyclo[2.2.1]heptane-7-carboxylate from tert-butyl 1-((R*)—((R)-1,1-dimethylethylsulfinamido)(phenyl)methyl)-7-azabicyclo[2.2.1]-heptane-7-carboxylate

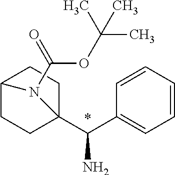

To a solution of tert-butyl 1-((R*)—((R)-1,1-dimethylethylsulfinamido)(phenyl)methyl)-7-azabicyclo[2.2.1]-heptane-7-carboxylate (1.25 g, 3.07 mmol) in methanol (28.1 mL) at 0° C. was added 4 M hydrochloric acid in dioxane (2.69 mL, 10.76 mmol). After 30 min, the reaction was warmed to room temperature and stirred for 20 min. Then the reaction was quenched with saturated aqueous sodium bicarbonate, extracted with ethyl acetate (×3), and the combined organic layers were dried over sodium sulfate, filtered, and concentrated to afford crude (R*)-tert-butyl 1-(amino(phenyl)methyl)-7-azabicyclo[2.2.1]heptane-7-carboxylate (0.92 g, 99%) as a light yellow viscous oil. 1H NMR (300 MHz, chloroform-d) δ ppm 1.00 (ddd, J=11.8, 9.5, 4.8 Hz, 1H), 1.15-1.29 (m, 4H), 1.32-1.42 (m, 1H), 1.49 (s, 9H), 1.62-1.87 (m, 3H), 2.40 (tt, J=12.1, 3.8 Hz, 1H), 4.26 (t, J=4.8 Hz, 1H), 5.00 (s, 1H), 7.22-7.32 (m, 3H), 7.36-7.46 (m, 2H). m/z (ES+), (M+H)+=303.2.

### Step G. Preparation of (R*)-tert-butyl 1-((2,6-dimethylbenzamido)(phenyl)methyl)-7-azabicyclo[2.2.1]heptane-7-carboxylate from (R*)-tert-butyl 1-(amino(phenyl)methyl)-7-azabicyclo[2.2.1]heptane-7-carboxylate

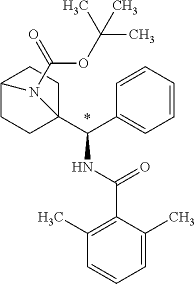

To a solution of 2,6-dimethylbenzoic acid (0.114 g, 0.76 mmol) in dichloromethane (2 mL) was added oxalyl chloride (0.133 mL, 1.52 mmol) followed by 1 drop of DMF. After 2 h, the solution was concentrated to an oily semi-solid, redissolved in dichloromethane and reconcentrated to a light gold oil. This oil was then added via syringe as a solution in dichloromethane (1 mL) to a solution of (R*)-tert-butyl 1-(amino(phenyl)methyl)-7-azabicyclo[2.2.1]heptane-7-carboxylate (0.046 g, 0.15 mmol) and DIPEA (0.213 mL, 1.22 mmol) also in dichloromethane (1.18 mL). After 4.5 h, the reaction was concentrated to minimal volume and stored in a freezer for 16 h. The reaction was then purified by flash column chromatography (SiO₂, 0-100% ethyl acetate in hexanes) to afford (R*)-tert-butyl 1-((2,6-dimethylbenzamido)(phenyl)methyl)-7-azabicyclo[2.2.1]heptane-7-carboxylate (0.052 g, 79%) as a clear colorless residue. 1H NMR (300 MHz, chloroform-d) δ ppm 1.24-1.36 (m, 2H), 1.43 (s, 9H), 1.47-1.56 (m, 1H), 1.59-1.73 (m, 2H), 1.73-1.89 (m, 2H), 2.14 (td, J=8.2, 3.8 Hz, 1H), 2.21 (s, 6H), 4.30 (t, J=4.8 Hz, 1H), 5.86 (d, J=8.6 Hz, 1H), 6.96 (d, J=7.6 Hz, 2H), 7.04-7.15 (m, 1H), 7.20-7.35 (m, 3H), 7.54 (dd, J=8.1, 1.4 Hz, 2H), 8.15 (d, J=8.0 Hz, 1H). m/z (ES+), (M+H)+=435.3.

### Step H. Preparation of (R*)—N-(7-azabicyclo[2.2.1]heptan-1-yl(phenyl)methyl)-2,6-dimethylbenzamide from (R*)-tert-butyl 1-((2,6-dimethylbenzamido)(phenyl)methyl)-7-azabicyclo[2.2.1]heptane-7-carboxylate

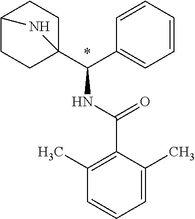

To (R*)-tert-butyl 1-((2,6-dimethylbenzamido)(phenyl)methyl)-7-azabicyclo[2.2.1]-heptane-7-carboxylate (0.052 g, 0.12 mmol) was added 12 N aqueous hydrochloric acid (1.0 mL, 12.00 mmol). After bubbling ceased (˜1 min), the mixture was basified with saturated aqueous sodium bicarbonate and extracted with ethyl acetate (×3). The combined organic layers were dried over sodium sulfate, filtered, and concentrated to afford crude product. This material was dissolved in methanol, filtered a second time, and purified by preparative HPLC (C18, acetonitrile in water containing ammonium carbonate, pH 10) to afford (R*)—N-(7-azabicyclo[2.2.1]heptan-1-yl(phenyl)methyl)-2,6-dimethylbenzamide (0.040 g, 100%) as a white foam solid. 1H NMR (300 MHz, chloroform-d) δ ppm 1.18-1.34 (m, 1H), 1.38-1.50 (m, 5H), 1.60-1.74 (m, 1H), 1.75-1.90 (m, 1H), 2.25 (s, 6H), 3.49-3.61 (m, 1H), 5.42 (d, J=8.0 Hz, 1H), 6.83 (d, J=7.8 Hz, 1H), 6.94-7.06 (m, 2H), 7.14 (dd, J=8.2, 7.2 Hz, 1H), 7.27-7.33 (m, 1H), 7.35 (d, J=4.2 Hz, 4H). m/z (ES+), (M+H)+=335.2; MS-1, HPLC tR=0.48 min.

### Method 2. Stereoselective Synthesis of N-Me Azabicyclo[2.2.1]heptanes

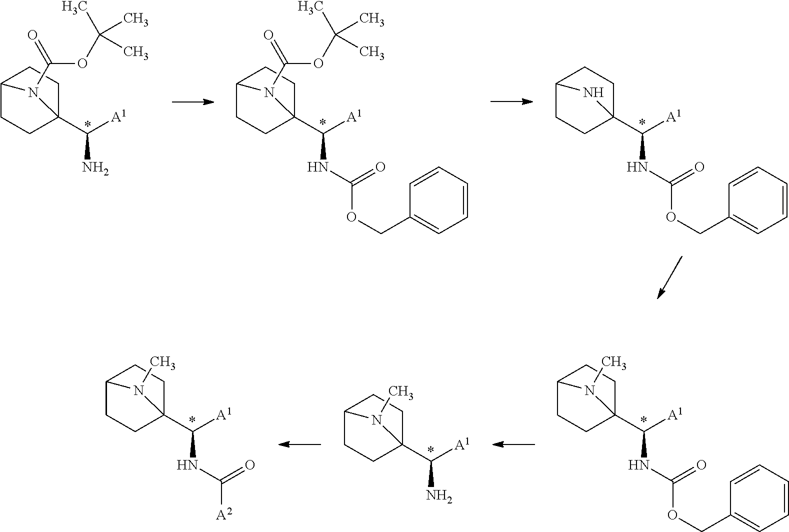

Method 2 depicts a generalized scheme suitable for stereoselective synthesis of N-Me azabicyclo[2.2.1]heptanes. Those skilled in the art will readily recognize various reagents and intermediates or changes in moieties that could be used to make additional N-alkyl azabicyclo[2.2. 1]heptanes.

### Example 2

### Preparation of (R*)—N-((7-methyl-7-azabicyclo[2.2.1]heptan-1-y)(phenyl)methyl)-2-(methylthio)nicotinamide

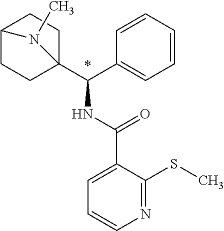

### Step A. Preparation of (R*)-tert-butyl 1-((benzyloxycarbonylamino)(phenyl)methyl)-7-azabicyclo[2.2.1]heptane-7-carboxylate from (R*)-tert-butyl 1-(amino(phenyl)methyl)-7-azabicyclo[2.2.1]heptane-7-carboxylate

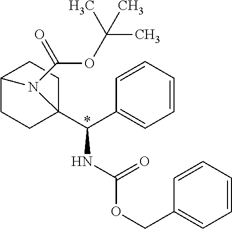

To a solution of (R*)-tert-butyl 1-(amino(phenyl)methyl)-7-azabicyclo[2.2.1]heptane-7-carboxylate (0.133 g, 0.44 mmol; prepared according to the procedures of Example 1, Steps A-F) and DIPEA (0.230 mL, 1.32 mmol) in dichloromethane (4.09 mL) was added benzyl chloroformate (0.073 mL, 0.48 mmol). The resulting light yellow solution was stirred for 20 min and another 35 uL of benzyl chloroformate were added. The reaction was stirred for another 45 min before being quenched with methanol (1 mL) and concentrated to minimal volume. The resulting solution was purified by flash column chromatography (SiO₂, 0-30% ethyl acetate in hexanes) to afford (R*)-tert-butyl 1-((benzyloxycarbonylamino)(phenyl)methyl)-7-azabicyclo[2.2.1]heptane-7-carboxylate (0.180 g, 94%) as a clear colorless oil. 1H NMR (300 MHz, chloroform-d) δ ppm 1.23-1.34 (m, 2H), 1.45 (s, 9H), 1.45-1.50 (m, 2H), 1.62-1.72 (m, 1H), 1.76-1.91 (m, 2H), 1.91-2.02 (m, 1H), 4.30 (t, J=4.8 Hz, 1H), 4.70 (d, J=5.9 Hz, 1H), 4.99 (d, J=12.4 Hz, 1H), 5.12 (d, J=12.4 Hz, 1H), 5.34 (d, J=7.0 Hz, 1H), 7.20-7.34 (m, 6H), 7.36 (d, J=4.2 Hz, 2H), 7.43 (d, J=7.0 Hz, 2H). m/z (ES+), (M+H)+=437.3.

### Step B. Preparation of (R*)-benzyl 7-azabicyclo[2.2.1]heptan-1-yl(phenyl)methylcarbamate from (R)-tert-butyl 1-((benzyloxycarbonylamino)(phenyl)methyl)-7-azabicyclo[2.2.1]heptane-7-carboxylate

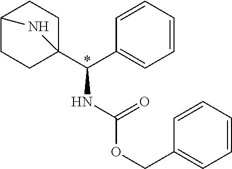

To (R*)-tert-butyl 1-((benzyloxycarbonylamino)(phenyl)methyl)-7-azabicyclo[2.2.1]heptane-7-carboxylate (0.180 g, 0.41 mmol) was added 12N aqueous hydrochloric acid (1.0 mL, 12.00 mmol) followed by methanol (0.5 mL) and dichloromethane (0.5 mL). After 5 min of stirring, the mixture was concentrated until it became clear. Another 1 mL of aqueous hydrochloric acid (12 M) was added followed by 1 mL of methanol and the solution was again concentrated to minimal volume. The mixture was then basified with saturated aqueous sodium bicarbonate and extracted with ethyl acetate (×3). The combined organic layers were dried over sodium sulfate, filtered, and concentrated to afford crude (R*)-benzyl 7-azabicyclo[2.2.1]heptan-1-yl(phenyl)methylcarbamate (0.141 g, 102%) as a light yellow oil. 1H NMR (500 MHz, chloroform-d) δ ppm 1.30-1.42 (m, 3H), 1.42-1.50 (m, 2H), 1.50-1.59 (m, 1H), 1.62-1.74 (m, 2H), 3.55-3.60 (m, 1H), 4.92-5.12 (m, 3H), 6.01 (br. s., 1H), 7.23-7.39 (m, 10H). m/z (ES+), (M+H)+=337.2.

### Step C. Preparation of (R*)-benzyl(7-methyl-7-azabicyclo[2.2.1]heptan-1-yl)(phenyl)methylcarbamate from (R*)-benzyl 7-azabicyclo[2.2.1]heptan-1-yl(phenyl)methylcarbamate

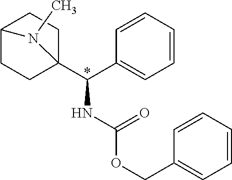

To (R*)-benzyl 7-azabicyclo[2.2.1]heptan-1-yl(phenyl)methylcarbamate (0.268 g, 0.80 mmol) was added 37 wt % aqueous formaldehyde (1.5 mL, 20.15 mmol) and formic acid (3.0 mL, 78.22 mmol). The resulting solution was sealed and warmed to 60° C. After 16 h, the reaction was transferred to a microwave vial and subjected to microwave conditions for 60 min (300 W, 125° C.). The reaction was resubjected to the same conditions for another 30 min before being basified with saturated aqueous ammonium hydroxide. The mixture was extracted with ethyl acetate (×3), and the combined organic layers were dried over sodium sulfate, filtered, and concentrated to a light yellow oil. The resulting oil was purified by flash column chromatography (SiO₂, 0-20% methanol in ethyl acetate) to afford (R*)-benzyl(7-methyl-7-azabicyclo[2.2.1]heptan-1-yl)(phenyl)methylcarbamate (0.186 g, 66.6%) as a clear colorless oil. 1H NMR (300 MHz, chloroform-d) δ ppm 0.94-1.06 (m, 1H), 1.07-1.22 (m, 2H), 1.30-1.43 (m, 1H), 1.58-1.82 (m, 3H), 1.91-2.04 (m, 1H), 2.22 (s, 3H), 3.22 (t, J=4.5 Hz, 1H), 4.71 (d, J=4.0 Hz, 1H), 4.93-5.13 (m, 2H), 5.86 (br. s., 1H), 7.07-7.46 (m, 10H). m/z (ES+), (M+H)+=351.2.

### Step D. Preparation of (R*)—N-((7-methyl-7-azabicyclo[2.2.1]heptan-1-yl)(phenyl)methyl)-2-(methylthio)nicotinamide from (R*)-benzyl(7-methyl-7-azabicyclo[2.2.1]heptan-1-yl)(phenyl)methylcarbamate

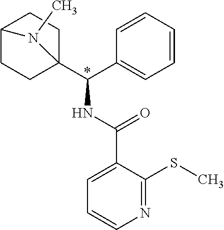

To a vacuum degassed solution of (R*)-benzyl(7-methyl-7-azabicyclo[2.2.1]heptan-1-yl)(phenyl)methylcarbamate (0.048 g, 0.14 mmol) in methanol (1.370 mL) was added 20 wt % palladium hydroxide on carbon (0.020 g, 0.03 mmol). The reaction flask was then equipped with a hydrogen balloon (1 atm), and the reaction mixture was stirred vigorously for 2.5 days. The mixture was then filtered, and the filtrate was concentrated to afford crude (R*)-(7-methyl-7-azabicyclo[2.2.1]heptan-1-yl)(phenyl)methanamine (0.033 g, 111%) of an estimated 80% purity as a cloudy residue. m/z (ES+), (M+H)+=217.1. To crude (R*)-(7-methyl-7-azabicyclo[2.2.1]heptan-1-yl)(phenyl)methanamine (0.0328 g, 0.12 mmol) in DMF (1.149 mL) was added DIPEA (0.064 mL, 0.36 mmol), 2-(methylthio)nicotinic acid (0.025 g, 0.15 mmol), HOBT (0.022 g, 0.15 mmol), and TBTU (0.047 g, 0.15 mmol) sequentially. The light beige reaction gradually became yellow, and, after 1.5 h, was filtered and purified via preparative HPLC (C 18, acetonitrile in water containing ammonium carbonate, pH 10) to afford (R*)—N-((7-methyl-7-azabicyclo[2.2.1]heptan-1-yl)(phenyl)methyl)-2-(methylthio)nicotinamide (0.017 g, 38.4%) as a white solid upon lyopholization. 1H NMR (300 MHz, chloroform-d) δ ppm 1.12 (dd, J=11.0, 3.6 Hz, 1H), 1.16-1.32 (m, 2H), 1.35-1.48 (m, 1H), 1.63-2.09 (m, 4H), 2.24 (s, 3H), 2.60 (s, 3H), 3.26 (t, J=4.6 Hz, 1H), 5.10 (d, J=4.6 Hz, 1H), 7.04 (dd, J=7.6, 4.8 Hz, 1H), 7.19-7.36 (m, 3H), 7.42 (d, J=7.4 Hz, 3H), 7.88 (dd, J=7.6, 1.7 Hz, 1H), 8.50 (dd, J=4.8, 1.7 Hz, 1H). m/z (ES+), (M+H)+=368.2.

### Method 3. Racemic Synthesis of N-Me Azabicyclo[2.2.1]heptanes

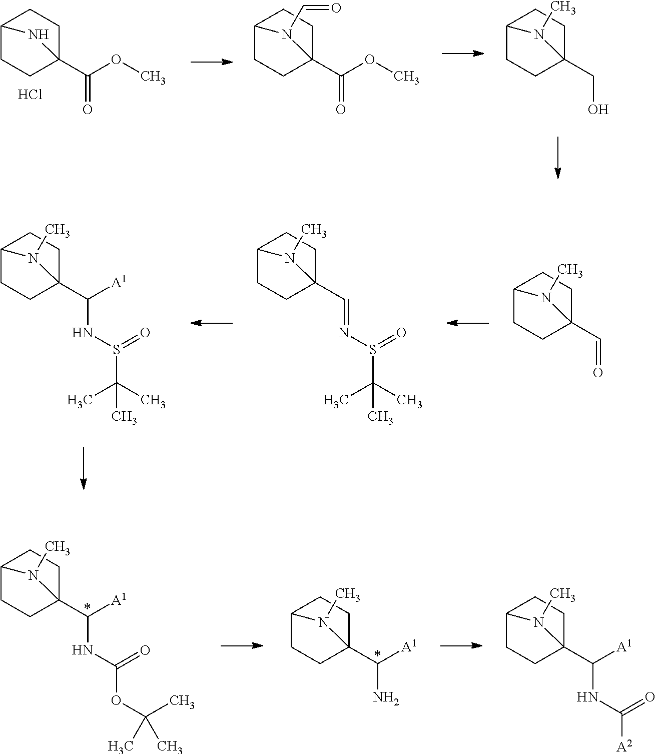

Method 3 depicts a generalized scheme suitable for racemic synthesis of N-Me azabicyclo[2.2.1]heptanes. Those skilled in the art will readily recognize various reagents and intermediates or changes in moieties that could be used to make additional N-alkyl azabicyclo[2.2.1]heptanes.

### Example 3

### Preparation of 2,6-dimethyl-N-((7-methyl-7-azabicyclo[2.2.1]heptan-1-yl)(phenyl)methyl)benzamide

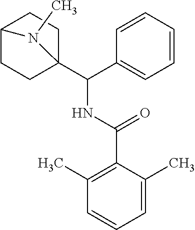

### Step A. Preparation of methyl 7-formyl-7-azabicyclo[2.2.1]heptane-1-carboxylate from (1s,4s)-methyl 7-azabicyclo[2.2.1]heptane-1-carboxylate hydrochloride

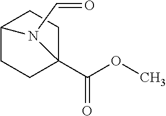

To acetic anhydride (1.596 mL, 16.92 mmol) at 0° C. was added formic acid (0.757 mL, 17.63 mmol). After 5 min, the clear colorless solution was warmed to 60° C. After 1 h, the solution was cooled, and 0.5 mL were added to a mixture of triethylamine (9.83 mL, 70.50 mmol) and (1s,4s)-methyl 7-azabicyclo[2.2.1]heptane-1-carboxylate, hydrochloride (2.70 g, 14.1 mmol; prepared according to the procedures of A. Avenoza et al. Tetrahedron 2001, 57, 545-548) in dichloromethane (70 mL) at 0° C. After 10 min, the white mixture was was diluted with saturated aqueous sodium bicarboante and extracted with ethyl acetate (x3). The combined organic layers were dried over sodium sulfate, filtered, and concentrated. The resulting residue was purified by flash column chromatography (SiO₂, 0-100% ethyl acetate) to afford (1s,4s)-methyl 7-formyl-7-azabicyclo[2.2.1]heptane-1-carboxylate (1.70 g, 65.8%) as a clear light yellow oil. 1H NMR (300 MHz, chloroform-d) δ ppm 1.49-1.68 (m, 2H), 1.74-1.99 (m, 4H), 1.99-2.32 (m, 2H), 3.84 (s, 3H), 4.15-4.32 (m, 0.26H), 4.79 (br. s., 0.74H), 8.10-8.28 (m, 0.26H), 8.39 (br. s., 0.74H). m/z (ES+), (M+H)+=184.1.

### Step B. Preparation of (7-methyl-7-azabicyclo[2.2.1]heptan-1-yl)methanol from methyl 7-formyl-7-azabicyclo[2.2.1]heptane-1-carboxylate

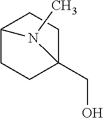

To a solution of concentrated aqueous sulfuric acid (1.61 mL, 30.16 mmol) in tetrahydrofuran (80 mL) at 0° C. was added 2.0 M lithium aluminum hydride in tetrahydrofuran (30.2 mL, 60.32 mmol) dropwise. After 15 min, methyl 7-formyl-7-azabicyclo[2.2.1]heptane-1-carboxylate (1.7 g, 9.28 mmol) was added via cannula as a solution in tetrahydofuran (10 mL). After 3 min, the reaction was warmed to room temperature. After another 30 min, the reaction was re-cooled to 0° C. and quenched with ethyl acetate and then sodium sulfate decahydrate. The mixture was stirred vigorously for 15 min and filtered. The filtrate was then concentrated, and the crude residue was treated with ether. The resulting white mixture was filtered again and the filtrate was concentrated to afford crude (7-methyl-7-azabicyclo[2.2.1]heptan-1-yl)methanol (0.687 g, 52.4%) as a white oily residue. 1H NMR (300 MHz, chloroform-d) δ ppm 1.23-1.45 (m, 4H), 1.57-1.93 (m, 5H), 2.17 (s, 3H), 3.23 (t, J=4.6 Hz, 1H), 3.73 (br. s., 2H). m/z (ES+), (M+H)+=172.16.

### Step C. Preparation of 7-methyl-7-azabicyclo[2.2.1]heptane-1-carbaldehyde from (7-methyl-7-azabicyclo[2.2.1]heptan-1-yl)methanol

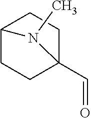

To a solution of oxalyl chloride (0.639 mL, 7.30 mmol) in dichloromethane (15 mL) was added DMSO (0.691 mL, 9.73 mmol) dropwise at -78° C. After 7 min, a solution of (7-methyl-7-azabicyclo[2.2.1]heptan-1-yl)methanol (0.687 g, 4.87 mmol) in dichloromethane (3 mL) was added via cannula. After 15 min, triethylamine (3.39 mL, 24.33 mmol) was added in one portion. After another 15 min, the white mixture was warmed to room temperature over 30 min and then quenched with saturated aqueous sodium bicarbonate. The layers were separated, and the aqueous layer was extracted with ethyl acetate (×2). The combined organic layers were dried over sodium sulfate, filtered and concentrated. The resulting residue was treated with ethyl acetate (5 mL), and the resulting mixture was filtered. The filtrate was concentrated to afford crude 7-methyl-7-azabicyclo[2.2.1]heptane-1-carbaldehyde (0.342 g, 50.5%) as a light yellow oil. 1H NMR (300 MHz, chloroform-d) δ ppm 1.38-1.58 (m, 4H), 1.84-2.06 (m, 4H), 2.24 (s, 3H), 3.34 (t, J=4.2 Hz, 1H), 9.94 (s, 1H). m/z (ES+), (M+MeOH+H)+=172.2.

### Step D. Preparation of 2-methyl-N-((7-methyl-7-azabicyclo[2.2.1]heptan-1-yl)methylene)propane-2-sulfinamide from 7-methyl-7-azabicyclo[2.2.1]heptane-1-carbaldehyde

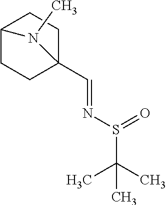

To a solution of 7-methyl-7-azabicyclo[2.2.1]heptane-1-carbaldehyde (0.342 g, 2.46 mmol) and tetraethoxytitanium (0.927 mL, 4.42 mmol) in tetrahydrofuran (6.14 mL) was added 2-methylpropane-2-sulfinamide (0.357 g, 2.95 mmol). After 20 h, the reaction was quenched by the dropwise addition of saturated aqueous sodium bicarbonate (1.5 mL) and diluted with ethyl acetate (6 mL). The resulting yellow mixture was vigorously stirred for 30 min and then filtered. The filtrate was concentrated and the resulting yellow residue was purified by flash column chromatography (SiO₂, 100% ethyl acetate, then 20% methanol in ethyl acetate) to afford 2-methyl-N-((7-methyl-7-azabicyclo[2.2.1]heptan-1-yl)methylene)propane-2-sulfinamide (0.247 g, 41.5%) as a clear colorless oil. 1H NMR (300 MHz, chloroform-d) δ ppm 1.21 (s, 9H), 1.39-1.50 (m, 2H), 1.54-1.67 (m, 2H), 1.85-2.07 (m, 4H), 2.23 (s, 3H), 3.33-3.38 (m, 1H), 8.29 (s, 1H). m/z (ES+), (M+H)+=243.2.

### Step E. Preparation of tert-butyl(7-methyl-7-azabicyclo[2.2.1]heptan-1-yl)(phenyl)methylcarbamate from 2-methyl-N-((7-methyl-7-azabicyclo[2.2.1]heptan-1-yl)methylene)propane-2-sulfinamide

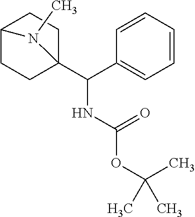

To a solution of 2-methyl-N-((7-methyl-7-azabicyclo[2.2.1]heptan-1-yl)methylene)propane-2-sulfinamide (0.247 g, 1.02 mmol) in tetrahydrofuran (0.5 mL) was added 1.0 M phenylmagnesium bromide in tetrahydrofuran (4.08 mL, 4.08 mmol), resulting in an orange-red solution. After 15 min, the reaction was quenched with 50% saturated aqueous ammonium chloride in saturated aqueous ammonium hydroxide. The mixture was diluted with ethyl acetate and the layers were separated. The aqueous layer was extracted with ethyl acetate (×2), and the combined organic layers were dried over sodium sulfate, filtered and concentrated to afford crude 2-methyl-N-((7-methyl-7-azabicyclo[2.2.1]heptan-1-yl)(phenyl)methyl)propane-2-sulfinamide. To a solution of crude 2-methyl-N-((7-methyl-7-azabicyclo[2.2.1]heptan-1-yl)(phenyl)methyl)propane-2-sulfinamide from above in methanol (2.0 mL) was added 4M hydrochloric acid in dioxane (3 mL). After 5 min, the light orange solution was concentrated, and saturated aqueous sodium bicarbonate (2 mL) was added followed by ethyl acetate (2 mL). To this mixture was added di-tert-butyl dicarbonate (0.474 mL, 2.04 mmol) in one portion. After 30 min, the mixture was extracted with ethyl acetate (×3), and the combined organic layers were dried over sodium sulfate, filtered, and concentrated. The filtrate was concentrated, and the residue was purified by flash column chromatography (SiO₂, 100% ethyl acetate for 5 min, then 20% methanol in ethyl acetate for 25 min). Approximately 70 mg of desired product was obtained as a clear residue (see below). To the aqueous layer from the above extraction (following Boc protection) was added di-tert-butyl dicarbonate (0.474 mL, 2.04 mmol) and tetrahydrofuran (10 mL). The resulting mixture was vigorously stirred for 60 min and then extracted with ethyl acetate (×3). The combined organic layers were dried over sodium sulfate, filtered, and concentrated. The resulting residue was purified via flash column chromatography as above, and the resulting product was combined with the aforementioned 70 mg to provide tert-butyl (7-methyl-7-azabicyclo[2.2.1]heptan-1-yl)(phenyl)methylcarbamate (0.262 g, 81%) as a clear viscous oil. 1H NMR (300 MHz, chloroform-d) δ ppm 0.93-1.07 (m, 1H), 1.12-1.42 (m, 3H), 1.38 (br. s., 9H), 1.68-1.90 (m, 3H), 1.92-2.08 (m, 1H), 2.34 (s, 3H), 3.32 (br. s., 1H), 4.68 (br. s., 0H), 5.67 (br. s., 1H), 7.18-7.37 (m, 5H). m/z (ES+), (M+H)+=317.2.

### Step F. Preparation of (7-methyl-7-azabicyclo[2.2.1]heptan-1-yl)(phenyl)methanamine bis hydrochloride from tert-butyl (7-methyl-7-azabicyclo[2.2.1]heptan-1-yl)(phenyl)methylcarbamate

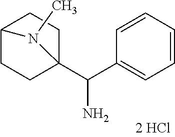

To tert-butyl (7-methyl-7-azabicyclo[2.2.1]heptan-1-yl)(phenyl)methylcarbamate (0.262 g, 0.83 mmol) was added concentrated aqueous hydrogen chloride (1.2 mL). After gas evolution ceased, the solution was concentrated to a glass and reconcentrated from methanol and dichloromethane to afford (7-methyl-7-azabicyclo[2.2.1]heptan-1-yl)(phenyl)methanamine bishydrochloride (0.224 g, 107%; contains a small amount of methanol) as a mixture of diastereomers and a white foam solid. 1H NMR (300 MHz, MeOD) δ ppm 1.37 (ddd, J=13.7, 9.7, 4.2 Hz, 1H), 1.81 (ddd, J=13.6, 9.8, 4.1 Hz, 1H), 1.87-2.08 (m, 2H), 2.09-2.26 (m, 2H), 2.27-2.43 (m, 1H), 2.55-2.68 (m, 1H), 2.96 (s, 3H), 4.03-4.25 (m, 1H), 4.90-5.13 (m, 1H), 7.44-7.64 (m, 5H). m/z (ES+), (M−H-2Cl)+=217.2.

### Step G. Preparation of 2,6-dimethyl-N-((7-methyl-7-azabicyclo[2.2.1]heptan-1-yl)(phenyl)methyl)benzamide from (7-methyl-7-azabicyclo[2.2.1]heptan-1-yl)(phenyl)methanamine bis hydrochloride

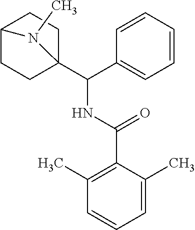

A solution of (7-methyl-7-azabicyclo[2.2.1]heptan-1-yl)(phenyl)methanamine bis hydrochloride (0.021 g, 0.07 mmol), 2,6-dimethylbenzoic acid (0.012 g, 0.08 mmol), and HOBT (0.016 g, 0.11 mmol) in DMF (0.484 mL) was treated with TBTU (0.033 g, 0.10 mmol) and DIPEA (0.126 mL, 0.73 mmol) sequentially. After 1.5 h, the solution was diluted with methanol, filtered, and purified by preparative HPLC (C18, acetonitrile in water containing ammonium carbonate, pH 10) to afford 2,6-dimethyl-N-((7-methyl-7-azabicyclo[2.2.1]heptan-1-yl)(phenyl)methyl)benzamide (0.017 g, 68.8%) as a white foam solid. Alternatively, this material could be prepared by reacting (7-methyl-7-azabicyclo[2.2.1]heptan-1-yl)(phenyl)methanamine bis hydrochloride with 2,6-dimethylbenzoyl chloride in the presence of DIPEA. 1H NMR (300 MHz, chloroform-d) δ ppm 1.02-1.23 (m, 3H), 1.29-1.43 (m, 1H), 1.55-1.84 (m, 3H), 1.90-2.07 (m, 1H), 2.27 (s, 3H), 2.34 (s, 6H), 3.21 (t, J=4.5 Hz, 1H), 5.11 (d, J=5.0 Hz, 1H), 6.62 (br. s., 1H), 7.01 (d, 2H), 7.15 (dd, J=8.0, 7.1 Hz, 1H), 7.21-7.41 (m, 5H). m/z (ES+), (M+H)+=349.3; MS-1, HPLC tR=0.51 min.

### Method 4. Preparation of Compounds of Formula I by Chiral Resolution of a Final Product

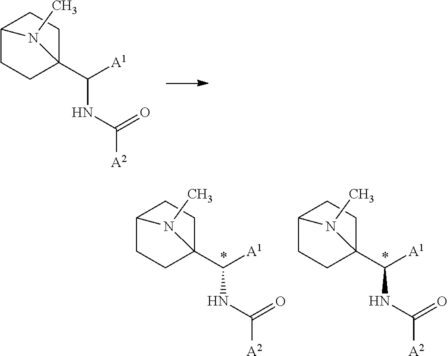

Method 4 depicts a generalized scheme suitable for preparation of compounds of Formula I by chiral resolution of a final product. Those of skill in the art will readily recognize various reagents and intermediates or changes in moieties that could be used to make additional compounds of Formula I.

### Examples 4 and 5

### Preparation (R*)-2,6-dimethyl-N-((7-methyl-7-azabicyclo[2.2.1]heptan-1-yl)(phenyl)methyl)benzamide citric acid salt and (S*)-2,6-dimethyl-N-((7-methyl-7-azabicyclo[2.2.1]heptan-1-yl)(phenyl)methyl)-benzamide citric acid salt

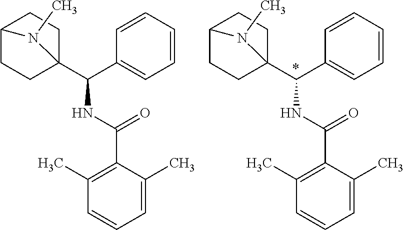

Racemic 2,6-dimethyl-N-((7-methyl-7-azabicyclo[2.2.1]heptan-1-yl)(phenyl)methyl)-benzamide was resolved under supercritical fluid chromatography conditions (liquid CO₂) on a ChiralPak IC column using 25% methanol containing 0.5% dimethylethylamine to afford faster eluting (S*)-2,6-dimethyl-N-47-methyl-7-azabicyclo[2.2.1]heptan-1-yl)(phenyl)methyl)benzamide and slower eluting (R*)-2,6-dimethyl-N-((7-methyl-7-azabicyclo[2.2.1]heptan-1-yl)(phenyl)methyl)benzamide. These compounds were dissolved in 10% methanol in dichloromethane, treated with 1.0 equiv of citric acid monohydrate in methanol and concentrated. The resulting residues were lyopholized to afford (S*)-2,6-dimethyl-N-((7-methyl-7-azabicyclo[2.2.1]heptan-1-yl)(phenyl)methyl)benzamide citric acid salt and (R*)-2,6-dimethyl-N-((7-methyl-7-azabicyclo[2.2.1]heptan-1-yl)(phenyl)methyl)benzamide citric acid salt as white solids. Relative Stereochemistry: In general, the absolute stereochemistry of individual isomers obtained in this manner was not determined. Arbitrary designations were used (R*,S*). (R*)-2,6-dimethyl-N-((7-methyl-7-azabicyclo[2.2.1]heptan-1-yl)(phenyl)methyl)benzamide citric acid salt. 1H NMR (500 MHz, MeOD) δ ppm 1.45-1.55 (m, 1H), 1.68-1.84 (m, 3H), 2.05-2.29 (m, 9H), 2.49-2.59 (m, 1H), 2.63-2.75 (m, 4H), 2.90-3.02 (m, 3H), 4.00 (br. s., 1H), 5.67 (br. s., 1H), 7.02 (d, J=7.6 Hz, 2H), 7.16 (t, J=7.6 Hz, 1H), 7.34-7.47 (m, 3H), 7.50 (d, J=1.5 Hz, 2H). m/z (ES+), (M+H)+=349.3; MS-1, HPLC tR=0.51 min. (S*)-2,6-dimethyl-N-((7-methyl-7-azabicyclo[2.2.1]heptan-1-yl)(phenyl)methyl)benzamide citric acid salt. 1H NMR (500 MHz, MeOD) δ ppm 1.44-1.53 (m, 1H), 1.65-1.84 (m, 3H), 2.06-2.29 (m, 9H), 2.49-2.59 (m, 1H), 2.58-2.70 (m, 4H), 2.93 (br. s., 3H), 3.99 (br. s., 1H), 5.66 (br. s., 1H), 7.02 (d, J=7.6 Hz, 2H), 7.16 (t, J=7.6 Hz, 1H), 7.34-7.47 (m, 3H), 7.49 (d, J=7.0 Hz, 2H). m/z (ES+), (M+H)+=349.3; MS-1, HPLC tR=0.51 min.

### Method 5. Preparation and SFC Resolution of Racemic N-Alkyl Azabicyclo[2.2.1]heptanes

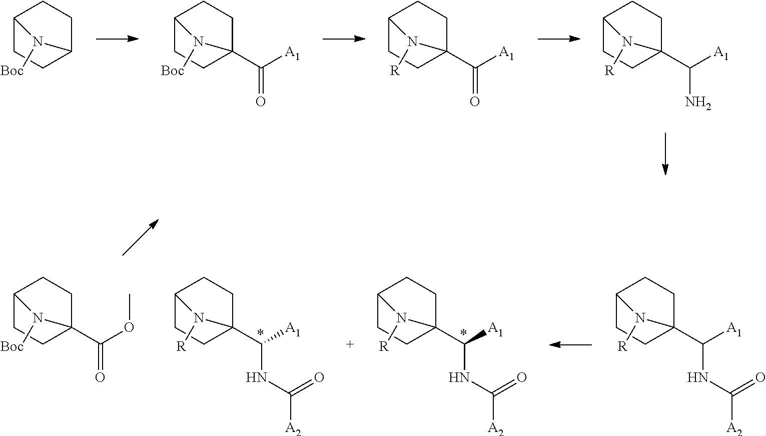

Method 5 depicts a generalized scheme suitable for racemic synthesis of N-alkyl azabicyclo[2.2.1]heptanes. Those skilled in the art will readily recognize various reagents and intermediates or changes in moieties that could be used to make additional N-alkyl azabicyclo[2.2.1]heptanes. The racemic compounds could either be tested directly or could be readily resolved by Super critical-Fluid Chromatography under suitable conditions.

### Example 6

### (R*)—N-((7-(2-methoxyethyl)-7-azabicyclo[2.2.1]heptan-1-yl)(pyridin-4-yl)methyl)-2,6-dimethylbenzamide

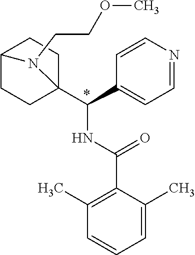

### Step A. Preparation of tert-butyl 1-isonicotinoyl-7-azabicyclo[2.2.1]heptane-7-carboxylate from tert-butyl 7-azabicyclo[2.2.1]heptane-7-carboxylate

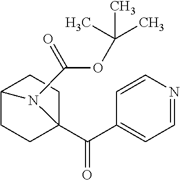

To a stirred solution of tert-butyl 7-azabicyclo[2.2.1]heptane-7-carboxylate (1.068 g, 5.42 mmol) in Et₂O (10 mL), TMEDA (1.137 mL, 7.58 mmol) was added. The mixture was stirred at 0° C. for 15 min, before the dropwise addition of s-BuLi 1.4 M in cyclohexane (4.64 mL, 6.50 mmol). The reaction was stirred at room temperature for 5 min, before the addition of N-methoxy-N-methylisonicotinamide (0.6 g, 3.61 mmol) in 5 mL ether at 0° C. The mixture was then stirred from 0° C. to room temperature for 2 hr. The reaction is quenched with water, The organic layer was separated. The aqueous layer was extracted with ether. The ether layers were combined and washed with brine, dried over MgSO₄, filtered and concentrated. The residue was purified by silica gel column (40 g, 20%-75% Hex/EA) to give tert-butyl 1-isonicotinoyl-7-azabicyclo[2.2.1]heptane-7-carboxylate (0.54 g, 50%). 1HNMR (500 MHz, CDCl3) δ ppm 1.16 (bs, 9H), 1.58-1.63 (m, 2H), 1.82 (bs, 2H), 2.04 (bs, 2H), 2.20-2.27 (m, 2H), 4.48 (s, 1H), 8.06 (d, J=6.0 Hz, 2H), 8.76 (d, J=6.0 Hz, 2H).

### Step B. Preparation of 7-azabicyclo[2.2.1]heptan-1-yl(pyridin-4-yl)methanone from tert-butyl 1-isonicotinoyl-7-azabicyclo[2.2.1]heptane-7-carboxylate

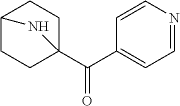

To a stirred solution of tert-butyl 1-isonicotinoyl-7-azabicyclo[2.2.1]heptane-7-carboxylate (550 mg, 1.82 mmol) in 1,4-dioxane (10 mL), 4N HCl (9.09 mL, 36.38 mmol) in dioxane was added. The mixture was stirred at room temperature overnight. The crude product was neutralized with 1N NaOH and extracted with DCM. The extract was dried over MgSO₄, filtered and concentrated to give 7-azabicyclo[2.2.1]heptan-1-yl(pyridin-4-yl)methanone (400 mg, quantitative) as an orange oil. 1HNMR (500 MHz, CDCl3) δ ppm 1.58-1.94 (m, 9H), 3.83 (t, J=4.5 Hz, 1H), 7.95 (d, J=6.0 Hz, 2H), 8.76 (d, J=6.0 Hz, 2H).

### Step C. Preparation of (7-(2-methoxyethyl)-7-azabicyclo[2.2.1]heptan-1-yl)(pyridin-4-yl)methanone from 7-azabicyclo[2.2.1]heptan-1-yl(pyridin-4-yl)methanone

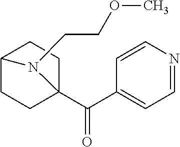

To a mixture of DIPEA (1.036 mL, 5.93 mmol) and 7-azabicyclo[2.2.1]heptan-1-yl(pyridin-4-yl)methanone (400 mg, 1.98 mmol) in DMF (5 mL), 1-bromo-2-methoxyethane (289 mg, 1.98 mmol) was added. The mixture was heated to 150° C. for 15 min in microwave. DMF was removed under reduced pressure. The residue was extracted with ether and 0.5N NaOH. The ether layer was then washed with brine, dried over MgSO₄, filtered and concentrated to give a black oil, which was purified by silica gel (0-10% MeOH in DCM) to give (7-(2-methoxyethyl)-7-azabicyclo[2.2.1]heptan-1-yl)(pyridin-4-yl)methanone (190 mg, 37%) as an orange oil. 1H NMR (500 MHz, CDCl₃) δ ppm 1.49 (dd, J=11.4, 3.8 Hz, 2H), 1.62 (dd, J=9.0, 3.5 Hz, 2H), 1.99 (dd, J=10.1, 4.9 Hz, 2H), 2.10-2.20 (m, 2H), 2.43 (t, J=5.8 Hz, 2H), 3.20 (s, 3H), 3.38 (t, J=5.8 Hz, 2H), 3.69 (t, J=4.7 Hz, 1H), 8.29 (d, J=6.1 Hz, 2H), 8.78 (d, J=5.8 Hz, 2H).

### Step D. Preparation of (7-(2-methoxyethyl)-7-azabicyclo[2.2.1]heptan-1-yl)(pyridin-4-yl)methanamine from 7(7-(2-methoxyethyl)-7-azabicyclo[2.2.1]heptan-1-yl)(pyridin-4-yl)methanone

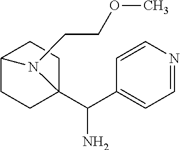

The mixture of 7N ammonia in methanol (9.88 mL, 69.14 mmol), Ti(Oi-Pr)₄ (0.588 mL, 2.01 mmol) and (7-(2-methoxyethyl)-7-azabicyclo[2.2.1]heptan-1-yl)(pyridin-4-yl)methanone (180 mg, 0.69 mmol) was heated to 55° C. overnight in a sealed tube. Then it was cool to room temperature and sodium borohydride (52.3 mg, 1.38 mmol) was added as a solid, and stirred at room temperature for 1 hr. Several ml of 1N NaOH was added, followed by several spatula of celite. After 30 min, the solution was filtered through a pad of celite, and washed with plenty of MeOH. The solution was concentrated, and diluted with water, extracted with DCM (2×20 mL). The DCM layer was then washed with brine, dried over MgSO₄, filtered, and concentrated to give the crude (7-(2-methoxyethyl)-7-azabicyclo[2.2.1]heptan-1-yl)(pyridin-4-yl)methanamine (140 mg, 77%) as a yellow oil. 1HNMR (500 MHz, CDCl₃) δ ppm 0.76-0.82 (m, 1H), 1.02-1.08 (m, 1H), 1.13-1.19 (m, 1H), 1.32-1.39 (m, 1H), 1.50-1.75 (m, 4H), 1.89-1.95 (m, 1H), 2.05-2.10 (m, 1H), 2.40-2.44 (m, 1H), 2.73-2.76 (m, 1H), 3.40 (s, 3H), 3.41-3.43 (m, 1H), 3.52-3.56 (m, 2H), 4.18 (s, 1H), 7.34 (d, J=6.0 Hz, 2H), 8.51 (d, J=6.0 Hz, 2H).

### Step E. Preparation of (R*)—N-((7-(2-methoxyethyl)-7-azabicyclo[2.2.1]heptan-1-yl)(pyridin-4-yl)methyl)-2,6-dimethylbenzamide from (7-(2-methoxyethyl)-7-azabicyclo[2.2.1]heptan-1-yl)(pyridin-4-yl)methanamine

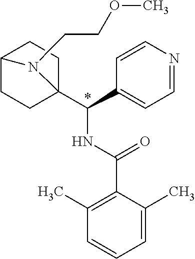

To a stirred solution of 2,6-dimethylbenzoic acid (44.2 mg, 0.29 mmol), DIPEA (0.140 mL, 0.80 mmol) in DCM (5 mL), TBTU (95 mg, 0.29 mmol) was added. After 10 min, (7-(2-methoxyethyl)-7-azabicyclo[2.2.1]heptan-1-yl)(pyridin-4-yl)methanamine (70 mg, 0.27 mmol) was added. The mixture was stirred at room temperature overnight. LCMS showed formation of active ester, but no trace of desired product. The reaction mixture was concentrated and several ml DMF was added, the mixture was heated at 80° C. for 6-8 hr. The reaction is then concentrated and diluted with DCM, and washed with 1N NaOH. The DCM layer was then dried over MgSO₄, filtered, and concentrated. The residue was purified by silica gel column (12 g, 0-10% MeOH in DCM), followed by basic alumina column (0-100% Hex/EA) to provide N-((7-(2-methoxyethyl)-7-azabicyclo[2.2.1]heptan-1-yl)(pyridin-4-yl)methyl)-2,6-dimethylbenzamide (30.0 mg, 28.5%) as a white solid, which was resolved by SFC under these conditions: The Multigram III SFC system was used with a 21 mm×250 mm Chiral ADHcolumn. The sample were diluted in 5 ml of EtOH (0.5% isopropylamine), and stacked injections of 0.8 ml each were run using 20% of MeOH [0.5% isopropylamine] isocratic at 50 ml/min. The ee of sample was check by SFC under similar SFC condition. SFC: peak2: (cc>95%, by SFC, tR=6.95 min); 1H NMR (500 MHz, CDCl3) δ ppm 0.97-1.08 (m, 1H), 1.17-1.30 (m, 2H), 1.39 (m, 1H), 1.57-1.77 (m, 3H), 1.93-2.04 (m, 1H), 2.35 (s, 6H), 2.40-2.52 (m, 1H), 2.65-2.77 (m, 1H), 3.25 (s, 3H), 3.41-3.54 (m, 3H), 5.06 (d, J=4.0 Hz, 1H), 6.87 (br. s., 1H), 7.03 (d, J=7.6 Hz, 2H), 7.17 (t, J=7.6 Hz, 1H), 7.32 (d, J=5.8 Hz, 2H), 8.57 (d, J=5.8 Hz, 2H). m/z (ES+), (M+H)+=394.4; MS-3, HPLC tR=0.52 min.

### Example 7

### Preparation of 2-fluoro-6-methyl-N-((7-methyl-7-azabicyclo[2.2.1]heptan-1-yl)(phenyl)methyl)benzamide

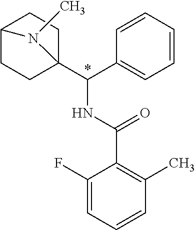

### Step A. Preparation of 7-azabicyclo[2.2.1]heptan-1-yl(phenyl)methanone hydrochloride from -tert-butyl 1-methyl 7-azabicyclo[2.2.1]heptane-1,7-dicarboxylate

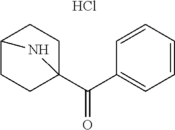

To a stirred solution of 7-tert-butyl 1-methyl 7-azabicyclo[2.2.1]heptane-1,7-dicarboxylate (2 g, 7.83 mmol) in 10 ml THF at −78° C., phenyllithium (10.01 mL, 18.02 mmol) was added dropwise. After stirred at −78° C. for 2 hr, The reaction is quenched with 5 ml 1N HCl at −78° C. The reaction is warmed to room temperature and extracted with EtOAc several times, the organic layers were combined and washed with brine, dried over MgSO₄, filtered and concentrated. The residue was purified by silica gel column, (0-50% Hex/EA). To give tert-butyl 1-benzoyl-7-azabicyclo[2.2.1]heptane-7-carboxylate (2.5 g, quantitative yield). To a stirred solution of tert-butyl 1-benzoyl-7-azabicyclo[2.2.1]heptane-7-carboxylate (2.5 g, 8.30 mmol) in 1,4-dioxane (15 mL), 4N HCl (25.9 mL, 103.69 mmol) in dioxane was added. The mixture was stirred at room temperature overnight. The mixture was then concentrated and tritrated with ether. The white solid was filtered and washed with ether, then dried under HV to afford 7-azabicyclo[2.2.1]heptan-1-yl(phenyl)methanone hydrochloride (1.7 g, 86%). LCMS (MS-3), M+H+=202.2 (t =0.33min).

### Step B. Preparation of (7-methyl-7-azabicyclo[2.2.1]heptan-1-yl)(phenyl)methanone from 7-azabicyclo[2.2.1]heptan-1-yl(phenyl)methanone hydrochloride

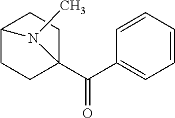

To a reaction vessel containing 5N sodium hydroxide (3.62 mL, 18.09 mmol) was added 7-azabicyclo[2.2.1]heptan-1-yl(phenyl)methanone hydrochloride (2 g, 8.41 mmol) followed by 1,4-dioxane (31.5 mL). The mixture was stirred at room temperature for 30 min, then briefly cooled in an ice bath until the dioxane began to freeze. Dimethyl sulfate (0.881 mL, 9.25 mmol) was added and the reaction mixture was stirred at room temperature for 2 h. The solvent was reduced in volume in vacuo and the residue was partitioned between EtOAc (75 mL) and brine (30 mL). The layers were separated and the aqueous layer was washed with EtOAc. The combined organic layers were dried over MgSO₄, filtered and evaporated in vacuo. The residue was flash chromatographed on silica gel (0-30% EtOAc/Hexane) to give 1.16 g of (7-methyl-7-azabicyclo[2.2.1]heptan-1-yl)(phenyl)methanone as a colorless oil. 1H NMR (500 MHz, chloroform-d) δ ppm 1.42-1.48 (m, 2H), 1.72 (br s, 2H), 1.93-2.00 (m, 2H), 2.13 (s, 3H), 2.13-2.27 (m, 2H), 3.41-3.43 (m, 1H), 7.42-7.45 (m, 2H), 7.52-7.55 (m, 1H), 8.47-8.49 (m, 2H). m/z (ES+), (M+H)+=216.2.

### Step C. Preparation of (S*)-tert-butyl (7-methyl-7-azabicyclo[2.2.1]heptan-1-yl)(phenyl)methylcarbamate and (R*)-tert-butyl (7-methyl-7-azabicyclo[2.2.1]heptan-1-yl)(phenyl)methylcarbamate from (7-methyl-7-azabicyclo[2.2.1]heptan-1-yl)(phenyl)methanone

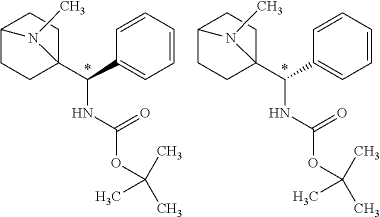

A mixture of 7N Ammonia in MeOH (76 ml, 534.16 mmol), tetraisopropoxytitanium (4.70 ml, 16.02 mmol) and (7-methyl-7-azabicyclo[2.2.1]heptan-1-yl)(phenyl)methanone (1.15 g, 5.34 mmol) was heated to 50° C. overnight in a sealed tube. Then it was cooled to room temperature and sodium borohydride (0.404 g, 10.68 mmol) was added as a solid, and stirred at room temperature for 2 h. 1N NaOH was added (1 mL), followed by celite. The mixture was stirred at room temperature for 1 h then filtered. The solution was concentrated and the crude (7-methyl-7-azabicyclo[2.2.1]heptan-1-yl)(phenyl)methanamine (1.155 g, 5.34 mmol) was dissolved in CH₂Cl₂ (20 mL) and di-tert-butyl dicarbonate (2.480 mL, 10.68 mmol) was added. This reaction mixture was stirred at room temperature overnight, then partitioned between CH₂Cl₂ and water. The layers were separated and the aqueous layer was washed with CH₂Cl₂. The organic extracts were combined, dried over MgSO₄, filtered and concentrated in vacuo. The residue was chromatographed on a basic alumina column to give 1.54 g as a colorless oil. The racemic tert-butyl (7-methyl-7-azabicyclo[2.2.1]heptan-1-yl)(phenyl)methylcarbamate obtained was resolved under supercritical fluid chromatography conditions (liquid CO₂) on a ChiralPak IC column (21.2 mm×150 mm) using 15% methanol containing 0.5% dimethylethylamine at 55 ml/min and a wavelength of 260 nm to afford faster eluting (S*)-tert-butyl(7-methyl-7-azabicyclo[2.2.1]heptan-1-yl)(phenyl)methylcarbamate and slower eluting (R*)-tert-butyl(7-methyl-7-azabicyclo[2.2.1]heptan-1-yl)(phenyl)methylcarbamate. Relative Stereochemistry: In general, the absolute stereochemistry of individual isomers obtained in this manner was not determined. Arbitrary designations were used (R*,S*). (S*)-tert-butyl(7-methyl-7-azabicyclo[2.2.1]heptan-1-yl)(phenyl)methylcarbamate. 1H NMR (300 MHz, chloroform-d) δ ppm 0.93-0.99 (m, 1H), 1.09-1.3 (m, 3H), 1.34 (br. s., 9H), 1.68-1.80 (m, 3H), 1.92-2.05 (m, 1H), 2.24 (s, 3H), 3.23 (t, 1H), 4.64 (br. s., 1H), 5.55 (br. s., 1H), 7.18-7.29 (m, 5H). m/z (ES+), (M+H)+=317.3. (R*)-tert-butyl(7-methyl-7-azabicyclo[2.2.1]heptan-1-yl)(phenyl)methylcarbamate. 1H NMR (300 MHz, chloroform-d) δ ppm 0.93-0.99 (m, 1H), 1.09-1.3 (m, 3H), 1.34 (br. s., 9H), 1.68-1.80 (m, 3H), 1.92-2.05 (m, 1H), 2.24 (s, 3H), 3.23 (t, 1H), 4.64 (br. s., 1H), 5.55 (br. s., 1H), 7.18-7.29 (m, 5H). m/z (ES+), (M+H)+=317.3.

### Step D. Preparation of (R*)-(7-methyl-7-azabicyclo[2.2.1]heptan-1-yl)(phenyl)methanamine bis hydrochloride from (R*)-tert-butyl (7-methyl-7-azabicyclo[2.2.1]heptan-1-yl)(phenyl)methylcarbamate

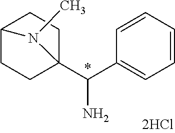

Product was prepared in an analogous manner to the racemate as described in Example 3, Step F to give quantitative yield of (R*)-(7-methyl-7-azabicyclo[2.2.1]heptan-1-yl)(phenyl)methanamine bis hydrochloride

### Step E. Preparation of (R*)-2-fluoro-6-methyl-N-((7-methyl-7-azabicyclo[2.2.1]heptan-1-yl)(phenyl)methyl)benzamide from (R*)-(7-methyl-7-azabicyclo[2.2.1]heptan-1-yl)(phenyl)methanamine bis hydrochloride

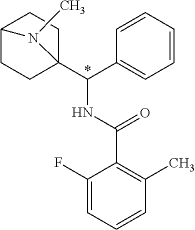

To a reaction vial was added 2-fluoro-6-methylbenzoic acid (19.98 mg, 0.13 mmol), TBTU (41.6 mg, 0.13 mmol), and chiral (7-methyl-7-azabicyclo[2.2.1]heptan-1-yl)(phenyl)methanamine dihydrochloride (25 mg, 0.09 mmol) derived from the second eluting BOC protected isomer. To this was added CH₂Cl₂ (2 mL) and to the resulting suspension was added N,N-diisopropylethylamine (0.098 mL, 0.56 mmol). The reaction mixture (now a solution) was stirred at room temperature overnight. The reaction mixture was partitioned between CH₂Cl₂ and 0.5N NaOH. The layers were separated and the aqueous layer was washed with CH₂Cl₂. The organic extracts were combined and dried over MgSO₄, filtered and concentrated in vacuo. The residue was purified by flash chromatography on silica gel (100% DCM to 5% MeOH in DCM gradient) to give 23 mg of (R*)-2-fluoro-6-methyl-N-((7-methyl-7-azabicyclo[2.2.1]heptan-1-yl)(phenyl)methyl)benzamide. 1H NMR (300 MHz, chloroform-d) δ ppm 0.95-1.06 (m, 1H), 1.11-1.31 (m, 2H), 1.34-1.45 (m, 1H), 1.60-1.88 (m, 3H), 1.95-2.08 (m, 1H), 2.28 (s, 3H), 2.38 (s, 3H), 3.23 (t, J=4.6 Hz, 1H), 5.07 (d, J=4.4 Hz, 1H), 6.87-7.02 (m, 3H), 7.17-7.42 (m, 6H). m/z (ES+), (M+H)+=353.3.

### Example 8

### Preparation of N-((3-bromophenyl)(7-methyl-7-azabicyclo[2.2.1]heptan-1-yl)methyl)-2,6-dimethylbenzamide

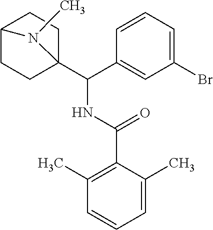

### Step A. Preparation of 7-azabicyclo[2.2.1]heptan-1-yl(3-bromophenyl)methanone hydrochloride from tert-butyl 7-azabicyclo[2.2.1]heptane-7-carboxylate

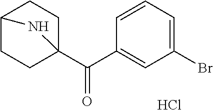

To a stirred solution of tert-butyl 7-azabicyclo[2.2.1]heptane-7-carboxylate (2400 mg, 12.17 mmol) in Et₂O (40 mL), N1,N1,N2,N2-tetramethylethane-1,2-diamine (2.74 mL, 18.25 mmol) was added. The mixture was stirred at room temperature for 5 min, before the dropwise addition of sec BuLi 1.4 M in cyclohexane (10.43 mL, 14.60 mmol) Slight exotherm. The reaction was stirred at room temperature for 15 min, before the addition of 3-bromobenzaldehyde (2251 mg, 12.17 mmol) in 5 mL ether at 0° C. After 30 min at room temperature, the reaction is quenched with aq. NH₄Cl and water. The organic layer was separated. The aqueous layer was extracted with ether. The ether layers were combined and washed with brine, dried over MgSO₄, filtered and concentrated. The residue was purified by silica gel column (40 g, 0%-50% Hex/EA) to afford tert-butyl 1-((3-bromophenyl)(hydroxy)methyl)-7-azabicyclo[2.2.1]heptane-7-carboxylate (2.69 g, 58%). To a stirred cold solution of oxalyl chloride (0.613 ml, 7.02 mmol) in 20 mL DCM at −78° C., DMSO (0.998 ml, 14.05 mmol) in 5 mL DCM was added. The solution was stirred at −78° C. for 10 min, before the addition of alcohol (1.79 g, 4.7 mmol) in 10 mL DCM. Stirred at −78° C. for 20 min before the addition of TEA. Stirred at −78° C. for 10 min before warming to room temperature. The organic was washed with NaHCO₃, dried over MgSO₄, filtered, and concentrated. The crude was purified by silica gel column (0-100% hex/EA, 40 g column) to give tert-butyl 1-(3-bromobenzoyl)-7-azabicyclo[2.2.1]heptane-7-carboxylate (1.9 g, quantitative). To a stirred solution of tert-butyl 1-(3-bromobenzoyl)-7-azabicyclo[2.2.1]heptane-7-carboxylate (1.8 g, 4.73 mmol) in 1,4-dioxane (15 mL), 4N HCl (17.75 mL, 71.00 mmol) in dioxane was added. The mixture was stirred at room temperature overnight. The mixture was then concentrated and tritrated with ether. The white solid was filtered and washed with ether, dried under HV to afford 7-azabicyclo[2.2.1]heptan-1-yl(3-bromophenyl)methanone hydrochloride (1.410 g, 94%) as white solid. 1H NMR (500 MHz, DMSO-d6) δ ppm 1.87 (t, J=9.3 Hz, 2H), 2.08 (t, J=14.6 Hz, 4H), 2.59 (t, J=9.0 Hz, 2H), 4.15 (t, J=4.6 Hz, 1H), 7.57 (t, J=7.9 Hz, 1H), 7.96 (dd, J=7.9, 1.2 Hz, 1H), 8.04-8.11 (m, 2H), 9.62 (br. s., 2H).

### Step B. Preparation of (3-bromophenyl)(7-methyl-7-azabicyclo[2.2.1]heptan-1-yl)methanone from 7-azabicyclo[2.2.1]heptan-1-yl(3-bromophenyl)methanone hydrochloride

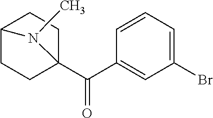

Product was prepared in an analogous manner as described in Example 6, Step B to give (3-bromophenyl)(7-methyl-7-azabicyclo[2.2.1]heptan-1-yl)methanone. 1H NMR (500 MHz, CDCl3) δ ppm 1.40-1.50 (m, 2H), 1.69 (br. s., 2H), 1.96 (t, J=10.8 Hz, 2H), 2.12 (s, 3H), 2.15-2.27 (m, 2H), 3.42 (t, J=4.6 Hz, 1H), 7.32 (t, J=7.9 Hz, 1H), 7.65 (d, J=1.2 Hz, 1H), 8.49 (d, J=7.9 Hz, 1H), 8.61 (s, 1H). m/z (ES+), (M+H)+=294.2; MS-3, HPLC tR=0.58 min.

### Step C. Preparation of (3-bromophenyl)(7-methyl-7-azabicyclo[2.2.1]heptan-1-yl)methanamine from (3-bromophenyl)(7-methyl-7-azabicyclo[2.2.1]heptan-1-yl)methanone

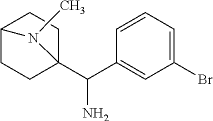

Product was prepared in an analogous manner to the racemate as described in Example 6, Step C to give (3-bromophenyl)(7-methyl-7-azabicyclo[2.2.1]heptan-1-yl)methanamine. The crude racemic amine was then used as it is without resolution. 1H NMR (500 MHz, CDCl3) δ ppm 0.99 (m, 2H), 1.15-1.24 (m, 1H), 1.36 (m, 1H), 1.56-1.73 (m, 4H), 1.94 (m, 1H), 2.09 (m, 1H), 2.26 (s, 3H), 3.23 (t, J=4.7 Hz, 1H), 4.13 (s, 1H), 7.15 (t, J=7.9 Hz, 1H), 7.32 (d, J=7.9 Hz, 1H), 7.36 (d, J=7.9 Hz, 1H), 7.58 (s, 1H). m/z (ES+), (M+H)+=295.1; MS-3, HPLC tR=0.47 min.

### Step D. Preparation of N-((3-bromophenyl)(7-methyl-7-azabicyclo[2.2.1]heptan-1-yl)methyl)-2,6-dimethylbenzamide from (3-bromophenyl)(7-methyl-7-azabicyclo[2.2.1]heptan-1-yl)methanamine

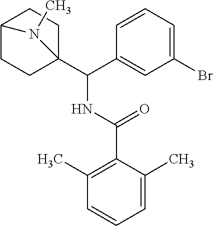

Product was prepared in an analogous manner to the racemate as described in Example 1a, Step G to give racemic N-((3-bromophenyl)(7-methyl-7-azabicyclo[2.2.1]heptan-1-yl)methyl)-2,6-dimethylbenzamide. 1H NMR (500 MHz, CDCl3) δ ppm 1.00-1.12 (m, 1H), 1.19 (m, 2H), 1.39 (m, 1H), 1.57-1.81 (m, 3H), 2.02 (d, J=19.2 Hz, 1H), 2.25 (s, 3H), 2.35 (s, 6H), 3.22 (t, J=4.6 Hz, 1H), 5.03 (br. s., 1H), 6.61 (d, J=1.5 Hz, 1H), 7.03 (d, J=7.6 Hz, 2H), 7.17 (t, J=7.6 Hz, 2H), 7.32 (d, J=7.9 Hz, 1H), 7.39 (d, J=7.9 Hz, 1H), 7.54 (s, 1H). m/z (ES+), (M+H)+=427.2; MS-3, HPLC tR=0.79 min.

### Method 6. Synthesis of N-Alkyl Azabicyclo[2.2.1]heptanes

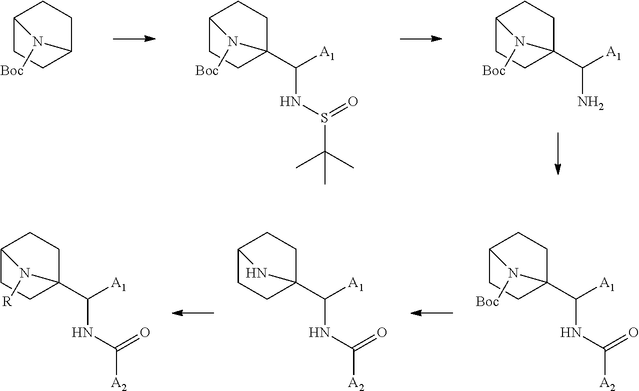

Method 6 depicts a generalized scheme suitable for either stereoselective or racemic synthesis of N-alkyl azabicyclo[2.2.1]heptanes. Those skilled in the art will readily recognize various reagents and intermediates or changes in moieties that could be used to make additional N-alkyl azabicyclo[2.2.1]heptanes. The racemic compounds could either be tested directly or could be readily resolved by Super critical-Fluid Chromatography under suitable conditions.

### Example 9

### Preparation of 2,6-dimethyl-N-((7-methyl-7-azabicyclo[2.2.1]heptan-1-yl)(5-methylfuran-2-yl)methyl)benzamide

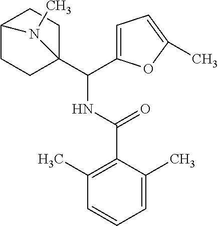

### Step A. Preparation of tert-butyl 1-((1,1-dimethylethylsulfinamido)(5-methylfuran-2-yl)methyl)-7-azabicyclo[2.2.1]heptane-7-carboxylate from tert-butyl 7-azabicyclo[2.2.1]heptane-7-carboxylate

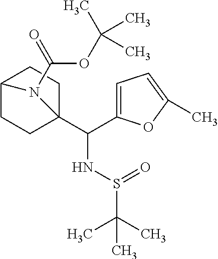

To a round bottom flask was added tert-butyl 7-azabicyclo[2.2.1]heptane-7-carboxylate (2.250 g, 11.41 mmol), Et₂O (15.0 mL), and N1,N1,N2,N2-tetramethylethane-1,2-diamine (2.052 mL, 13.69 mmol). The mixture was stirred at room temp. for ˜5 min. s-BuLi 1.4 M in cyclohexane (9.78 mL, 13.69 mmol) was added dropwise and the reaction was stirred for 10 min. Then a solution of (E)-2-methyl-N-((5-methylfuran-2-yl)methylene)propane-2-sulfinamide (1.6220 g, 7.60 mmol) in Et₂O (6.00 mL) was added dropwise. The reaction was allowed to stir at room temp. for 2.5 hr. The reaction was quenched with saturated NH₄Cl and stirred for ˜15 min. It was then placed into a separatory funnel along with water, saturated NaCl and Et₂O. The organic was collected and aq. extracted 2× more with Et₂O. The combined organics were dried over Na₂SO₄ and rotovaped. The crude material was dissolved in Et₂O and adsorped to silica gel, then purified by silica gel column using hexanes and ether as eluent (1:1 Hex/Ether to 1:4 Hex/Ether) to afford tert-butyl 1-((1,1-dimethylethylsulfinamido)(5-methylfuran-2-yl)methyl)-7-azabicyclo[2.2.1]heptane-7-carboxylate (2.13 g, 37%). m/z (ES+), (M+H)+=411; MS7, HPLC tR=6.80 min.

### Step B. Preparation of tert-butyl 1-(amino(5-methylfuran-2-yl)methyl)-7-azabicyclo[2.2.1]heptane-7-carboxylate from tert tert-butyl 1-((1,1-dimethylethylsulfinamido)(5-methylfuran-2-yl)methyl)-7-azabicyclo[2.2.1]heptane-7-carboxylate

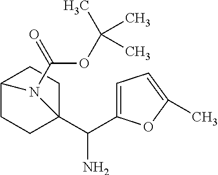

To a round bottom flask was added tert-butyl 1-((1,1-dimethylethylsulfinamido)(5-methylfuran-2-yl)methyl)-7-azabicyclo[2.2.1]heptane-7-carboxylate (2.13 g, 5.18 mmol), dissolved in MeOH (22.0 mL) and cooled to 0° C. Next 4.0 M HCl in dioxane (3.90 mL, 15.55 mmol) was added dropwise. Once complete, the reaction was run at 0° C. for 1.5 hr. To the reaction was added NH4OH until a pH of ˜10 was obtained. It was then placed into a separatory funnel along with H₂O, saturated NaCl and EtOAc. The organic was collected with the aq. being extracted an additional 2× with EtOAC. The combined organics were dried over Na₂SO₄, filtered and concentrated to afford crude tert-butyl 1-(amino(5-methylfuran-2-yl)methyl)-7-azabicyclo[2.2.1]heptane-7-carboxylate (1.38 g, 87%). m/z (ES+), (M+H)+=307; MS7, HPLC tR=2.87 min.

### Step C. Preparation of tert-butyl 1-((2,6-dimethylbenzamido)(5-methylfuran-2-yl)methyl)-7-azabicyclo[2.2.1]heptane-7-carboxylate from tert-butyl 1-(amino(5-methylfuran-2-yl)methyl)-7-azabicyclo[2.2.1]heptane-7-carboxylate

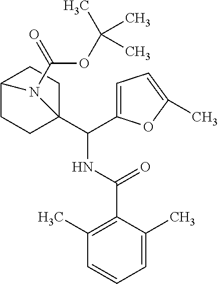

To a round bottom flask was added tert-butyl 1-(amino(5-methylfuran-2-yl)methyl)-7-azabicyclo[2.2.1]heptane-7-carboxylate (1.38 g, 4.51 mmol) dissolved in CH₂Cl₂ (18.0 mL) and cooled to 0° C. Next DIPEA (1.965 mL, 11.28 mmol) was added followed by the dropwise addition of a solution of 2,6-dimethylbenzoyl chloride (0.837 g, 4.96 mmol) in CH₂Cl₂ (2.0 mL). The reaction was stirred at 0° C. for 20 hr. It was added to a separatory funnel along with water, saturated NaCl and CH₂Cl₂. The organic was collected and the aq. extracted 2× more with CH₂Cl₂. The combined organics were dried over Na₂SO₄ and rotovaped. This material was redissolved in Et₂O, adsorped onto silica gel and purified by silica gel column chromatography to afford tert-butyl 1-((2,6-dimethylbenzamido)(5-methylfuran-2-yl)methyl)-7-azabicyclo[2.2.1]heptane-7-carboxylate (1.56 g, 79%). m/z (ES+), (M+H)+=439; MS7, HPLC tR=6.81 min.

### Step D. Preparation of N-(7-azabicyclo[2.2.1]heptan-1-yl(5-methylfuran-2-yl)methyl)-2,6-dimethylbenzamide from tert-butyl 1-((2,6-dimethylbenzamido)(5-methylfuran-2-yl)methyl)-7-azabicyclo[2.2.1]heptane-7-carboxylate

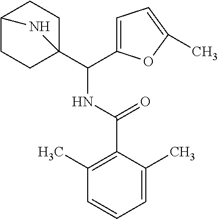

To a round bottom flask was added tert-butyl 1-((2,6-dimethylbenzamido)(5-methylfuran-2-yl)methyl)-7-azabicyclo[2.2.1]heptane-7-carboxylate (1.56 g, 3.57 mmol) dissolved in dioxane (24.0 mL) followed by the dropwise addition of 4.0 M HCl in dioxane (22.0 mL, 85.63 mmol) at room temp. After 1 hr. an additional 24 eq. of 4.0 M HCl in dioxane (22.0 mL, 85.63 mmol) was added and the reaction stirred for 1 hr. CHCl₃ was added to the reaction, it was cooled and then neutralized with saturated NaHCO₃ until a pH of 7-8 was obtained. It was then added to a separatory funnel along with water, saturated NaCl and CHCl₃. The organic was collected and the aq. extracted 2× more with CHCl₃. The combined organics were dried over Na₂SO₄ and rotovaped. This material was adsorped onto silica gel and purified by silica gel column chromatography (MeOH in DCM as eluent) to afford N-(7-azabicyclo[2.2.1]heptan-1-yl(5-methylfuran-2-yl)methyl)-2,6-dimethylbenzamide (0.92 g, 76%). m/z (ES+), (M+H)+=339; MS7, HPLC tR=4.75 min.

### Step E. Preparation of 2,6-dimethyl-N-((7-methyl-7-azabicyclo[2.2.1]heptan-1-yl)(5-methylfuran-2-yl)methyl)benzamide from N-(7-azabicyclo[2.2.1]heptan-1-yl(5-methylfuran-2-yl)methyl)-2,6-dimethylbenzamide

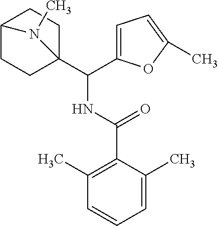

To a round bottom flask was added N-(7-azabicyclo[2.2.1]heptan-1-yl(5-methylfuran-2-yl)methyl)-2,6-dimethylbenzamide (0.92 g, 2.72 mmol) and dissolved in dioxane (14.0 mL). 5 N—NaOH (1.18 mL, 5.85 mmol) was added dropwise and after stirring for 20 min. at room temp., the reaction was cooled to 10-15° C. Next Dimethylsulfate (0.285 mL, 3.00 mmol) was added and the reaction was stirred for 2 hr at 10-15° C. The reaction was added to a reparatory funnel along with H₂O, saturated NaCl and CHCl₃. The organic was collected and the aq. extracted 2× more with CHCl₃. The combined organics were dried over Na₂SO₄ and rotovaped. This material was redissolved in CHCl₃, and adsorped onto silica gel, then purified by silica gel column chromatography (MeOH in DCM as eluent) to afford 2,6-dimethyl-N-((7-methyl-7-azabicyclo[2.2.1]heptan-1-yl)(5-methylfuran-2-yl)methyl)benzamide (0.5 g, 52%). 1H NMR (300 MHz, DMSO) 8.59 (d, 1H), 7.16 (t, 1H), 7.0 (d, 2H), 6.22 (d, 1H), 5.98 (d, 1H), 5.41 (d, 1H), 3.18-3.10 (m, 1H), 2.20 (s, 3H), 2.18 (s, 9H), 1.96-1.56 (m, 4H), 1.37-1.17 (m, 4H). m/z (ES+), (M+H)+=353; MS7, HPLC tR=4.87 min.

Exemplary compounds of Formula I that can be made by the processes described herein include those shown in Table 1:

TABLE 1

| | | | | | Mass spectroscopy |
|---|---|---|---|---|---|
| | | | | | mass ion(s), |
| | | | Synthesis | | (HPLC retention |
| Ex | Structure | IC₅₀ (μM) | Method | Name | time, method) |
| 1 | 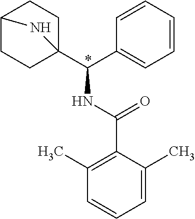 | 0.0998 | 1 | (R*)-N-(7- azabicyclo[2.2.1]heptan-l- yl(phenyl)methyl)-2,6- dimethylbenzamide | 335.2 (0.48 min; MS-1) |
| | (The absolute conformationof this isomer has | | | | |
| | not been determined. Thus, it is unknown | | | | |
| | whether it has the R or S conformation.) | | | | |
| 2 | 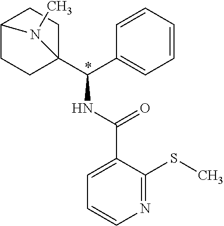 | 0.168 | 2 | (R*)-N-((7-methyl-7- azabicyclo[2.2.1]heptan-1- yl)(phenyl)methyl)-2- (methylthio)nicotinamide | 368.2 (0.46 min; MS-1) |
| | (The absolute conformation of this isomer has | | | | |
| | not been determined. Thus, it is unknown | | | | |
| | whether it has the R or S conformation.) | | | | |
| 3 | 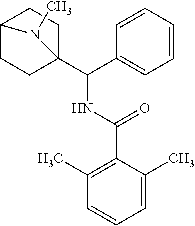 | 0.000772 | 3 | 2,6-dimethyl-N-((7-methyl-7- azabicyclo[2.2.1]heptan-1- yl)(phenyl)methyl)benzamide | 349.3 (0.51 min; MS-1) |
| 4 | 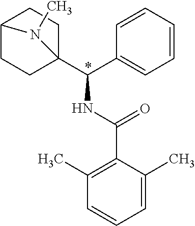 | 0.00282 | 4 | (R*)-2,6-dimethyl-N-((7-methyl- 7-azabicyclo[2.2.1]heptan-1- yl)(phenyl)methyl)benzamide citric acid salt | 349.3 (0.51 min; MS-1) |
| | Isomer 1 | | | | |
| | (This is the chiral isomer of Example 5. The | | | | |
| | absolute conformation of this isomer has not | | | | |
| | been determined. Thus, it is unknown | | | | |
| | whether it has the R or S conformation.) | | | | |
| 5 | 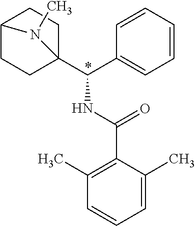 | 0.262 | 4 | (S*)-2,6-dimethyl-N-((7-methyl- 7-azabicyclo[2.2.1]heptan-1- yl)(phenyl)methyl)benzamide citric acid salt | 349.3 (0.51 min; MS-1) |
| | Isomer 2 | | | | |
| | (This is the chiral isomer of Example 4. The | | | | |
| | absolute conformation of this isomer has not | | | | |
| | been determined. Thus, it is unknown | | | | |
| | whether it has the R or S conformation.) | | | | |
| 6 | 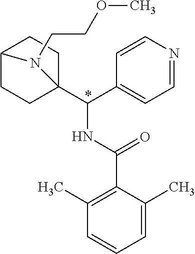 | 0.026 | 5 | (R*)- N-((7-(2-methoxyethyl)-7- azabicyclo[2.2.1]heptan-1- yl)(pyridin-4-yl)methyl)-2,6- dimethylbenzamide | 394.4 (MS-3, 0.52) |
| | Isomer 1 | | | | |
| | (This is the chiral isomer of Example 173. | | | | |
| | The absolute conformation of this isomer has | | | | |
| | not been determined. Thus, it is unknown | | | | |
| | whether it has the R or S conformation.) | | | | |
| 7 | 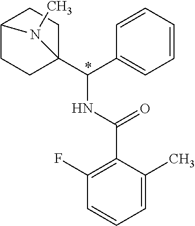 | 0.062 | 5 | 2-fluoro-6-methyl-N-((7-methyl- 7-azabicyclo[2.2.1]heptan-1- yl)(phenyl)methyl)benzamide | 353.3 (MS-3, 0.69) |
| | Isomer 1 | | | | |
| | (This is the chiral isomer of Example 145. | | | | |
| | The absolute conformation of this isomer has | | | | |
| | not been determined. Thus, it is unknown | | | | |
| | whether it has the R or S conformation.) | | | | |
| 8 | 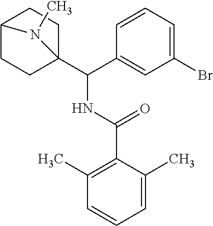 | 0.003 | 5 | N-((3-bromophenyl)(7-methyl-7- azabicyclo[2.2.1]heptan-l- yl)methyl)-2,6- dimethylbenzamide | 427.2, 429.2 (MS-3, 0.79) |
| 9 | 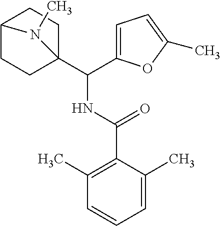 | 0.098 | 6 | 2,6-dimethyl-N-((7-methyl-7- azabicyclo[2.2.1]heptan-1-yl)(5- methylfuran-2- yl)methyl)benzamide | 353.0 (MS-4, 4.87) |
| 10 | 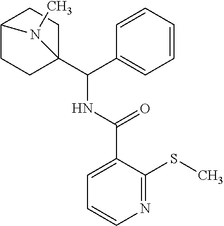 | 0.145 | 3 | N-((7-methyl-7- azabicyclo[2.2.1]heptan-1- yl)(phenyl)methyl)-2- (methylthio)nicotinamide | 368.2 (0.47 min; MS-1) |
| 11 | 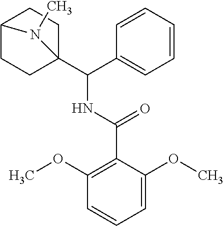 | 0.349 | 3 | 2,6-dimethoxy-N-((7-methyl-7- azabicyclo[2.2.1]heptan-1- yl)(phenyl)methyl)benzamide | 381.3 (0.47 min; MS-1) |
| 12 | 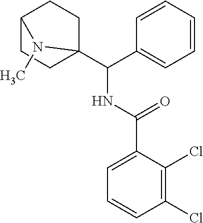 | 0.0336 | 3 | 2,3-dichloro-N-((7-methyl-7- azabicyclo[2.2.1]heptan-1- yl)(phenyl)methyl)benzamide | 389.2, 391.2 (0.56 min; MS-1) |
| 13 | 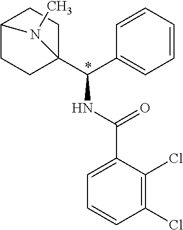 | 0.072 | 2 | (R*)-2,3-dichloro-N-((7-methyl-7- azabicyclo[2.2.1]heptan-1- yl)(phenyl)methyl)benzamide | 389.3, 391.3 (0.54, MS-1) |
| | (The absolute conformation of this isomer has | | | | |
| | not been determined. Thus, it is unknown | | | | |
| | whether it has the R or S conformation.) | | | | |
| 14 | 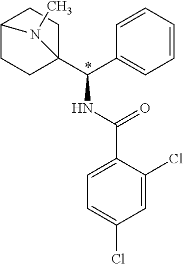 | 0.305 | 2 | (R*)-2,4-dichloro-N((7-methy1-7- azabicyclo[2.2.1]heptan-1- yl)(phenyl)methyl)benzamide | 389.3, 391.3 (0.56 min; MS-1) |
| | (The absolute conformation of this isomer has | | | | |
| | not been determined. Thus, it is unknown | | | | |
| | whether it has the R or S conformation.) | | | | |
| 15 | 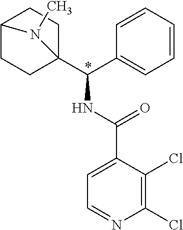 | 0.210 | 2 | (R*)-2,3-dichloro-N-((7-methyl-7- azabicyclo[2.2.1]heptan-1- yl)(phenyl)methyl)isonicotinamide | 390.3, 392.3 (0.51 min; MS-1) |
| | (The absolute conformation of this isomer has | | | | |
| | not been determined. Thus, it is unknown | | | | |
| | whether it has the R or S conformation.) | | | | |
| 16 | 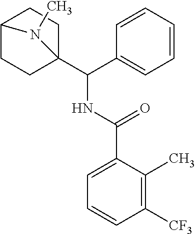 | 0.0186 | 3 | 2-methyl-N-((7-methyl-7- azabicyclo[2.2.1]heptan-1- yl)(phenyl)methyl)-3- (trifluoromethyl)benzamide | 406.3 (0.60 min; MS-1) |
| 17 | 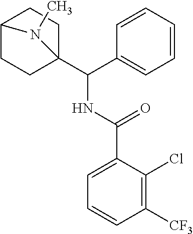 | 0.0179 | 3 | 2-chloro-N-((7-methyl-7- azabicyclo[2.2.1]heptan-1- yl)(phenyl)methyl)-3- (trifluoromethyl)benzamide | 423.2, 425.2 (0.65 min; MS-1) |

Additional compounds made in accordance with the above-described method include those shown below in Tables 2-4. The compounds in Table 2 exhibited an IC₅₀ of less than 0.350 μM. The compounds in Table 3 exhibited an IC₅₀ of from 0.350 μM to 13 μM. And the compounds in Table 4 exhibited an IC₅₀ of greater than 13 μM (i.e., the compounds in Table 4 have relatively less or no activity for the tested target).

TABLE 2
Additional Compounds Exhibiting an IC₅₀ of Less Than 0.350 μM

| | | | Mass Spectroscopy |
|---|---|---|---|
| | | | mass ion(s) |
| | | | HPLC retention time, |
| Example | Structure | IC₅₀ | method |
| 18 | 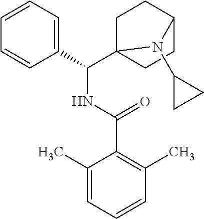 | 0.002 | |
| 19 | 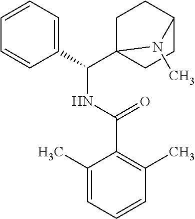 | 0.002 | 352.3 (MS-3, 0.64) |
| 20 | 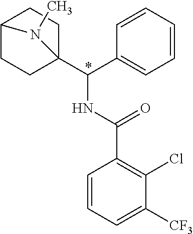 | 0.026 | 424.1 (MS-3, 0.73) |
| | (The absolute conformation of this isomer has not been | | |
| | determined. Thus, it is unknown whether it has the R or | | |
| | S conformation.) | | |
| 21 | 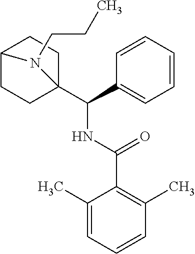 | 0.002 | 374.4 (MS-1, 0.55) |
| 22 | 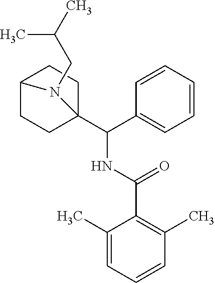 | 0.013 | 391.4 (MS-1, 0.59) |
| 23 | 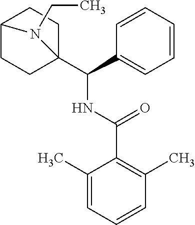 | 0.004 | 363.4 (MS-1, 0.53) |
| 24 | 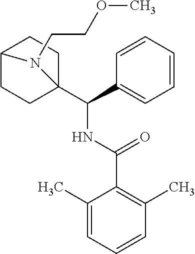 | 0.022 | 393.4 (MS-1, 0.54) |
| 25 | 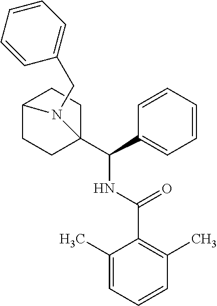 | 0.174 | 425.4 (MS-1, 0.62) |
| 26 | 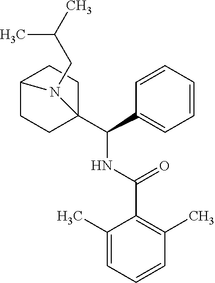 | 0.021 | 391.4 (MS-1, 0.59) |
| 27 | 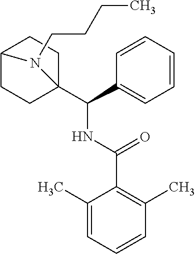 | 0.011 | 391.4 (MS-1, 0.60) |
| 28 | 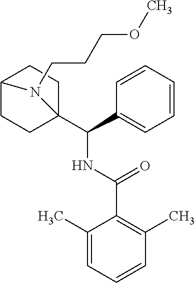 | 0.012 | 407.4 (MS-1, 0.55) |
| 29 | 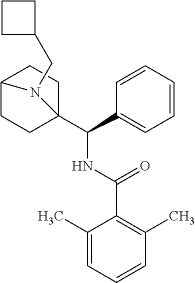 | 0.009 | 403.4 (MS-1, 0.60) |
| 30 | 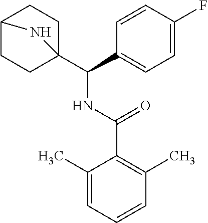 | 0.093 | 353.3 (MS-3, 0.69) |
| 31 | 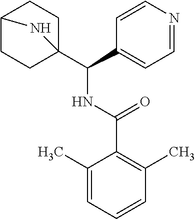 | 0.045 | 336.0 (MS-2, 2.68) |
| 32 | 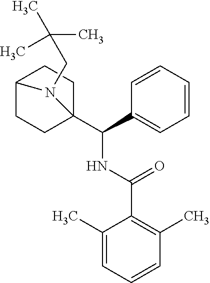 | 0.032 | 407.4 (MS-1, 0.54) |
| 33 | 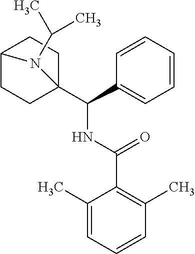 | >0.001 | 377.4 (MS-1, 0.54) |
| 34 | 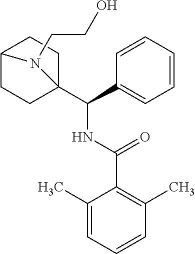 | <0.00128 | 379.4 (MS-3, 0.68) |
| 35 | 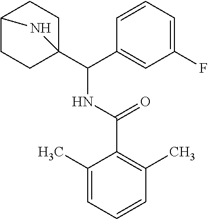 | 0.018 | 353.3 (MS-3, 0.67) |
| 36 | 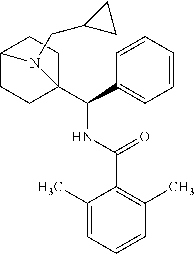 | >0.00113 | 389.4 (MS-3, 0.72) |
| 37 | 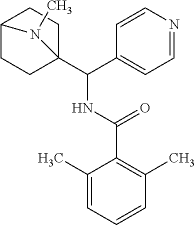 | 0.006 | 350.0 (MS-4, 2.49) |
| 38 | 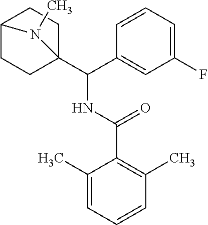 | 0.001 | 367.6 (MS-3, 0.67) |
| 39 | 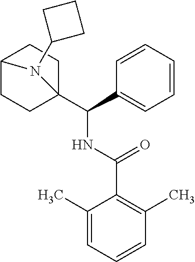 | 0.001 | 389.4 (MS-3, 0.73) |
| 40 | 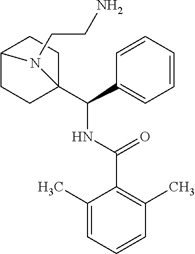 | 0.013 | 378.4 (MS-3, 0.72) |
| 41 | 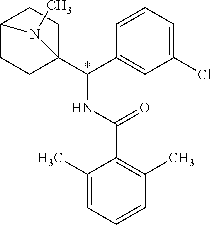 | 0.001 | 383.3 (MS-3, 0.74) |
| | (The absolute conformation of thisisomer has not been | | |
| | determined. Thus, it is unknown whether it has the R or | | |
| | S conformation.) | | |
| 42 | 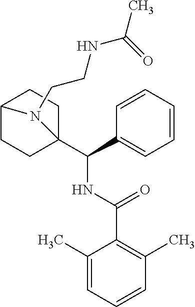 | 0.006 | 420.4 (MS-3, 0.68) |
| 43 | 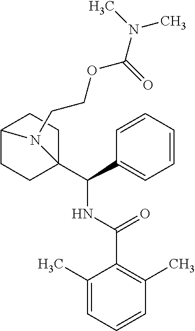 | 0.001 | 450.4 (MS-3, 0.72) |
| 44 | 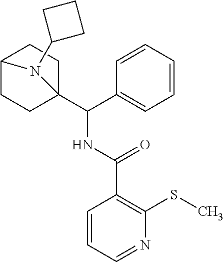 | 0.243 | 408.4 (MS-3, 0.69) |
| 45 | 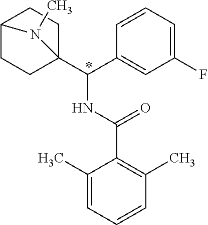 | 0.175 | 367.3 (MS-3, 0.69) |
| | Isomer 1 | | |
| | (This is the chiral isomer of Example 46. The absolute | | |
| | conformation of this isomer has not been determined. | | |
| | Thus, it is unknown whether it has the R or S | | |
| | conformation.) | | |
| 46 | 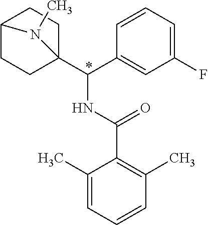 | 0.002 | 367.3 (MS-3, 0.68) |
| | Isomer 2 | | |
| | (This is the chiral isomer of Example 45. The absolute | | |
| | conformation of this isomer has not been determined. | | |
| | Thus, it is unknown whether it has the R or S | | |
| | conformation.) | | |
| 47 | 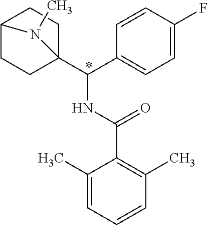 | 0.002 | 367.3 (MS-3, 0.69) |
| | Isomer 1 | | |
| | (This is the chiral isomer of Example 113. The absolute | | |
| | conformation of this isomer has not been determined. | | |
| | Thus, it is unknown whether it has the R or S | | |
| | conformation.) | | |
| 48 | 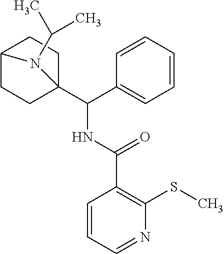 | 0.023 | 396.4 (MS-3, 0.64) |
| 49 | 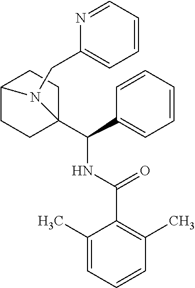 | 0.155 | 426.4 (MS-3, 0.72) |
| 50 | 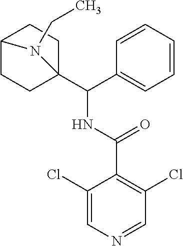 | 0.237 | 404.1 (MS-4, 2.14) |
| 51 | 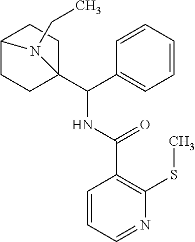 | 0.107 | 382.2 (MS-4, 2.27) |
| 52 | 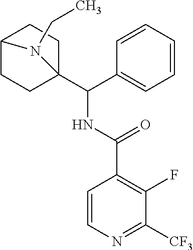 | 0.297 | 422.2 (MS-4, 2.40) |
| 53 | 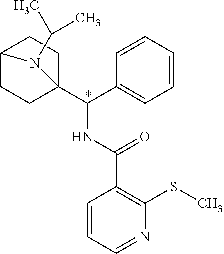 | 0.024 | 396.4 (MS-3, 0.65) |
| | Isomer 1 | | |
| | (This is the chiral isomer of Example 127. The absolute | | |
| | conformation of this isomer has not been determined. | | |
| | Thus, it is unknown whether ithas the R or S | | |
| | conformation.) | | |
| 54 | 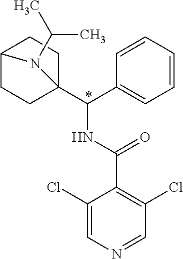 | 0.1 | 418.3, 420.3 (MS-3, 0.63) |
| | Isomer 1 | | |
| | (This is the chiral isomer of Example 128. The absolute | | |
| | conformation of this isomer has not been determined. | | |
| | Thus, it is unknown whether it has the R or S | | |
| | conformation.) | | |
| 55 | 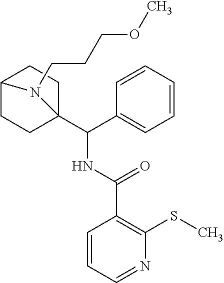 | 0.31 | 426.0 (MS-3, 2.53) |
| 56 | 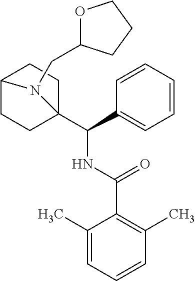 | 0.004 | 419.4 (MS-3, 0.72) |
| 57 | 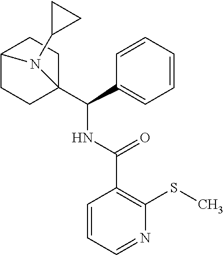 | 0.045 | 394.3 (MS-3, 0.64) |
| 58 | 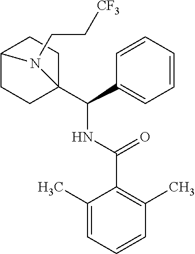 | 0.003 | 431.4 (MS-3, 0.76) |
| 59 | 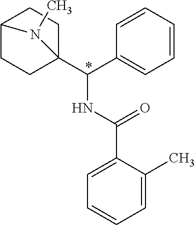 | 0.038 | 335.3 (MS-3, 0.71) |
| | Isomer 1 | | |
| | (This is the chiral isomer of Example 142. The absolute | | |
| | conformation of this isomer has not been determined. | | |
| | Thus, it is unknown whether it has the R or S | | |
| | conformation.) | | |
| 60 | 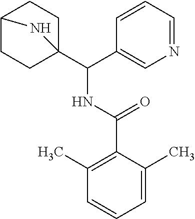 | 0.03 | 336.0 (MS-4, 3.35) |
| 61 | 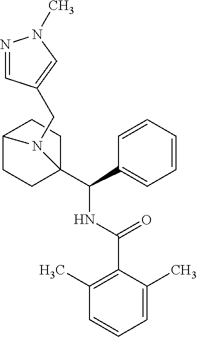 | 0.012 | 429.4 (MS-3, 0.70) |
| 62 | 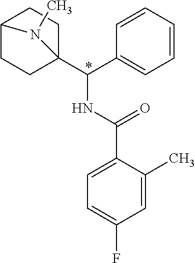 | 0.048 | 353.4 (MS-3, 0.79) |
| | Isomer 1 | | |
| | (This is the chiral isomer of Example 147. The absolute | | |
| | conformation of this isomer has not been determined. | | |
| | Thus, it is unknown whether it has the R or S | | |
| | conformation.) | | |
| 63 | 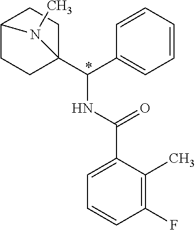 | 0.076 | 353.3 (MS-3, 0.70) |
| | Isomer 1 | | |
| | (This is the chiral isomer of Example 144. The absolute | | |
| | conformation of this isomer has not been determined. | | |
| | Thus, it is unknown whether it has the R or S | | |
| | conformation.) | | |
| 64 | 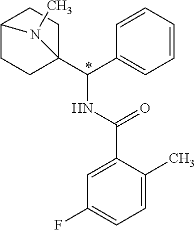 | 0.125 | 353.3 (MS-3, 0.68) |
| | Isomer 1 | | |
| | (This is the chiral isomer of Example 143. The absolute | | |
| | conformation of this isomer has not been determined. | | |
| | Thus, it is unknown whether it has the R or S | | |
| | conformation.) | | |
| 65 | 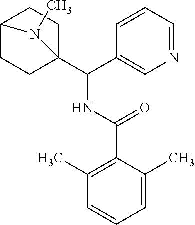 | 0.031 | 350.0 (MS-4, 3.63) |
| 66 | 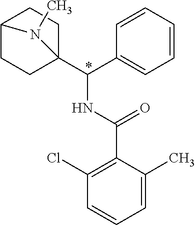 | 0.104 | 370.3 (MS-3, 0.66) |
| | (The absolute conformation of this isomer has not been | | |
| | determined. Thus, it is unknown whether it has the R or | | |
| | S conformation.) | | |
| 67 | 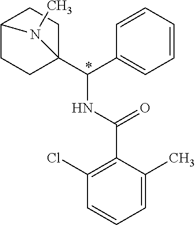 | 0.006 | 369.1 (MS-3, 0.66) |
| | (The absolute conformation of this isomer has not been | | |
| | determined. Thus, it is unknown whether it has the R or | | |
| | S conformation.) | | |
| 68 | 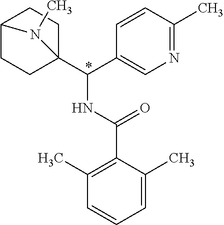 | 0.129 | 364.4 (MS-3, 0.51) |
| 69 | 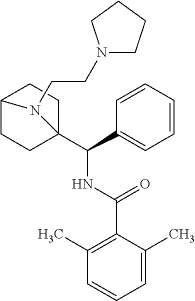 | 0.051 | 432.5 (MS-3, 0.65) |
| 70 | 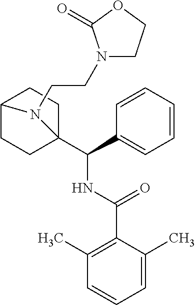 | 0.003 | 448.4 (MS-3, 0.66) |
| 71 | 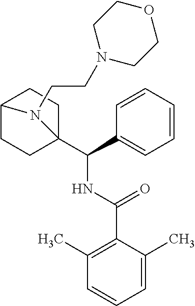 | <0.002 | 448.4 (MS-3, 0.74) |
| 72 | 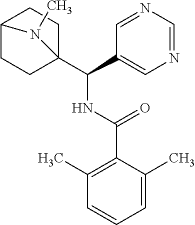 | 0.148 | 351.3 (MS-3, 0.51) |
| 73 | 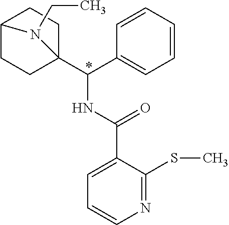 | 0.046 | 382.6 (MS-3, 0.62 ) |
| | Isomer 1 | | |
| | (This is the chiral isomer of Example 148. The absolute | | |
| | conformation of this isomer has not been determined. | | |
| | Thus, it is unknown whether it has the R or S | | |
| | conformation.) | | |
| 74 | 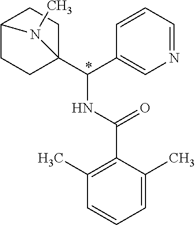 | <0.003 | 350.0 (MS-4, 3.32) |
| | Isomer 1 | | |
| | (This is the chiral isomer of Example 149. The absolute | | |
| | conformation of this isomer has not been determined. | | |
| | Thus, it is unknown whether it has the R or S | | |
| | conformation.) | | |
| 75 | 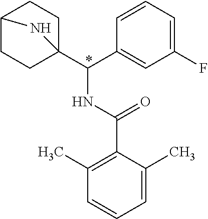 | 0.007 | 353.5 (MS-3, 0.66 ) |
| | Isomer 1 | | |
| | (This is the chiral isomer of Example 76. The absolute | | |
| | conformation of this isomer has not been determined. | | |
| | Thus, it is unknown whether it has the R or S | | |
| | conformation.) | | |
| 76 | 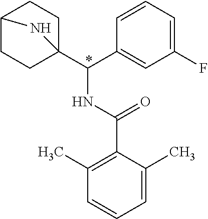 | 0.297 | 353.3 (MS-3, 0.68) |
| | Isomer 2 | | |
| | (This is the chiral isomer of Example 75. The absolute | | |
| | conformation of this isomer has not been determined. | | |
| | Thus, it is unknown whether it has the R or S | | |
| | conformation.) | | |
| 77 | 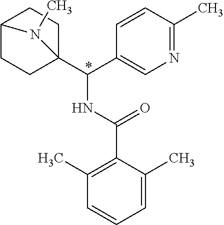 | 0.071 | 364.4 (MS-3, 0.51) |
| | Isomer 1 | | |
| | (This is the chiral isomer of Example 151. The absolute | | |
| | conformation of this isomer has not been determined. | | |
| | Thus, it is unknown whether it has the R or S | | |
| | conformation.) | | |
| 78 | 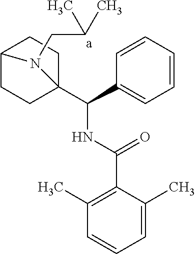 | 0.239 | 392.4 (MS-3, 0.64) |
| | a = Isomer 1/Isomer 2 = 2:1 | | |
| | (The absolute conformation of Isomer 1 and Isomer 2 has | | |
| | not been determined. Thus, it is unknown which isomer | | |
| | is the R isomer and which is the S isomer.) | | |
| 79 | 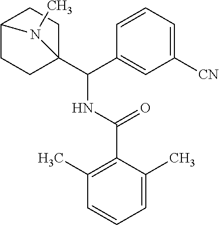 | 0.087 | 374.3 (MS-3, 0.63) |
| 80 | 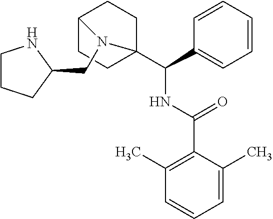 | 0.313 | 418.4 (MS-3, 0.67) |
| 81 | 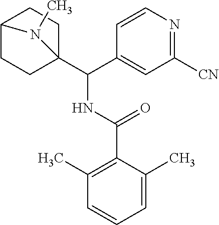 | 0.038 | 364.3 (MS-3, 0.44) |
| 82 | 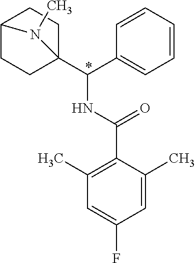 | 0.004 | 367.3 (MS-3, 0.69) |
| | (The absolute conformation of this isomer has not been | | |
| | determined. Thus, it is unknown whether it has the R or | | |
| | S conformation.) | | |
| 83 | 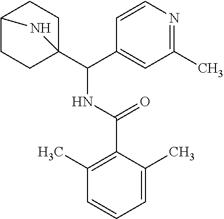 | 0.136 | 350.4 (MS-3, 0.44) |
| 84 | 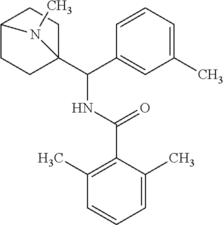 | 0.026 | 363.3 (MS-3, 0.75) |
| 85 | 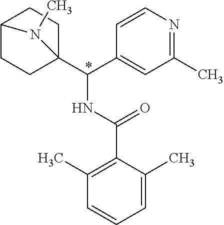 | 0.023 | 364.5 (MS-3, 0.43) |
| | Isomer 1 | | |
| | (This is the chiral isomer of Example 150. The absolute | | |
| | conformation of this isomer has not been determined. | | |
| | Thus, it is unknown whether it has the R or S | | |
| | conformation.) | | |
| 86 | 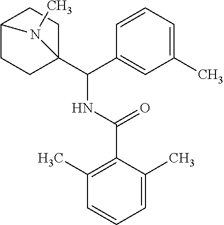 | 0.01 | 364.4 (MS-3, 0.53) |
| 87 | 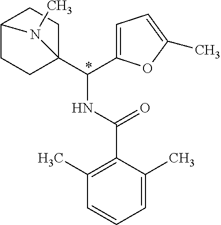 | 0.014 | 353.0 (MS-4, 4.79) |
| | Isomer 1 | | |
| | (This is the chiral isomer of Example 91. The absolute | | |
| | conformation of this isomer has not been determined. | | |
| | Thus, it is unknown whether it has the R or S | | |
| | conformation.) | | |
| 88 | 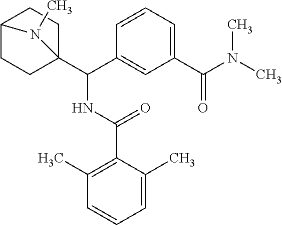 | 0.057 | 420.4 (MS -3,0.60) |
| 89 | 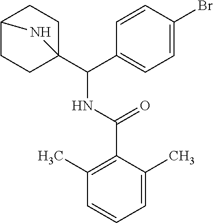 | 0.068 | 413.2 and 415.2 (MS-3, 0.72) |
| 90 | 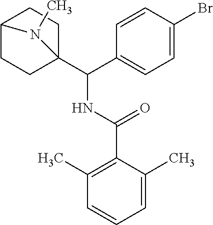 | 0.004 | 427.2 and 429.2 (MS-3, 0.73) |
| 91 | 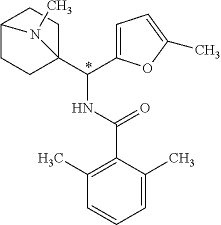 | 0.344 | 353.0 (MS-4, 4.87) |
| | Isomer 2 | | |
| | (This is the chiral isomer of Example 87. The absolute | | |
| | conformation of this isomer has not been determined | | |
| | Thus, it is unknown whether it has the R or S | | |
| | conformation.) | | |
| 92 | 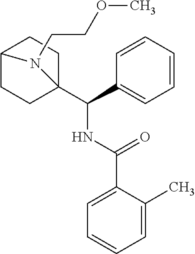 | 0.202 | 379.4 (MS-3, 0.77) |
| 93 | 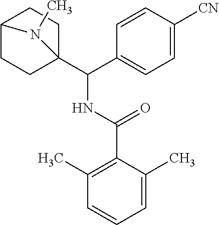 | 0.226 | 374.5 (MS-3, 0.60) |
| 94 | 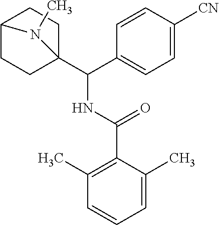 | 0.199 | 363.5 (MS-3, 0.69 ) |
| 95 | 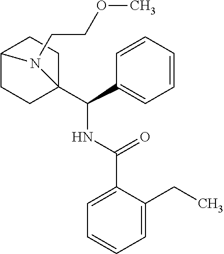 | 0.068 | 393.4 (MS-3, 0.75) |
| 96 | 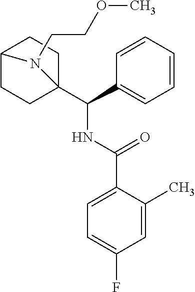 | 0.167 | 397.4 (MS-3, 0.72) |
| 97 | 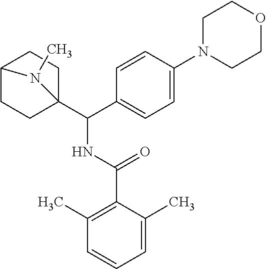 | 0.073 | 434.3 (MS-3, 0.64) |
| 98 | 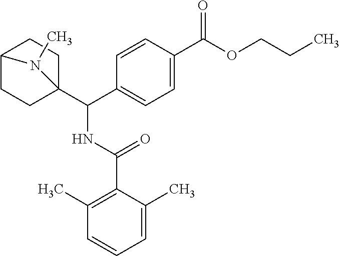 | 0.039 | 435.4 (MS-3, 0.78) |
| 99 | 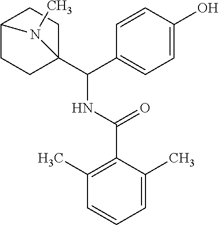 | 0.006 | 365.3 (MS-3, 0.52) |
| 100 | 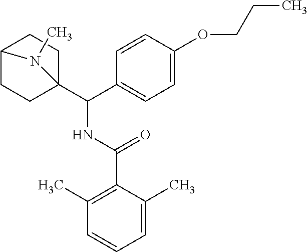 | 0.223 | 407.3 (MS-3, 0.77) |
| 101 | 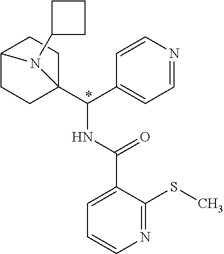 | 0.16 | 409.3 (MS-3, 0.49) |
| | Isomer 1 | | |
| | (This is the chiral isomer of Example 183. The absolute | | |
| | conformation of this isomer has not been determined. | | |
| | Thus, it is unknown whether it has the R or S | | |
| | conformation.) | | |
| 102 | 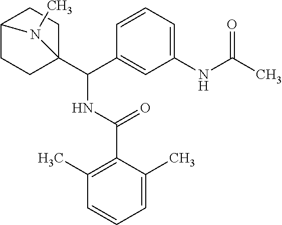 | 0.127 | 406.4 (MS-3, 0.60) |
| 103 | 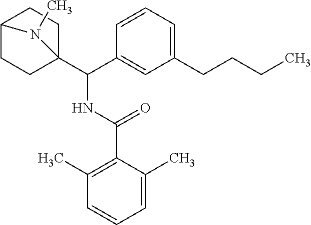 | 0.119 | 405.4 (MS-3, 0.88) |

TABLE 3
Compounds Exhibiting an IC₅₀ of from 0.350 to 13 μM

| | |
|---|---|
| 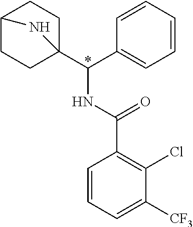 (The absolute conformation of this isomer has not been determined. Thus, it is unknown whether it has the R or S conformation.) | Example 104 |
| 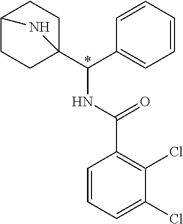 (The absolute conformation of this isomer has not been determined. Thus, it is unknown whether it has the R or S conformation.) | Example 105 |
| 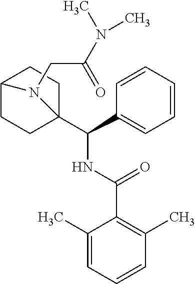 | Example 106 |
| 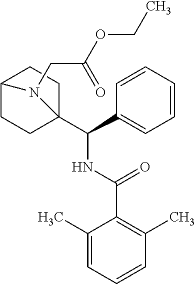 | Example 107 |
| 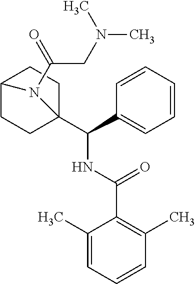 | Example 108 |
| 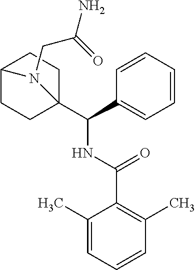 | Example 109 |
| 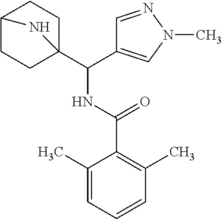 | Example 110 |
| 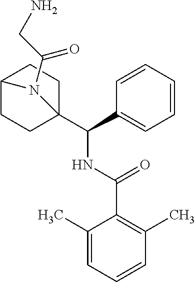 | Example 111 |
| 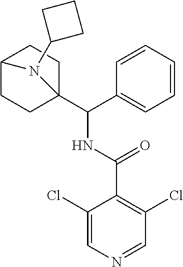 | Example 112 |
| 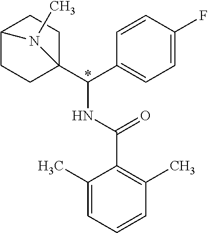 Isomer 2 (This is the chiral isomer of Example 47. The absolute conformation of this isomer has not been determined. Thus, it is unknown whether it has the R or S conformation.) | Example 113 |
| 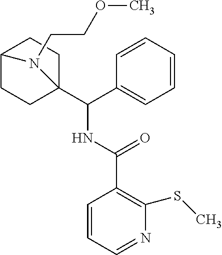 | Example 114 |
| 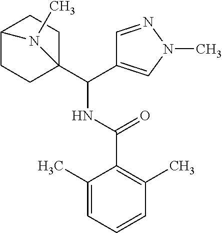 | Example 115 |
| 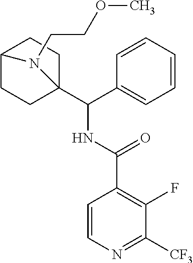 | Example 116 |
| 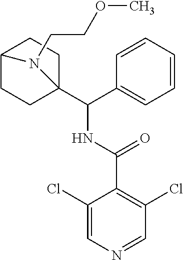 | Example 117 |
| 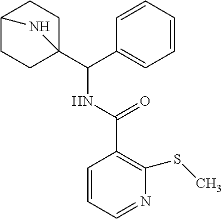 | Example 118 |
| 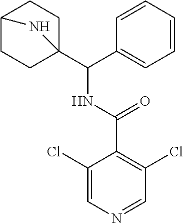 | Example 119 |
| 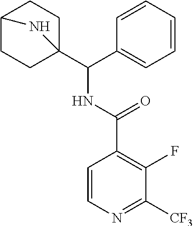 | Example 120 |
| 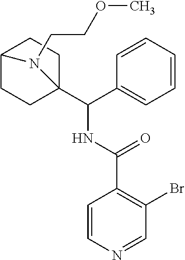 | Example 121 |
| 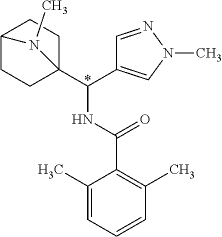 Isomer 1 (This is the chiral isomer of Example 123. The absolute conformation of this isomer has not been determined. Thus, it is unknown whether it has the R or S conformation.) | Example 122 |
| 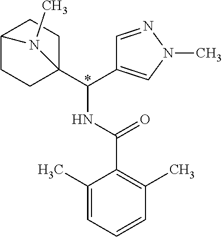 Isomer 2 (This is the chiral isomer of Example 122. The absolute conformation of this isomer has not been determined. Thus, it is unknown whether it has the R or S conformation.) | Example 123 |
| 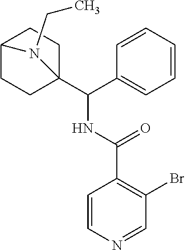 | Example 124 |
| 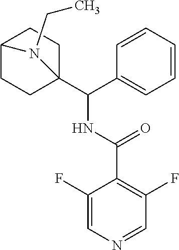 | Example 125 |
| 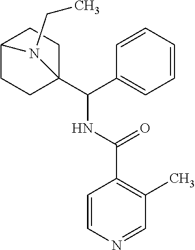 | Example 126 |
| 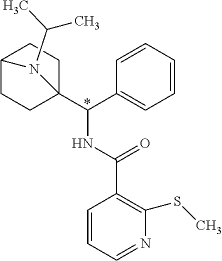 Isomer 2 (This is the chiral isomer of Example 53. The absolute conformation of this isomer has not been determined. Thus, it is unknown whether it has the R or S conformation.) | Example 127 |
| 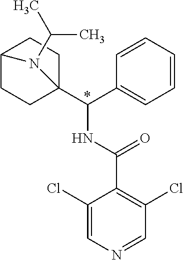 Isomer 2 (This is the chiral isomer of Example 54. The absolute conformation of this isomer has not been determined. Thus, it is unknown whether it has the R or S conformation.) | Example 128 |
| 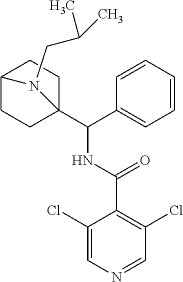 | Example 129 |
| 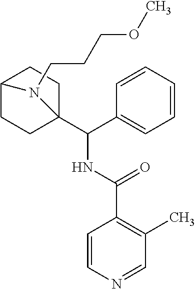 | Example 130 |
| 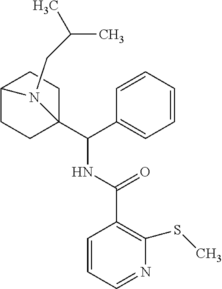 | Example 131 |
| 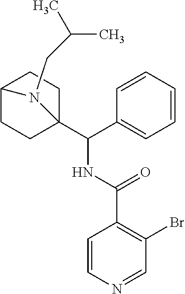 | Example 132 |
| 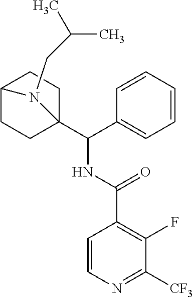 | Example 133 |
| 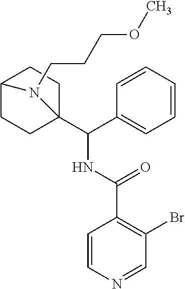 | Example 134 |
| 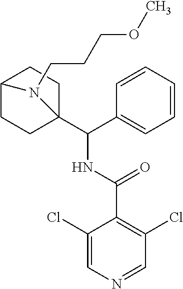 | Example 135 |
| 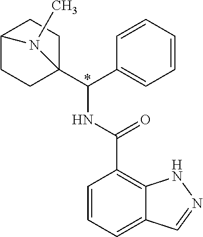 (The absolute conformation of this isomer has not been determined. Thus, it is unknown whether it has the R or S conformation.) | Example 136 |
| 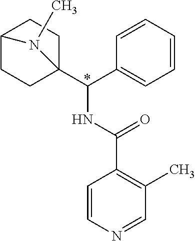 (The absolute conformation of this isomer has not been determined. Thus, it is unknown whether it has the R or S conformation.) | Example 137 |
| 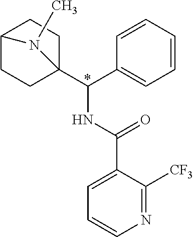 (The absolute conformation of this isomer has not been determined. Thus, it is unknown whether it has the R or S conformation.) | Example 138 |
| 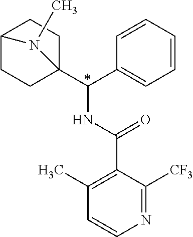 (The absolute conformation of this isomer has not been determined. Thus, it is unknown whether it has the R or S conformation.) | Example 139 |
| 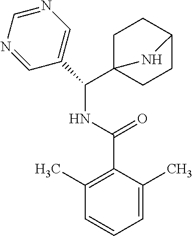 | Example 140 |
| 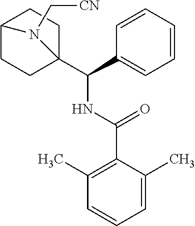 | Example 141 |
| 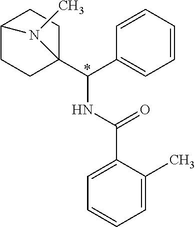 Isomer 2 (This is the chiral isomer of Example 59. The absolute conformation of this isomer has not been determined. Thus, it is unknown whether it has the R or S conformation.) | Example 142 |
| 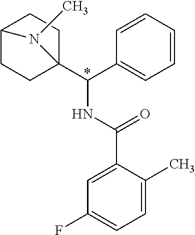 Isomer 2 (This is the chiral isomer of Example 64. The absolute conformation of this isomer has not been determined. Thus, it is unknown whether it has the R or S conformation.) | Example 143 |
| 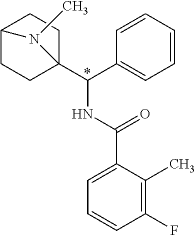 Isomer 2 (This is the chiral isomer of Example 63. The absolute conformation of this isomer has not been determined. Thus, it is unknown whether it has the R or S conformation.) | Example 144 |
| 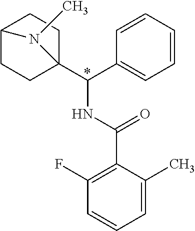 Isomer 2 (This is the chiral isomer of Example 7. The absolute conformation of this isomer has not been determined. Thus, it is unknown whether it has the R or S conformation.) | Example 145 |
| 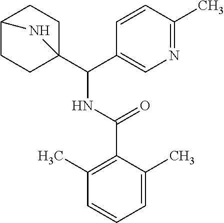 | Example 146 |
| 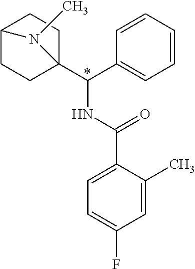 Isomer 2 (This is the chiral isomer of Example 62. The absolute conformation of this isomer has not been determined. Thus, it is unknown whether it has the R or S conformation.) | Example 147 |
| 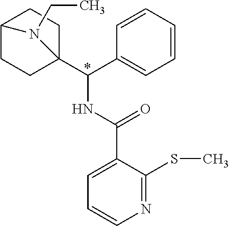 Isomer 2 (This is the chiral isomer of Example 73. The absolute conformation of this isomer has not been determined. Thus, it is unknown whether it has the R or S conformation.) | Example 148 |
| 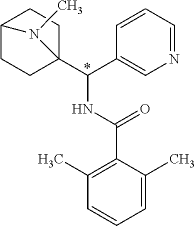 Isomer 2 (This is the chiral isomer of Example 74. The absolute conformation of this isomer has not been determined. Thus, it is unknown whether it has the R or S conformation.) | Example 149 |
| 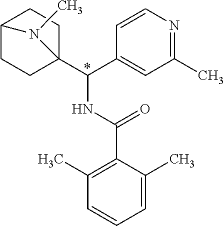 Isomer 2 (This is the chiral isomer of Example 85. The absolute conformation of this isomer has not been determined. Thus, it is unknown whether it has the R or S conformation.) | Example 150 |
| 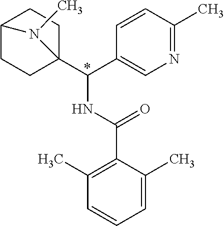 Isomer 2 (This is the chiral isomer of Example 77. The absolute conformation of this isomer has not been determined. Thus, it is unknown whether it has the R or S conformation.) | Example 151 |
| 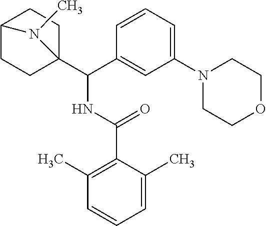 | Example 152 |
| 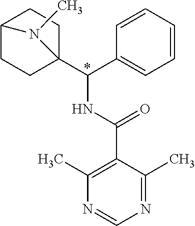 (The absolute conformation of this isomer has not been determined. Thus, it is unknown whether it has the R or S conformation.) | Example 153 |
| 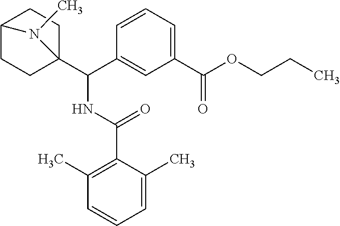 | Example 154 |
| 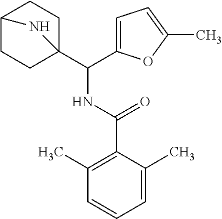 | Example 155 |
| 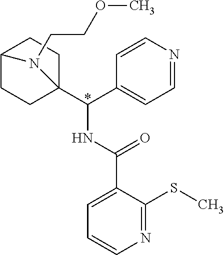 Isomer 1 (This is the chiral isomer of Example 184A. The absolute conformation of this isomer has not been determined. Thus, it is unknown whether it has the R or S conformation.) | Example 156 |
| 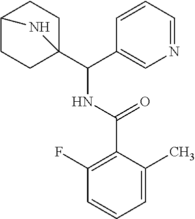 | Example 157 |
| 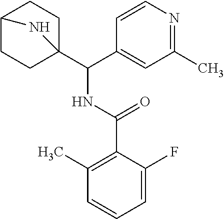 | Example 158 |
| 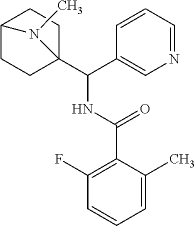 | Example 159 |
| 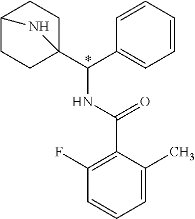 (The absolute conformation of this isomer has not been determined. Thus, it is unknown whether it has the R or S conformation.) | Example 160 |
| 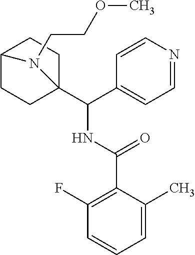 | Example 161 |
| 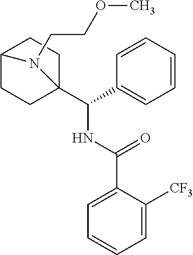 | Example 162 |
| 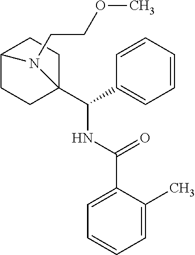 | Example 163 |
| 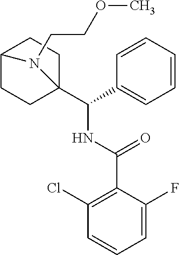 | Example 164 |
| 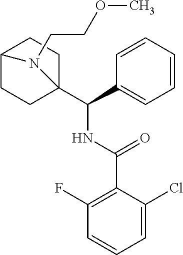 | Example 165 |
| 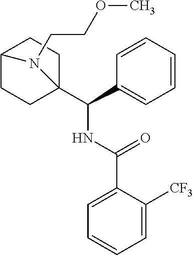 | Example 166 |
| 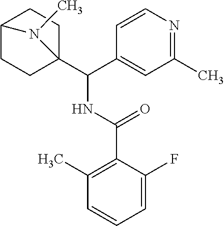 | Example 167 |
| 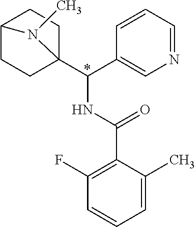 Isomer 1 (This is the chiral isomer of Example 169. The absolute conformation of this isomer has not been determined. Thus, it is unknown whether it has the R or S conformation.) | Example 168 |
| 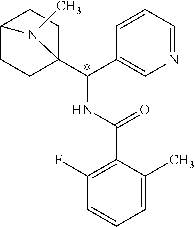 Isomer 2 (This is the chiral isomer of Example 168. The absolute conformation of this isomer has not been determined. Thus, it is unknown whether it has the R or S conformation.) | Example 169 |
| 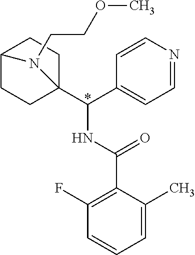 Isomer 1 (This is the chiral isomer of Example 171. The absolute conformation of this isomer has not been determined. Thus, it is unknown whether it has the R or S conformation.) | Example 170 |
| 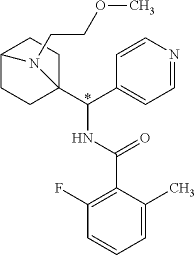 Isomer 2 (This is the chiral isomer of Example 170. The absolute conformation of this isomer has not been determined. Thus, it is unknown whether it has the R or S conformation.) | Example 171 |
| 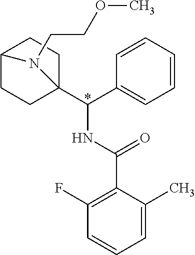 (The absolute conformation of this isomer has not been determined. Thus, it is unknown whether it has the R or S conformation.) | Example 172 |
| 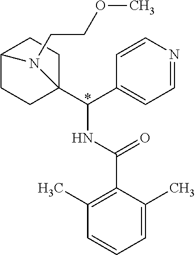 Isomer 2 (This is the chiral isomer of Example 6. The absolute conformation of this isomer has not been determined. Thus, it is unknown whether it has the R or S conformation.) | Example 173 |
| 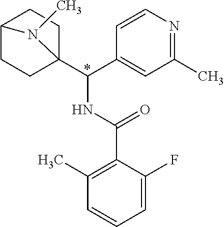 Isomer 1 (This is the chiral isomer of Example 175. The absolute conformation of this isomer has not been determined. Thus, it is unknown whether it has the R or S conformation.) | Example 174 |
| 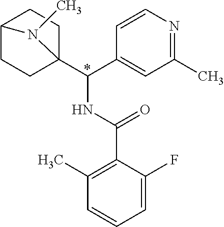 Isomer 2 (This is the chiral isomer of Example 174. The absolute conformation of this isomer has not been determined. Thus, it is unknown whether it has the R or S conformation.) | Example 175 |
| 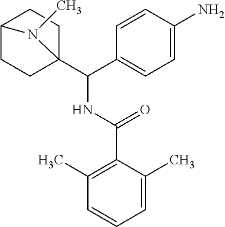 | Example 176 |
| 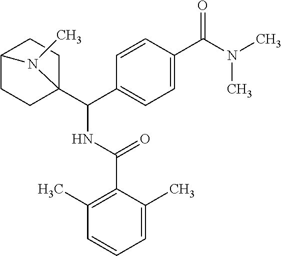 | Example 177 |
| 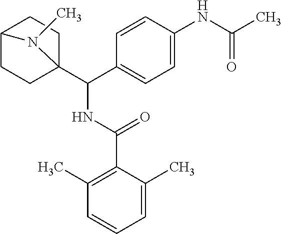 | Example 178 |
| 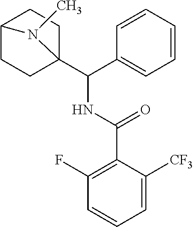 | Example 179 |
| 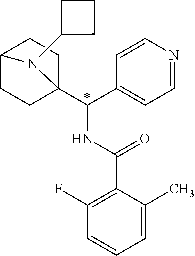 Isomer 1 (This is the chiral isomer of Example 181. The absolute conformation of this isomer has not been determined. Thus, it is unknown whether it has the R or S conformation.) | Example 180 |
| 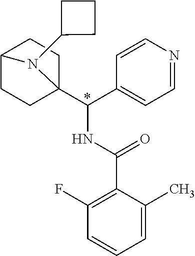 Isomer 2 (This is the chiral isomer of Example 180. The absolute conformation of this isomer has not been determined. Thus, it is unknown whether it has the R or S conformation.) | Example 181 |
| 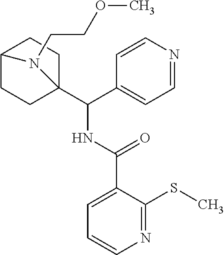 | Example 182 |
| 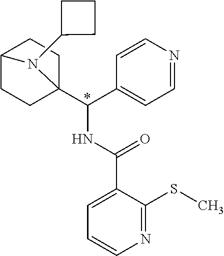 Isomer 2 (This is the chiral isomer of Example 101. The absolute conformation of this isomer has not been determined. Thus, it is unknown whether it has the R or S conformation.) | Example 183 |

TABLE 4
Compounds Exhibiting an IC₅₀ Greater Than 13 μM

| | |
|---|---|
| 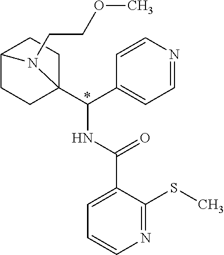 Isomer 2 (This is the chiral isomer of Example 156. The absolute conformation of this isomer has not been determined. Thus, it is unknown whether it has the R or S conformation.) | Example 184A |
| 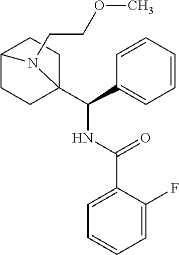 | Example 184B |
| 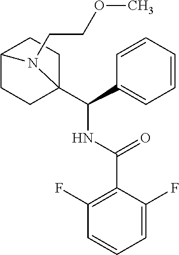 | Example 184C |
| 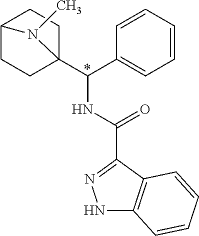 (The absolute conformation of this isomer has not been determined. Thus, it is unknown whether it has the R or S conformation.) | Example 184D |
| 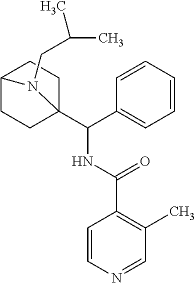 | Example 184E |
| 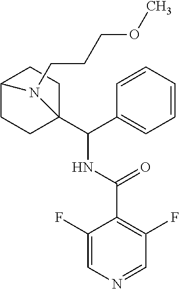 | Example 184F |
| 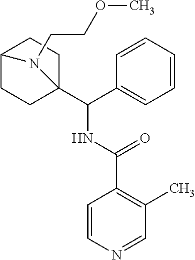 | Example 184G |

Unless otherwise indicated, the following apply in this patent:

The modifier “Cₘ₋Cₙ” means that the modified group contains from m to n carbon atoms. For example, the term “C₁₋C₆-alkyl” means an alkyl group containing from 1 to 6 carbon atoms. Illustrating further, “C₃-C₆-alkenyl” means an alkenyl having from 3 to 6 carbon atoms, with at least one double bond.

The chemical nomenclature used in this patent generally follows the examples and rules stated in Nomenclature of Organic Chemistry, Sections A, B, C, D, E, F, and H, Pergamon Press, Oxford, 1979. Compound names in the above examples were generated using AutoNom 2000 within ISIS/Draw or ChemDraw Ultra 8.0. AutoNom (Automatic Nomenclature) is a chemical-name-generating program that assigns systematic IUPAC (International Union of Pure and Applied Chemistry) chemical names to drawn structures at the press of a button.

The term “hydrocarbon means a chemical structure comprising only carbon and hydrogen atoms.

The term “alkyl” means a fully saturated straight or branched hydrocarbon group. In some embodiments, the alkyl comprises from 1 to 12 carbon atoms. In some embodiments, the alkyl comprises from 1 to 6 carbon atoms. And in some embodiments, the alkyl comprises from 1 to 3 carbon atoms. Examples of alkyl groups include, for example, methyl; ethyl; propyl; isopropyl; 1-methylpropyl; 2-methylpropyl; n-butyl, t-butyl; isobutyl; 3-methylbutyl; pentyl; hexyl; isohexyl; heptyl; 4,4-dimethylpentyl; diethylpentyl; octyl; 2,2,4-trimethylpentyl; nonyl; decyl; undecyl; and dodecyl. An alkyl may be optionally substituted.

The term “alkenyl” is a straight or branched hydrocarbon comprising from 1 to 3 carbon-carbon double bonds. In some embodiments, the chain comprises up to 20 carbon atoms. In some embodiments, the chain comprises up to 10 carbon atoms. In still other embodiments, the chain comprises from 3 to 8 carbon atoms. In still other embodiments, the chain comprises from 3 to 6 carbon atoms. An alkenyl may be optionally substituted.

“Alkynyl” as used herein refers to a straight or branched hydrocarbon comprising from 1 to 3 carbon-carbon triple bonds. In some embodiments, the hydrocarbon comprises up to 20 carbon atoms. In some embodiments, the hydrocarbon comprises up to 10 carbon atoms. In still other embodiments, the hydrocarbon comprises from 2 to 8 carbon atoms. In still other embodiments, the hydrocarbon comprises from 2 to 6 carbon atoms.

The term “alkoxy” means —O-alkyl. Examples of alkoxys include methoxy, ethoxy, propoxy, and butoxy. An alkoxy may be optionally substituted.

The term “cycloalkyl” means a fully saturated cyclic hydrocarbon group. The cycloalkyl may comprise one or more rings. In some embodiments, the cycloalkyl comprises a single ring. In some embodiments, the cycloalkyl comprises from 3 to 10 carbons. In other embodiments, the cycloalkyl comprises from 3 to 6 carbons. Examples of cycloalkyls include, for example, cyclopropyl, cyclobutyl, cyclopentyl, cyclohexyl, cycloheptyl, and cyclooctyl. A cycloalkyl may be optionally substituted.

The term “cycloalkylalkyl” means an alkyl group substituted at its terminal carbon with a cycloalkyl. An example of a cycloalkylalkyl is cyclopropylethyl, which corresponds to:

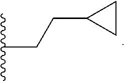

The term “heterocyclyl” means an unsaturated, partially saturated, or fully saturated ring system wherein 1, 2, or 3 of the ring atoms is/are heteroatoms independently selected from N, O, and S, with the remaining ring atoms being carbon. In some embodiments, the heterocyclyl has from 3 to 10 ring atoms. In some embodiments, the heterocyclyl has from 4 to 9 ring atoms. In some embodiments, the heterocyclyl has from 3 to 8 ring atoms. In some embodiments, the heterocyclyl has from 3 to 6 ring atoms. In some embodiments, the heterocyclyl has 5 rings atoms, i.e., it is a 5-membered ring. In some embodiments, the heterocyclyl has 6 rings atoms, i.e.,it is a 6-membered ring. A heterocyclyl may be monocyclic or polycyclic. A heterocyclyl also may be optionally substituted. Examples of single-ring heterocyclyls include furanyl, thienyl (also known as “thiophenyl” and “thiofuranyl”), oxazolyl, isoxazolyl, thiazolyl, isothiazolyl, thiodiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), pyrrolyl, pyrazolyl, imidazolyl, triazolyl, tetrazolyl, oxathiazolyl, oxatriazolyl (including 1,2,3,4-oxatriazolyl and 1, 2,3, 5-oxatriazo IyI), pyridinyl, diazinyl (including pyridazinyl (also known as “1,2-diazinyl”), pyrimidinyl (also known as “1,3-diazinyl”), and pyrazinyl (also known as “1,4-diazinyl”)), triazinyl (including s-triazinyl (also known as “1,3,5-triazinyl”), as-triazinyl (also known 1,2,4-triazinyl), and v-triazinyl (also known as “1,2,3-triazinyl”)), oxathiazinyl (including 1,2,5-oxathiazinyl and 1,2,6-oxathiazinyl), oxepinyl, thiepinyl, dihydrofuranyl, tetrahydrofuranyl, dihydrothienyl (also known as “dihydrothiophenyl”), tetrahydrothienyl (also known as “tetrahydrothiophenyl”), isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl (including 1,2,3-dioxazolyl, 1,2,4-dioxazolyl, 1,3,2-dioxazolyl, and 1,3,4-dioxazolyl), pyranyl (including 1,2-pyranyl and 1,4-pyranyl), dihydropyranyl, tetrahydropyranyl, piperidinyl, piperazinyl, oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxadiazinyl (including 1,4,2-oxadiazinyl and 1,3,5,2-oxadiazinyl), morpholinyl, azepinyl, and diazepinyl. A heterocyclyl alternatively may be 2 or 3 rings fused together, such as, for example, indolizinyl, pyranopyrrolyl, purinyl, imidazopyrazinyl, imidazolopyridazyl, pyridopyridinyl (including pyrido[3, 4-b]-pyridinyl, pyrido[3, 2-b]-pyridinyl, pyrido[4, 3-b]-pyridinyl, and naphthyridinyl), pteridinyl, pyridazinotetrazinyl, pyrazinotetrazinyl, pyrimidinotetrazinyl, pyrindinyl, pyrazolopyrimidinyl, pyrazolopyrazinyl, pyrazolopyridazyl, or 4H-quinolizinyl. In some embodiments, the multi-ring heterocyclyls are selected from indolizinyl, pyranopyrrolyl, purinyl, pyridopyridinyl, pyrindinyl, and 4H-quinolizinyl. Other examples of fused-ring heterocyclyls include benzo-fused heterocyclyls, such as, for example, benzofuranyl (also known as “coumaronyl”), isobenzofuranyl, benzoxazolyl, benzoisoxazolyl (also known as “indoxazinyl”), anthranilyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, benzoisothiazolyl, benzothiadiazolyl, benzoxadiazolyl, indolyl, isoindazolyl (also known as “benzpyrazolyl”), benzoimidazolyl, benzotriazolyl, benzazinyl (including quinolinyl (also known as “1-benzazinyl”) and isoquinolinyl (also known as “2-benzazinyl”)), phthalazinyl, quinoxalinyl, benzodiazinyl (including cinnolinyl (also known as “1,2-benzodiazinyl”) and quinazolinyl (also known as “1,3-benzodiazinyl”)), benzoimidazothiazolyl, carbazolyl, acridinyl, isoindolyl, indoleninyl (also known as “pseudo indolyl”), benzodioxolyl, chromanyl, isochromanyl, thiochromanyl, isothiochromanyl, chromenyl, isochromenyl, thiochromenyl, isothiochromenyl, benzodioxanyl, tetrahydro isoquinolinyl, benzoxazinyl (including 1,3,2-benzoxazinyl, 1,4,2-benzoxazinyl, 2,3,1-benzoxazinyl, and 3,1,4-benzoxazinyl), benzoisoxazinyl (including 1,2-benzisoxazinyl and 1,4-benzisoxazinyl), benzoxadiazinyl, and xanthenyl. In some embodiments, the benzo-fused heterocyclyls are benzofuranyl, isobenzofuranyl, benzoxazolyl, benzoisoxazolyl, anthranilyl, benzothienyl, isobenzothienyl, benzothiazolyl, benzothiadiazolyl, benzoxadiazolyl, indolyl, isoindazolyl, benzoimidazolyl, benzotriazolyl, benzazinyl, phthalazinyl, quinoxalinyl, benzodiazinyl, carbazolyl, acridinyl, isoindolyl, indoleninyl, benzodioxolyl, chromanyl, isochromanyl, thiochromanyl, benzodioxanyl, tetrahydroisoquinolinyl, benzoxazinyl, benzoisoxazinyl, and xanthenyl. The term “2-fused-ring” heterocyclyl means a saturated, non-aromatic partially-saturated, or heteroaryl containing two fused rings. Such heterocyclyls include, for example, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, and benzoisoxazinyl. In some embodiments, the 2-fused-ring heterocyclyls is selected from benzofuranyl, isobenzofuranyl, benzoxazolyl, benzoisoxazolyl, anthranilyl, benzothienyl, isobenzothienyl, benzothiazolyl, benzothiadiazolyl, indolizinyl, pyranopyrrolyl, benzoxadiazolyl, indolyl, isoindazolyl, benzoimidazolyl, benzotriazolyl, purinyl, quinolinyl, isoquinolinyl, pyridopyridinyl, phthalazinyl, quinoxalinyl, benzodiazinyl, pteridinyl, pyrindinyl, isoindolyl, indoleninyl, benzodioxolyl, benzodioxanyl, tetrahydroisoquinolinyl, 4H-quinolizinyl, benzoxazinyl, and benzoisoxazinyl.

The term “heterocycloalkyl” means a fully saturated heterocyclyl. A heterocycloalkyl may be monocyclic or polycyclic. In some embodiments, the heterocycloalkyl has from 3 to 10 ring atoms. In some embodiments, the heterocycloalkyl has from 4 to 9 ring atoms. In some embodiments, the heterocycloalkyl has from 3 to 8 ring atoms. In some embodiments, the heterocycloalkyl has from 3 to 6 ring atoms. In some embodiments, the heterocycloalkyl is a 5-membered ring. In some embodiments, for example, the heterocycloalkyl is a pyrrolidinyl. In other embodiments, the heterocycloalkyl is a tetrahydrofuran. In some embodiments, the heterocycloalkyl is a 6-membered ring. In some embodiments, for example, the heterocycloalkyl is a morpholinyl A heterocycloalkyl may be optionally substituted.

The term “heterocycloalkenyl” means a non-aromatic, partially-saturated saturated heterocyclyl. A heterocycloalkenyl may be monocyclic or polycyclic. In some embodiments, the heterocycloalkenyl has from 4 to 10 ring atoms. In some embodiments, the heterocycloalkenyl has from 4 to 8 ring atoms. In some embodiments, the heterocycloalkenyl is a 5-membered ring. In some embodiments, the heterocycloalkenyl is a 6-membered ring. A heterocycloalkenyl may be optionally substituted.

The term “aryl” means an aromatic hydrocarbon ring structure. The aryl may be monocyclic or polycyclic. Aryls include phenyl and naphthyl. In some embodiments, aryl has 6-10 ring atoms. An aryl may be optionally substituted.

The term “arylalkyl” means an alkyl group substituted at its terminal carbon with an aryl. An example of a arylalkyl is phenylethyl, which corresponds to:

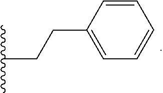

The term “heteroaryl” means an aromatic heterocyclyl. A heteroaryl may be monocyclic or polycyclic. A heteroaryl also may be optionally substituted. In some embodiments, the heteroaryl is a 5-membered ring. In some embodiments, the heteroaryl is a 6-membered ring. In some embodiments, the heteroaryl is an 8-membered bicyclic ring. In some embodiments, the heteroaryl is a 9-membered bicyclic ring. In some embodiments, the heteroaryl is a 10-membered bicyclic ring. Examples of 5-membered heteroaryls include furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, and oxatriazolyl. Examples of 6-membered heteroaryls include pyridinyl, pyrazinyl, pyrimidinyl, pyridazinyl, triazinyl, and oxathiazinyl. Examples of 7-membered heteroaryls include oxepinyl and thiepinyl. Examples of 9-membered heteroaryls include fused-ring systems, such as, for example benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazopyridinyl, and imidazolopyridazyl. Examples of 10-membered heteroaryls include fused-ring systems such as, for example, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl. In some embodiments, the heteroaryl is selected from furanyl, thienyl, oxazolyl, isoxazolyl, thiazolyl, isothiazolyl, oxadiazolyl, pyrazolyl, and imidazolyl. In some such embodiments, the heteroaryl is selected from oxazolyl, isoxazolyl, thiazolyl, imidazolyl, and furanyl. In some embodiments, the heteroaryl is furanyl. In some embodiments, the heteroaryl is pyrazolyl. In some embodiments, the heteroaryl is selected from pyridinyl, pyrazinyl, pyrimidinyl, pyridazinyl, and triazinyl. In some embodiments, the heteroaryl is pyridinyl. In some embodiments, the heteroaryl is pyrimidinyl. In some embodiments, the heteroaryl is selected from benzoxazolyl, benzoisoxazolyl, anthranilyl, benzothienyl, isobenzothienyl, and purinyl. In some embodiments, the heteroaryl is selected from quinolinyl, isoquinolinyl, and benzodiazinyl. In some embodiments, the heteroaryl is imidazopyridinyl, such as, for example:

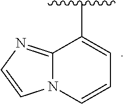

In some embodiments, the heteroaryl is benzoimidazolyl, such as, for example:

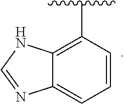

And in some embodiments, the heteroaryl is indazolyl, such as, for example:

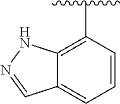

The terms “halogen” and “halo” means chlorine, bromine, fluorine, or iodine. In some embodiments, the halogen atoms in a molecule are selected from the group consisting of chlorine or fluorine. In some embodiments, the halogen atoms in a molecule are chlorine. And in some embodiments, the halogen atoms in a molecule are fluorine. When the term “halo” is used to modify a moiety, that moiety is substituted by one or more independently selected halogens. Thus, for example, “halo-C₁-C₆-alkyl” means a C₁-C₆-alkyl substituted by one or more independently selected halogens. Examples of halo-C₁-C₆-alkyl include —CHCl₂, —CHF₂, and —CF₃.

The term “pharmaceutically acceptable” is used to characterize a moiety (e.g., a salt, dosage form, carrier, or diluent) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “boc” means tert-butoxy carbonyl.

The term “CO₂” means carbon dioxide.

The term “DIPEA” means N,N-diisopropylethylamine.

The term “DMF” means N,N-dimethylformamide.

The term “DMSO” means dimethyl sulfoxide.

The term “DMSO-δ6” means deuterated dimethyl sulfoxide.

The term “EtOAc” means ethyl acetate.

The term “1H NMR” means proton nuclear magnetic resonance.

The term “HOBT” means 1-hydroxybenzotriazole hydrate.

The term “HPLC” means high performance liquid chromatography.

The terms “h” and “hr” means hour or hours.

The term “LCMS” means liquid chromatography mass spectral detection.

The term “m-CPBA” means meta-chloroperbenzoic acid.

The term “m/z” means mass to charge ratio.

The term “MeOH” means methanol.

The term “min” means minute or minutes.

The term “MS” means mass spectrum.

The term “NMR” means nuclear magnetic resonance.

The term “SFC” means supercritical fluid chromatography.

The term “TBTU” means O-(benzotriazol-1-yl)-N,N,N′,N′-tetramethyluronium tetrafluoroborate.

The term “tR” means retention time.

References made in the singular may also include the plural. For example, “a” and “an” may refer to either one or more than one.

The term “optionally substituted” means that the modified group, structure, or molecule may be either: (1) substituted with a substituent at one or more substitutable positions, or (2) not substituted.

The words “comprise,” “comprises,” and “comprising” in this patent (including the claims) are to be interpreted inclusively rather than exclusively. This interpretation is intended to be the same as the interpretation that these words are given under United States patent law.

The above detailed description of illustrative embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A compound or a pharmaceutically acceptable salt thereof, wherein:
the compound corresponds to Formula I: 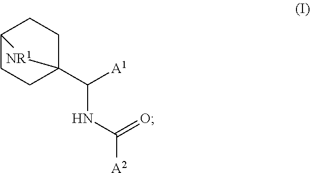
A¹ is selected from:
phenyl optionally substituted with 1, 2, or 3 R⁵ groups; and
a 5- or 6-membered heteroaryl optionally substituted with 1, 2, or 3 R⁷ groups;
A² is selected from:
phenyl substituted with 1, 2, or 3 R² groups; and
a heteroaryl optionally substituted with 1, 2, or 3 R⁶ groups;
each R is independently selected from C₁-C₆-alkyl, C₃-C₈-cycloalkyl-C₁-C₆-alkyl, and NR³R⁴;
R¹ is selected from H, C₁-C₆-alkyl, C₁-C₄-alkoxy-C₁-C₄-alkyl, amino-C₁-C₆-alkyl, cyano-C₁-C₆-alkyl, aminocarbonyl-C₁-C₆-alkyl, hydroxy-C₁-C₆-alkyl, halo-C₃-C₆-alkyl, aminocarbonyloxy-C₁-C₄-alkyl, amino-C₁-C₆-alkylcarbonyl, C₁-C₄-alkylcarbonylamino-C₁-C₄₀-alkyl, C₁-C₄-alkoxycarbonyl-C₁-C₄-alkyl, C₃-C₆ cycloalkyl, 3-6 membered heterocycloalkyl, 5-6 membered heteroaryl, C₃-C₈-cycloalkyl-C₁-C₄-alkyl, aryl-C₁-C₄-alkyl, heterocycloalkyl-C₁-C₄-alkyl, heteroaryl-C₁-C₄-alkyl, and C₃-C₈-alkenyl, wherein:
the C₃-C₈-cycloalkyl-C₁-C₄-alkyl, aryl-C₁-C₄-alkyl, heterocycloalkyl-C₁-C₄-alkyl, and heteroaryl-C₁-C₄-alkyl are optionally substituted with one or more substituents independently selected from halogen and C₁-C₁-alkyl;
the heterocycloalkyl-C₁-C₄-alkyl is optionally substituted with an oxo; and
the amino of the amino-C₁-C₆-alkyl, aminocarbonyl-C₁-C₆-alkyl, aminocarbonyloxy-C₁-C₄-alkyl, and amino-C₁-C₆-alkylcarbonyl is optionally substituted with one or two independently selected C₁-C₄-alkyl;
each R² is independently selected from halogen, —CN, C₂-C₆ alkenyl, C₂-C₆ alkynyl, C₃-C₆ cycloalkyl, heterocyclyl, —SOR, —SO₂R, —NH₂, —SR, C₁-C₆-alkoxy, C₁-C₆-alkyl, —CF₃, and —OCF₃, wherein:
the C₁-C₆-alkyl, C₁-C₆-alkoxy, and C₃-C₆ cycloalkyl is optionally substituted with one or more halogens; and
the heterocyclyl is optionally substituted with 1, 2, or 3 R⁶ groups;
each R⁵ is independently selected from C₁-C₆-alkyl, C₃-C₈-cycloalkyl, C₁-C₆-alkoxy, —CF₃, —OCF₃, —CN, halogen, —SO₂R, —SOR, —SR, C₁-C₄-alkylcarbonylamino, hydroxy, C₁-C₄-alkoxycarbonyl, amino, aminocarbonyl, and heterocyclyl, wherein:
the C₁-C₆-alkyl, C₃-C₈-cycloalkyl, and C₁-C₆-alkoxy is optionally substituted with one or more halogens;
the aminocarbonyl is optionally substituted with up to two independently selected C₁-C₄-alkyl; and
the heterocyclyl is optionally substituted by C₁-C₄-alkyl or halogen;
each R⁶ is independently selected from C₁-C₆-alkyl, C₁-C₆-alkoxy, halogen, —SO₂R, —SOR, —SR, phenyl, —CF₃, —OCF₃, —CN, and heterocyclyl, wherein:
the heterocyclyl is optionally substituted by C₁-C₄-alkyl;
each R⁷ is independently selected from C₁-C₆-alkyl, C₁-C₄-alkoxy, —CF₃, —OCF₃, —CN, —SO₂R, —SOR, —SR, phenyl, heterocyclyl, and C₁-C₄-alkoxy, wherein:
the C₁-C₆-alkyl, C₃-C₈-cycloalkyl, and C₁-C₄-alkoxy is optionally substituted with one or more halogens; and
the heterocyclyl is optionally substituted by C₁-C₄-alkyl or halogen;
each R³ and R⁴ are independently selected from H and C₁-C₆-alkyl; and
any single optical isomer, racemic mixture, or other mixture of optical isomers corresponding to a structure selected from the following (and any salt thereof) are excluded:

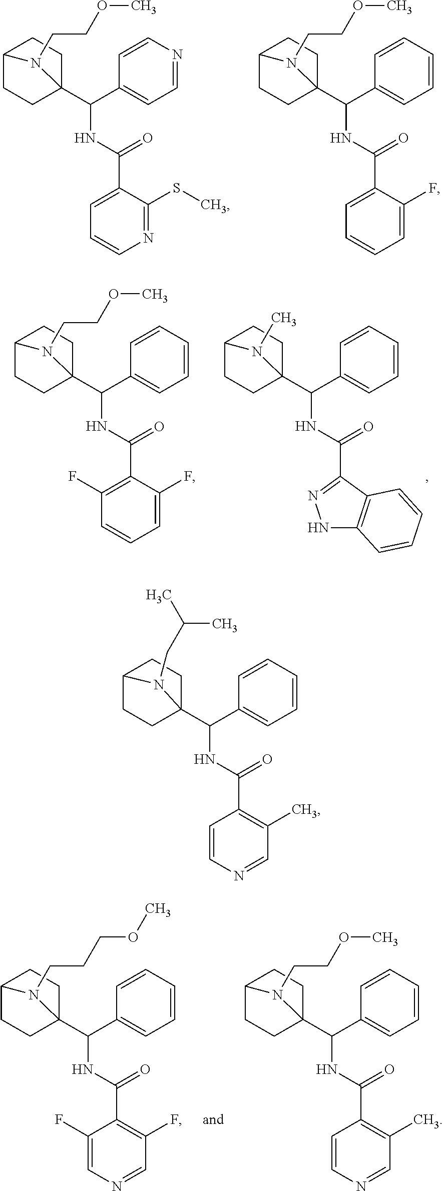

2-5. (canceled)

6. A compound or salt thereof in accordance with claim 1, wherein R¹ is selected from H, C₁-C₆-alkyl, C₃-C₆ cycloalkyl, 3-6 membered heterocycloalkyl, C₃-C₈-cycloalkyl-C₁-C₄-alkyl, aryl-C₁-C₄-alkyl, heterocycloalkyl-C₁-C₄-alkyl, heteroaryl-C₁-C₄-alkyl, and C₃-C₈-alkenyl.

7. A compound or salt thereof in accordance with claim 6, wherein R¹ is hydrogen.

8. A compound or salt thereof in accordance with claim 6, wherein R¹ is C₁-C₆-alkyl.

9. A compound or salt thereof in accordance with claim 8, wherein R¹ is methyl.

10-11. (canceled)

12. A compound or salt thereof in accordance with claim 1, wherein A¹ is phenyl optionally substituted with 1, 2, or 3 R⁵ groups.

13. A compound or salt thereof in accordance with claim 12, wherein A¹ is phenyl.

14. A compound or salt thereof in accordance with claim 1, wherein A² is phenyl substituted with 1, 2, or 3 R² groups.

15. A compound or salt thereof in accordance with claim 14, wherein at least one R² group is C₁-C₆-alkyl.

16. A compound or salt thereof in accordance with claim 15, wherein at least one R² group is methyl.

17. A compound or salt thereof in accordance with claim 1, wherein at least two R² groups are independently selected C₁-C₆-alkyl.

18. A compound or salt thereof in accordance with claim 17, wherein at least two R² groups are methyl.

19. A compound or pharmaceutically acceptable salt thereof in accordance with claim 18, wherein the compound comprises a single optical isomer, racemic mixture, or other mixture of optical isomers corresponding to the following structure:

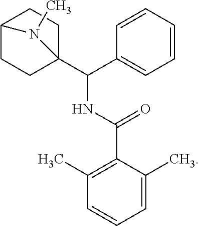

20. A compound or salt thereof in accordance with claim 14, wherein at least one R² group is halogen.

21. A compound or salt thereof in accordance with claim 20, wherein at least one R² group is fluoro.

22. A compound or pharmaceutically acceptable salt thereof in accordance with claim 21, wherein the compound comprises a single optical isomer, racemic mixture, or other mixture of optical isomers corresponding to the following structure:

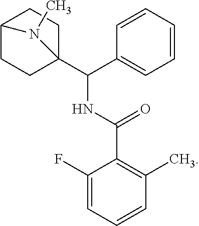

23-27. (canceled)

28. A pharmaceutical composition, wherein the composition comprises:
a compound or a pharmaceutically acceptable salt according to claim 1, and
a pharmaceutically acceptable carrier or diluent.

29-31. (canceled)

32. A method for treating a cognitive disorder or psychosis in a patient in need of such treatment, wherein the method comprises administering a therapeutically effective amount of a compound or salt thereof according to claim 1, the patient.

33-34. (canceled)

35. A method of claim 32, wherein:
the method comprises a method for treating a cognitive disorder, and
the cognitive disorder comprises a disorder selected from schizophrenia, bipolar disorders, mania, manic depression disorders, anxiety disorders, and stress disorders.

36-43. (canceled)

44. A method for treating pain in a patient in need of such treatment, wherein the method comprises administering a therapeutically effective amount of a compound or salt thereof according to claim 1 to the patient.

45-46. (canceled)
